(19) United States
(12) Patent Application Publication
(10) Pub. No.: US 2024/0301345 A1
(43) Pub. Date: Sep. 12, 2024

(54) BIFIDOBACTERIUM LONGUM TRANSITIONAL MICROORGANISMS, COMPOSITIONS AND USES THEREOF

(71) Applicants: The Broad Institute, Inc., Cambridge, MA (US); The General Hospital Corporation, Boston, MA (US); Société des Produits Nestlé S.A., Vevey (CH)

(72) Inventors: Ramnik Xavier, Cambridge, MA (US); Hera Vlamakis, Cambridge, MA (US); Tommi Vatanen, Cambridge, MA (US); Olga Sakwinska, Lausanne (CH); Léa Siegwald, Lausanne (CH); Stéphane Duboux, Lausanne (CH); Catherine Ngom-Bru, Lausanne (CH)

(21) Appl. No.: 18/574,816

(22) PCT Filed: Jun. 28, 2022
(86) PCT No.: PCT/US2022/035310

§ 371 Date: Dec. 28, 2023

Related U.S. Application Data
(60) Provisional application No. 63/216,127, filed on Jun. 29, 2021.

(51) Int. Cl. C12N 1/20; A23L 33/00; A23L 33/135; C12R 1/04

(52) U.S. Cl. CPC C12N 1/205; A23L 33/135; A23L 33/40; C12R 2001/04

(57) ABSTRACT

Described in several exemplary embodiments are Bifidobacterium longum subspecies, microorganisms and formulations thereof. Described in several exemplary embodiments are formulations, such as synthetic formulations, that contain one or more of the Bifidobacterium longum subspecies microorganisms. Described in several embodiments herein is use of the Bifidobacterium longum subspecies microorganisms and formulations thereof, such as in an infant and/or young child.

### CROSS-REFERENCE TO RELATED APPLICATIONS

This application claims the benefit of U.S. Provisional Application No. 63/216,127, filed Jun. 29, 2021. The entire contents of the above-identified applications are hereby fully incorporated herein by reference.

### SEQUENCE LISTING

This application contains a sequence listing filed in electronic form as an ASCII.txt file entitled BROD-5315WP_ST25.txt, created on Jun. 13, 2022, and having a size of 82,361,264 bytes, the content of which is incorporated herein in its entirety.

### TECHNICAL FIELD

The subject matter disclosed herein is generally directed to Bifidobacterium longum transitional clade, microorganisms, formulations thereof, and uses thereof, particularly in infantile populations.

### BACKGROUND

Nutrition plays a critical role in the development across all areas (including cognitive, motor sensory, dentition, musculo-skeletal, immunity, and social development) in infants and young children. Further, the gastrointestinal or “gut” microbiome during infancy can play a significant role in the health and development of the infant both during infancy and later on in life (see e.g., Tanaka and Nakayama. 2017. Allergol. Int. 66(4): 515-522). Various factors, including diet, can significantly influence the microbiome structure and thus influence the health and development of an infant both during infancy and later on in life. During infancy, a mammal, including a human, will transition from a diet that is composed of all or primarily a mother's milk to one of solid foods. This is referred to as the “transitional period”, “transitional feeding period”, or “weaning”. As this occurs, significant changes in the gut microbiome structure can take place due the change in diet and other stressors during that time (see e.g., Vatanen et al., 2019. Nature Microbiology. 4:470-479; Dizzell et al., 2021. PLOS ONE. https://doi.org/10.1371/joumal.pone.0248924; Moore and Townsend. 2019. Open Biol. September; 9(9):190128; Magne et al. 2006. FEMS Microbiology Ecology, 58(3): 563-571; and Edwards C. A. Ann Nutr Metab 2017; 70:246-250). The change in microbiome structure can in turn impact the physiologic, cognitive, anatomical, health or other state or characteristic of the mammal. Although several studies have and are currently investigating the gut microbiome during infancy and young childhood, it and its impact on the immediate and lifelong health and well-being of the infant is far from being well characterized. Paralleling the lack of characterization and understanding of the gut microbiome in infancy and young childhood is also a paucity of compositions and formulations capable of facilitating a healthy gut microbiome appropriate for infant or young child use. As such, there exists a need for improved characterization and understanding of the gut microbiome and compositions and methods to support and/or establish a healthy gut microbiome, particularly in infants and young children.

Citation or identification of any document in this application is not an admission that such a document is available as prior art to the present invention.

### SUMMARY

Described in several example embodiments herein are synthetic formulations comprising one or more Bifidobacterium longum (B. longum) transitional clade microorganism which has at least 98.6% Average Nucleotide Identity (ANI) with at least one strain selected in the group consisting of: CNCM I-5683, CNCM I-5684, CNCM I-5685, CNCM I-5686 and CNCM I-5687.

In certain example embodiments, the synthetic formulation comprises one or more Bifidobacterium longum (B. longum) transitional clade microorganisms characterized by the presence of all genes from Table 1.

Described in several example embodiments herein are synthetic formulations comprising one or more Bifidobacterium longum (B. longum) transitional clade microorganisms characterized by the presence of all genes from Table 2.

In certain example embodiments of the synthetic formulations herein, the one or more B. longum microorganisms is not of the subspecies B. longum subspecies longum or B. longum subspecies. infantis.

In certain example embodiments of the synthetic formulations herein, the one or more B. longum microorganisms are isolated from a human.

In certain example embodiments of the synthetic formulations herein, the synthetic formulations further comprise a fat source, a protein source, a carbohydrate source, a dietary fiber source, or a combination thereof. In certain example embodiments, the fat source is a milk derived fat source or equivalent thereof, wherein the protein source is a milk derived protein source or equivalent thereof, and/or wherein the carbohydrate source is a milk derived carbohydrate source or equivalent thereof. In certain example embodiments, the dietary fiber is a prebiotic fiber. In certain example embodiments, the one or more B. longum microorganisms are associated with the prebiotic fiber.

In certain example embodiments, the synthetic formulation is a milk fortifier or milk replacement.

In certain example embodiments, the synthetic formulation is adapted for infant use.

Described in certain example embodiments herein is a genetically modified microorganism or population thereof, wherein the microorganism is not of a Bifidobacterium longum transitional clade and is genetically modified to have modified expression of one or more genes from Table 1.

In certain example embodiments, the genetically modified microorganism is a Bifidobacterium species that is not of a B. longum transitional clade.

Described in certain example embodiments herein are synthetic formulations comprising the genetically modified microorganism or population thereof described in any of the preceding paragraphs and/or is as described elsewhere herein.

Described in certain example embodiments herein are methods for, in an infant, promoting/assisting transition from a milk-based diet to solid food and/or promoting gut microbiota adapted to metabolize both milk derived carbohydrates and fibers or any derivative thereof comprising administering a synthetic formulation of any one of the preceding paragraphs and/or is as described elsewhere herein to the infant.

Described in certain example embodiments herein are methods for promoting/assisting transition from a milk based diet to solid food and/or promoting gut microbiota adapted to metabolize both milk derived carbohydrates and fibers or a derivative thereof in an infant comprising administering, to the infant, one or more Bifidobacterium longum (B. longum) transitional clade microorganism which has at least 98.6% Average Nucleotide Identify (ANI) with at least one strain selected in the group consisting of: CNCM I-5683, CNCM I-5684, CNCM I-5685, CNCM I-5686 and CNCM I-5687.

In certain example embodiments, the one or more Bifidobacterium longum (B. longum) microorganisms characterized by the presence of all genes from Table 1. In certain example embodiments, the one or more B. longum microorganisms is not of the subspecies B. longum subspecies. longum or B. longum subspecies. infantis. In certain example embodiments, the one or more B. longum microorganisms are isolated from a human.

Described in certain example embodiments herein are methods of identifying a gastrointestinal microbiome modulating agent comprising administering an amount of a test agent to a subject; and determining a microbiome structure of a gastrointestinal tract of the subject by at least determining the relative abundance of one or more one or more Bifidobacterium longum (B. longum) microorganisms characterized by at least 98.6% Average Nucleotide identity (ANI) with at least one strain selected in the group consisting of: CNCM I-5683, CNCM I-5684, CNCM I-5685, CNCM I-5686 and CNCM I-5687, wherein the gastrointestinal microbiome modulating agent is effective to increase or decrease the relative abundance of one or more of the one or more B longum microorganism characterized by the presence of the transitional clade genes.

In certain example embodiments, the one or more B. longum microorganisms is characterized by the presence of all genes from Table 1.

In certain example embodiments, the one or more B. longum microorganisms is not of the subspecies B. longum subspecies. longum or B. longum subspecies infantis.

In certain example embodiments, the one or more B. longum microorganisms are isolated from a human.

In certain example embodiments, the test agent is provided in a formulation.

Described in several example embodiments herein is a Bifidobacterium longum (B. longum) microorganism characterized by at least 98.6% Average Nucleotide identity (ANI) with at least one strain selected in the group consisting of: CNCM I-5683, CNCM I-5684, CNCM I-5685, CNCM I-5686 and CNCM I-5687, and combinations thereof.

Described in several example embodiments herein is a Bifidobacterium longum (B. longum) microorganisms, wherein the B. longum microorganism is characterized by the presence of all genes from Table 1.

Described in several example embodiments herein is a Bifidobacterium longum (B. longum) microorganism as in any one of the preceding paragraphs and/or is as described elsewhere herein, wherein the B. longum microorganisms is not of the subspecies B. longum subspecies longum or B. longum subspecies infantis.

Described in several example embodiments herein is a Bifidobacterium longum (B. longum) microorganism for as in any one of the preceding paragraphs and/or is as described elsewhere herein, which is isolated from human.

Described in several example embodiments herein is a combination of a microorganism as described in any one of the preceding paragraphs and/or as described elsewhere herein, milk derived carbohydrates, and dietary fibers.

Described in several example embodiments herein is a synthetic composition comprising a microorganism as described in any one of the preceding paragraphs and/or is as described elsewhere herein.

Described in several example embodiments herein is a synthetic composition according to any one of the preceding paragraphs and/or is as described elsewhere herein for use in promoting/assisting transition from a milk-based diet to solid food in infants (as of the age of 4 months) and/or in young children and/or promoting in infants (as of the age of 4 months) and/or in young children a gut microbiota adapted to metabolize both milk derived carbohydrates and fibers or any derivative thereof.

Described in several example embodiments herein is the use of a microorganism as described in any one of the preceding paragraphs and/or is as described elsewhere herein for promoting/assisting transition from a milk based diet to solid food in infants (as of the age of 4 months) and/or in young children and/or in promoting in infants (as of the age of 4 months) and/or in young children a gut microbiota adapted to metabolize both milk derived carbohydrates and fibers or any derivative thereof.

Described in several example embodiments herein are methods for promoting/assisting transition from a milk based diet to solid food in infants (as of the age of 4 months) and/or in young children and/or in promoting in infants (as of the age of 4 months) and/or in young children a gut microbiota adapted to metabolize both milk derived carbohydrates and fibers or any derivative thereof by administering a microorganism as described in any one of the preceding paragraphs and/or is as described elsewhere herein to such infants.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A diversity box plot showing an increasing microbial complexity with age.

FIG. 2—Graphs showing a comparison of HMO cluster genes (used to detect B. longum subspecies infantis in DIABIMMUNE and TEDDY) abundance to core genes to determine the relative abundance wrt. B. longum (HUMAnN2 B. longum-stratified CPM) (see also Vatanen et al., Nat Microbiol. 2019 March; 4(3):470-479 and Vatanen et al., Nature. 2018 October; 562(7728):589-594).

FIG. 3—Graphs showing a discrepancy between B. longum relative abundance (MetaPhlAn) and median core genome coverage (StrainPhlAn).

FIGS. 4-5—Graphs showing more polymorphisms in B. longum during the transition from breast feeding to solid foods.

FIGS. 6A-6C and 7A-7D—show the general results of a strain analysis that included seven additional B. longum genomes.

FIG. 8—Shows analysis results that demonstrate a longitudinal B. longum subspecies infantis to Clade 2 (see e.g., FIG. 7C) to Clade 3 transition (see e.g., FIG. 7D) with the introduction of solid foods and weaning.

FIG. 9—A graph showing that B. longum subspecies infantis is present in most samples from Dhaka babies.

FIG. 10—A graph showing that B. longum subspecies infantis is less common in non-breast-fed infants.

FIG. 11—A graph showing that B. longum subspecies infantis is more abundant than other B. longum strains.

FIG. 12—A graph showing that the abundance of B. longum subspecies infantis and other B. longum strains differ during breastfeeding.

FIG. 13—A graph showing that B. longum is ubiquitous with longitudinal strain shifts, including a change in B. longum subspecies infantis.

FIG. 14—Shows a general timeline for sample collection from 222 babies that were followed in Dhaka, Bangladesh. Metagenomic sequencing analysis was performed on 1, 314 stool samples with an average of 26 million reads per sample.

FIG. 15—Graphs showing the number of assembled Bifidobacterium genomes per species as a function of genome completeness. The number of genomes with completeness >90 is shown for each species. Only species with more than 50 genomes are shown.

FIG. 16—Graphs showing the average number of high quality (HQ) genomes per sample and timepoint.

FIG. 17—Graphs showing the number of B. longum subspecies infantis and B. longum subspecies longum genomes (completeness >90) over time.

FIG. 18—Graph showing that there were no multiple HQ genomes (completeness >90) B. longum genomes. N=8 samples with second genome with completeness >50.

FIG. 19—Graph showing the number of genomes vs. number of genes demonstrating how common the gene is among MAGs+reference genomes. 234 HQ MAGs were labeled as B. longum subspecies longum or B. longum subspecies infantis. A pangenome was built together with 25 B. longum subspecies longum/B. longum subspecies infantis reference genomes. 259 “unique” genomes in total. Genomes harbored 484,213 genes in total (sum of genes on all genomes). Genes with >95% identity were considered non-redundant genes. The pangenome harbored 13,834 non-redundant genes.

FIGS. 20A-20E—MAG analysis demonstrating that three clades were clearly separated on reference genomes.

FIG. 21—MAG analysis demonstrating that B. longum MAGs are clearly divided between B. longum subspecies longum and a B. longum transitional clade. MAGs from the B. longum transitional clade look more like B. longum subspecies suis than other reference genomes.

FIG. 22—A phylogeny map and heat map generated using the most prevalent marker genes to estimate and show clade abundance. Points on the phylogeny show the dominant strain by marker gene abundance. The heatmap shows clade abundance within B. longum.

FIG. 23—A ternary/simplex graph showing the succession from B. longum subspecies infantis to B. longum “transitional” clade to B. longum subspecies longum. B. longum subspecies infantis and B. longum “transitional” clade coexist while B. longum subspecies infantis and B. longum subspecies longum do not.

FIG. 24—A workflow of a comprehensive, taxonomy-aware functional annotation of genome assemblies.

FIG. 25—A graph showing distinct longitudinal trends in B. longum subspecies.

FIGS. 26A-26B—MAG analyses demonstrating that the B. longum MAGs are still clearly divided between B. longum subspecies longum and a B. longum transitional clade. The MAGs from the B. longum transitional clade still look more like B. longum subspecies suis than the other reference genomes.

FIG. 27—A comparison of abundance estimates between previous MAG analysis data (see e.g., FIGS. 20A-20E).

FIG. 28—A phylogeny map and heat map generated using the most prevalent marker genes of the expanded dataset to estimate and show clade abundance. Points on the phylogeny show the dominant strain by marker gene abundance. The heatmap shows clade abundance within B. longum.

FIGS. 29A-29C—Graphs showing the relative abundance of B. longum subspecies infantis (FIG. 29A), B. longum transitional clade (FIG. 29B), and B. longum subspecies longum (FIG. 29C).

FIG. 30—A heatmap of selected clade-specific gene functions among isolate genomes (or B. longum subspecies longum references).

FIG. 31—A neighborhood of the beta-lactamase gene on a B. longum transitional clade isolate.

FIG. 32—Nucleotide alignment of B. longum isolates. The overlapping region in the end of the genes (top is B. longum transitional clade beta-lactamase genes, bottom is B. longum subspecies infantis beta-lactamase genes) on nucleotide level (black indicates match). The average nucleotide identity was 52% between the clades.

FIG. 33—Amino acid alignment of B. longum isolates. Overlapping region on grayscale by amino acid seq. Amino acid sequences are distinct per subspecies clades. The average amino acid identity was 34% between the clades.

FIG. 34—A heatmap showing significant rank-correlations between B. longum transitional clade and other species.

FIG. 35—A graph showing the relative abundances of Bifidobacterium longum subspecies overtime.

FIG. 36 shows Average Nucleotide Identity (ANI) UPGMA based phylogenetic tree computed with OrthoANIu software showing separation of the Bifidobacterium subspecies. The scale represents the percentage (%) of identity at each branch point.

FIG. 37—Cell density of selected B. longum subspecies suis, B. longum subspecies. suillum and newly described B. longum clade strains after 48 h growth on glucose or an HMO mix as sole carbon source (0.3% final concentration). Blanks (growth on a medium without carbohydrates) has been subtracted. Significant differences to B. longum subspecies suis ATCC 27533 were calculated using one-way ANOVA, followed by a Sidak's multiple comparison test performed on the different substrates (* p-value <0.05, ** p-value <0.01; *** p-value <0.001, **** p-value <0.0001). Values represent averages and standard deviation of biological triplicates.

FIG. 38—Cell density of selected B. longum subspecies suis, B. longum subspecies suillum and newly described B. longum clade strains after 48 h growth on glucose or on two commercially available scFOS fibers as sole carbon source (0.5% final concentration). Blanks (growth on a medium without carbohydrates) has been subtracted. Significant differences to B. longum subsp. suis ATCC 27533 were calculated using one-way ANOVA, followed by a Sidak's multiple comparison test performed on the different substrates (* p-value <0.05, ** p-value <0.01; *** p-value <0.001, **** p-value <0.0001). Values represent averages and standard deviation of biological triplicates.

FIG. 39—Glucose to scFOS growth ration of selected B. longum subspecies suis, B. longum subspecies suillum and newly described B. longum clade strains after 48 h growth on glucose or on two commercially available scFOS fibers as sole carbon source (0.5% final concentration). Ratios were calculated based on blanked values (growth on a medium without carbohydrates was subtracted). Significant differences to B. longum subspecies suis ATCC 27533 were calculated using one-way ANOVA, followed by a Sidak's multiple comparison test performed on the different substrate ratios (* p-value <0.05, ** p-value <0.01; *** p-value <0.001, **** p-value <0.0001). Values represent averages and standard deviation of biological triplicates.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “nutritional composition” refers to any kind of composition or formulation that provides a nutritional benefit to an individual and that may be safely consumed by a human or animal. Said nutritional composition may be in solid, semi-solid or liquid form and may comprise one or more macronutrients, micronutrients, food additives, water, etc. For instance, the nutritional composition may comprise the following macronutrients: a source of proteins, a source of lipids, a source of carbohydrates and any combination thereof. Furthermore, the nutritional composition may comprise the following micronutrients: vitamins, minerals, fiber, phytochemicals, antioxidants, prebiotics, probiotics, and any combination thereof. The composition may also contain food additives such as stabilizers (when provided in solid form) or emulsifiers (when provided in liquid form). The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or follow-on/follow-up formula or infant cereal product or any other formulation designed for infant or young child nutrition). Generally, a nutritional composition can be formulated to be taken enterally, orally, parenterally or intravenously, and it usually includes one of more nutrients selected from: a lipid or fat source, a protein source. and a carbohydrate source. Preferably, a nutritional composition is for oral use.

As used herein, the term “infant” means a human subject under the age of 12 months or an age-equivalent non-human animal.

As used herein, “young child” or “toddler” means a human subject aged between 12 months and 5 years of age.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide B. longum subspecies, particularly a new clade, that dominates during the transitional feeding period of an infant and various compositions and formulations thereof. This new clade is referred to herein as the B. longum transitional clade. Microorganisms of the B. longum transitional clade can be included in a synthetic composition and/or nutritional composition as a probiotic. The compositions can be used to assist and/or promote transitioning from a pure or partial milk-based diet to a diet of solid food in an infant or young child. Such synthetic compositions can be formulated, for example, as milk replacements, milk fortifiers, infant formulas, infant cereals and the like.

Embodiments disclosed herein also provide marker genes and/or signatures that characterize and/or uniquely identify microorganisms of the B. longum transitional clade. These marker genes and/or signatures can, for example, be used to assess a microbiome structure and identify modulating agents that can promote or limit the abundance of one or more microorganisms of the B. longum transitional clade within a microbiome, particularly a gastrointestinal microbiome.

Embodiments disclosed herein also provide genetically modified organisms that, while not being of a B. longum transitional clade, can be modified so as to have or express one or more B. longum transitional clade marker genes and/or signatures. These modified microorganisms can be included in compositions, such as a synthetic composition, with or without one or more microorganisms of the B. longum transitional clade, in a synthetic composition and/or nutritional composition as a probiotic. The compositions can be used to assist and/or promote transitioning from a pure or partial milk-based diet to a diet of solid food in an infant or young child. Such synthetic compositions can be formulated, for example, as milk replacements, milk fortifiers, infant formulas, infant cereals and the like.

B. longum Transitional Clade Microorganisms

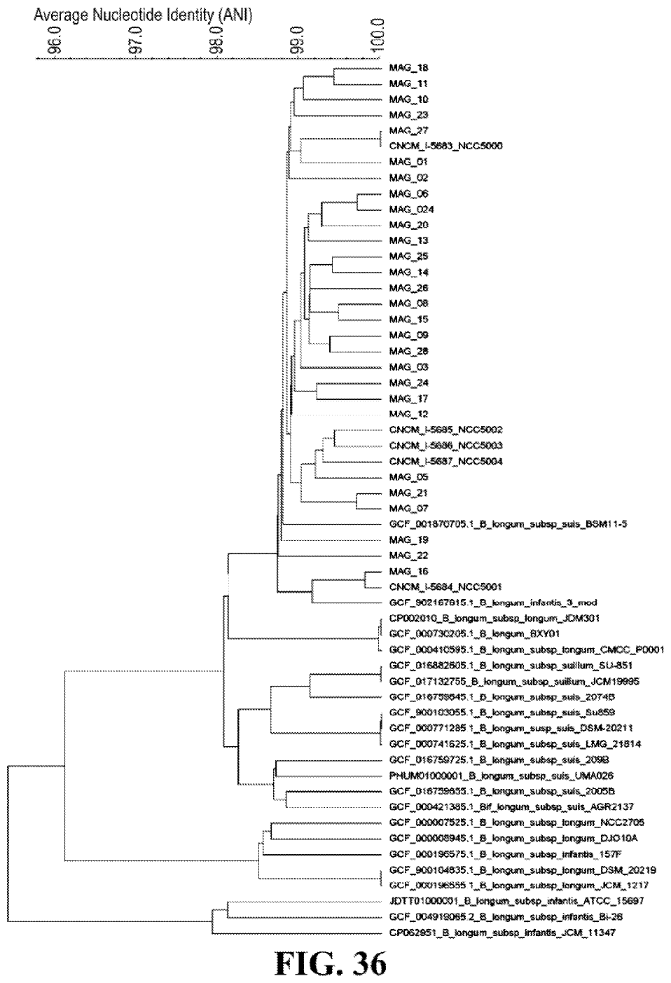

Described in several exemplary embodiments herein are Bifidobacterium longum spp. microorganisms of a clade that is present in a gastrointestinal microbiome of the transitional feeding period of mammals, particularly humans. B. longum microorganisms belonging to this clade are referred to herein as Bifidobacterium longum transitional (B. longum transitional) clade. As shown in the Working Examples herein, e.g., FIG. 36, the B. longum transitional clade microorganisms are greater in relative abundance during the transitional feeding period (e.g., weaning and as further defined elsewhere herein) than either B. longum subspecies infantis (B. infantis) as the relative abundance of B. longum subspecies infantis decreases at the beginning of the transitional feeding period until the end of the transitional feeding period when B. longum subspecies longum begins to increase in abundance.

The term “complementary feeding period”, “complementary period”, “transitional period”, “transitional feeding period”, which are used interchangeably herein, refers to the process or time period in which an infant or young child is moved (or transitioned), typically gradually over a period of time, from exclusive or partial milk feeding (such as breast milk feeding) to mix diet comprising breast milk (or replacers) and/or solid foods. This period depends on the individual infant and/or young child, but typically falls within the range from 4 months to 18 months of age, such as from about 6 to about 18 months, but in some instances can extend up to 24 months or more. This period is also referred to herein as the “weaning” period or “complementary feeding period”. For humans, the transition feeding period typically starts at age between 4 and 6 months of infant's age and is considered completed once the infant and/or young child is no longer fed with breast milk (or substitute infant formula), typically at 24 months. In one embodiment, the transition feeding period is between 4 and 24 months, for example 18 months of infant's and/or young child's age. This period is also referred to herein as the “weaning” period.

In some embodiments, a B. longum transitional clade microorganism has about 98.6%-100% Average Nucleotide Identity (ANI) with at least one strain selected from the group of: CNCM I-5683, CNCM I-5684, CNCM I-5685, CNCM I-5686 and CNCM I-5687. In some embodiments, a B. longum transitional clade microorganism has about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one strain selected from the group of: CNCM I-5683, CNCM I-5684, CNCM I-5685, CNCM I-5686 and CNCM I-5687. In some embodiments, a B. longum transitional clade microorganism has at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one strain selected from the group of: CNCM I-5683, CNCM I-5684, CNCM I-5685, CNCM I-5686 and CNCM I-5687. In some embodiments, a B. longum transitional clade microorganism has at least 98.6% ANI with at least one strain selected from the group of: CNCM I-5683, CNCM I-5684, CNCM I-5685, CNCM I-5686 and CNCM I-5687.

Average nucleotide identity (ANI) is a term of art that refers to a distance-based approach to delineate species based on pair-wise comparisons of their genome sequences and is an in silico alternative to the traditional DNA-DNA hybridization (DDH) techniques that have been used for phylogenetic definition of a species (Goris et al., 2007, “DNA-DNA hybridization values and their relationship to whole-genome sequence similarities”, Int. J. Syst. Evol. Microbiol. 57: 81-91). Based on DDH, strains with greater than 70% relatedness would be considered to belong to the same species (see e.g., Wayne et al., 1987, Report of the Ad-Hoc-Committee on Reconciliation of Approaches to Bacterial Systematics. Int J Syst Bacteriol 37: 463-464). ANI is similar to the aforementioned 70% DDH cutoff value and can be used for species delineation. ANI has been evaluated in multiple labs and has become the gold standard for species delineation (see e.g., Kim et al., 2014, “Towards a taxonomic coherence between average nucleotide identity and 16S rRNA gene sequence similarity for species demarcation of prokaryotes”, Int. J. Syst. Evol. Micr. 64: 346-351; Richter et al., 2009, “Shifting the genomic gold standard for the prokaryotic species definition”, P Nat Acad Sci USA 106: 19126-19131; and Chan et al., 2012, “Defining bacterial species in the genomic era: insights from the genus Acinetobacter”, Bmc. Microbiol. 12)).

The ANI of the shared genes between two strains is known to be a robust means to compare genetic relatedness among strains, and that ANI values of about 95% correspond to the 70% DNA-DNA hybridization standard for defining a species. See, e.g., Konstantinidis and Tiedje, Proc Natl Acad Sci USA, 102(7):2567-72 (2005); and Goris et al., Int Syst Evol Microbiol. 57(Pt 1):81-91 (2007). The ANI between two bacterial genomes is calculated from pair-wise comparisons of all sequences shared between any two strains and can be determined, for example, using any of a number of publicly available ANI tools, including but not limited to OrthoANI with usearch (Yoon et al. Antonie van Leeuwenhoek 110:1281-1286 (2017)); ANI Calculator, JSpecies (Richter and Rossello-Mora, Proc Nat Acad Sci USA 106:19126-19131 (2009)); and JSpeciesWS (Richter et al., Bioinformatics 32:929-931 (2016)). Other methods for determining the ANI of two genomes are known in the art. See, e.g., Konstantinidis, K. T. and Tiedje, J. M., Proc. Natl. Acad. Sci. U.S.A., 102: 2567-2572 (2005); and Varghese et al., Nucleic Acids Research, 43(14):6761-6771 (2015). In a particular embodiment, the ANI between two bacterial genomes can be determined, for example, by averaging the nucleotide identity of orthologous genes identified as bidirectional best hits (BBHs). Protein-coding genes of a first genome (Genome A) and second genome (Genome B) are compared at the nucleotide level using a similarity search tool, for example, NSimScan (Novichkov et al., Bioinformatics 32(15): 2380-23811 (2016). The results are then filtered to retain only the BBHs that display at least 70% sequence identity over at least 70% of the length of the shorter sequence in each BBH pair. The ANI of Genome A to Genome B is defined as the sum of the percent identity times the alignment length for all BBHs, divided by the sum of the lengths of the BBH genes. These and ANI determination techniques are known in the art and are described elsewhere herein.

In some embodiments, a B. longum transitional clade microorganism has about 98.6%-100% ANI with at least one genome sequence selected from the group of SEQ ID NOs: 1-4010. In some embodiments, a B. longum transitional clade microorganism has about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one genome sequence selected from the group of SEQ ID NOs: 1-4010. In some embodiments, a B. longum transitional clade microorganism has at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one genome sequence selected from the group of SEQ ID NOs: 1-4010.

In some embodiments, a B. longum transitional clade microorganism is characterized by the presence of all genes from Table 1. In some embodiments, a B. longum transitional clade microorganism is characterized by the presence of one or more genes from Table 1. In some embodiments, a B. longum transitional clade microorganism is characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1. In some embodiments, a B. longum transitional clade microorganism is characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1.

In some embodiments, a B. longum transitional clade microorganism is characterized by the presence of all genes from Table 2. In some embodiments, a B. longum transitional clade microorganism is characterized by the presence of one or more genes from Table 2. In some embodiments, a B. longum transitional clade microorganism is characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2. In some embodiments, the one or more B. longum transitional clade microorganisms included in the composition/formulation is characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2.

As used in this context herein, “presence . . . of a gene” refers to the inclusion of a gene or gene homologue in a genome of an organism (whether expressed or not) as determined by a suitable detection methodology and/or analysis. Suitable detection and/or analysis includes in silico analytics, genome sequencing, gene and/or protein expression analysis, or any combination thereof. One suitable method to resolve genomes of a microbiome to resolve microbiome structure and diversity of a microbiome is metagenomic analysis. Such an approach can be employed to survey individual microorganisms and their genomes present in the microbiome. Generally, metagenomics applies a suite of genomic sequencing technologies and bioinformatics tools to directly access the genetic content of entire communities of organisms. Generally, whole communities of microorganisms can be shot-gun sequenced in parallel and using in silico bioinformatics approaches, genomes can be resolved. The resulting genomes are referred to as Metagenome Assembled Genomes (MAGs). See e.g., Chen et al., 2020. Genome Res. 2020. 30: 315-333; Sangwan et al., Microbiome 4: 8.doi:10.1186/s40168-016-0154-5; Olson et al., Brief Bioinform 20: 1140-1150.doi:10.1093/bib/bbx098. From MAGs the presence (or absence) of a gene can be determined based on various bioinformatic approaches and tools, such as those that are alignment based and using reference gene sequences and/or genomes for comparison. It will be appreciated that the population can also be resolved by using other methods such as single cell sequencing techniques to determine the presence or absence of a given gene or genes. In one example embodiment, a gene is considered present if it is has a coverage of 90% in the MAGs analysis and has at least 95% shared sequence identity.

In some embodiments, the B. longum transitional clade microorganism is any one of CNCM I-5683, CNCM I-5684, CNCM I-5685, CNCM I-5686 and CNCM I-5687. In some embodiments, the B. longum transitional clade microorganism is any one of CNCM I-5683, CNCM I-5684, CNCM I-5685, CNCM I-5686 and CNCM I-5687 and any microorganism having a genome with about 100% ANI to any one of SEQ ID NOs: 1-4010.

In some embodiments, the composition/formulation includes one or more B. longum transitional clade microorganisms is not of the subspecies B. longum subspecies longum or B. longum subspecies infantis.

### Modified Microorganisms

In some embodiments, it can be advantageous to provide B. longum transitional clade microorganisms to a subject. In some instances, it may be advantageous to modify a non-B. longum transitional clade microorganism to contains and/or expresses one or more genes of a B. longum transitional clade signature and/or such that it comprises a B. longum transitional clade signature. This may be advantageous to generate a microorganism with the characteristics, function(s), and/or advantages of a B. longum transitional clade microorganism with the characteristics, function(s), and/or advantages of the background microorganism (i.e., the microorganism that is modified). Further, it will be appreciated that modification of a non-B. longum transitional clade microorganism can take place in vivo (e.g., via administration of a microbiome modulating agent and/or gene modifying agent) and thus change a microbiome structure such that it includes (where it previously did not) or it has more microorganisms that have a B. longum transitional clade signature and/or characteristics, functions, and/or advantages thereof (such as those that assist/promote transitioning from a milk-based diet to a solid and/or adult food diet). Other applications for the modified organisms described herein are discussed elsewhere herein and/or will be appreciated by those of ordinary skill in the art in view of the description herein.

In one example embodiment, a genetically modified microorganism is not of a Bifidobacterium longum transitional clade and is genetically modified to have modified expression of one or more genes from Table 1. In one example embodiments, the microorganism is not of a Bifidobacterium longum transitional clade and is genetically modified to have modified expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1.

In one example embodiment, the genetically modified microorganism is not of a Bifidobacterium longum transitional clade and is modified to have modified expression of one or more genes from Table 2. In one example embodiment, the microorganism is not of a Bifidobacterium longum transitional clade and is genetically modified to have modified expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2.

In some embodiments, the microorganism that is modified is a Bifidobacterium longum spp. that is not of a Bifidobacterium longum transitional clade. In one example embodiment, the microorganism that is modified is selected from the group consisting of a B. longum subspecies longum strain, is of a B. longum subspecies infantis strain, or a B. longum subspecies suis strain, or any combination thereof. In one example embodiment, the microorganism that is modified is selected from the group consisting of B. longum bb536; B. longum es 1; B. longum w11; B. longum NCC 3001; B. longum 1714; B. longum KACC 91563; B. longum subspecies longum SPM 1205, 1206, 1207, CECT 7347, MM-2; B. longum subspecies infantis ATCC 15697, Bifidobacterium longum subspecies infantis 35624, or any combination thereof.

As used in this context herein, “modified” broadly denotes a qualitative and/or quantitative alteration, change, or variation in that which is being modified. Where modification can be assessed quantitatively—for example, where modification comprises or consists of a change in a quantifiable variable such as a quantifiable property of a genome, transcriptome, proteome, epigenome, or other cell property or where a quantifiable variable provides a suitable surrogate for the modification (e.g., genotype, gene and/or protein expression, methylation, etc.)—modification specifically encompasses an increase (e.g., activation) or decrease (e.g., inhibition) in the measured variable. “Increase” in this context encompasses modifications that provide any measurable presence or activity where there was not any prior to the modification. Similarly, “decrease” in this context encompasses the elimination of any measurable activity or presence after modification. Thus, the term “modified” encompasses the change associated with introduction of a feature not previously present as well as the removal of a feature such that it is no longer present after the modification. For example, insertion of an exogenous gene can result in modified expression (e.g., an increase in expression) of that gene relative to the unmodified state. Similarly, deletion or disruption of a gene can result in modified expression (e.g., decreased expression) of that gene relative to the unmodified state. The term encompasses any extent of such modification, e.g., any extent of such increase or decrease, and may more particularly refer to statistically significant increase or decrease in the measured variable. By means of example, in embodiments modification may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In embodiments, “modified” encompasses a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In some embodiments, modification may be binary—e.g., presence or absence of a feature. For example, when a genome is modified, the modification may result in the inclusion (and/or deletion) of one or more nucleotides and thus the modification may be measured or evaluated in a binary approach (e.g., yes/no, or present/absent, A or T, G or C, etc.).

“Modified” encompasses a deviation of a first value from a second value in any direction (e.g., increase: first value >second value; or decrease: first value <second value) and any extent of alteration. For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values (as expressed, for example, by standard deviation or standard error, or by a predetermined multiple thereof, e.g., ±1×SD or ±2×SD or +3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

As used herein in, in the context of polynucleotide or gene “expression” refers to the process by which polynucleotides are transcribed into RNA transcripts. In the context of mRNA and other translated RNA species, “expression” refers to the process or processes by which the transcribed RNA is subsequently translated into peptides, polypeptides, or proteins. In some instances, “expression” reflects the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation of “expression” in these various contexts to the underlying biological mechanisms.

As used herein, “modified expression” generally denotes an alteration (increase or decrease) in the expression of a polynucleotide (e.g., a DNA or RNA) or protein according to the definitions of “modified” and “expression” provided herein.

### Bacteria Genome Editing Methods

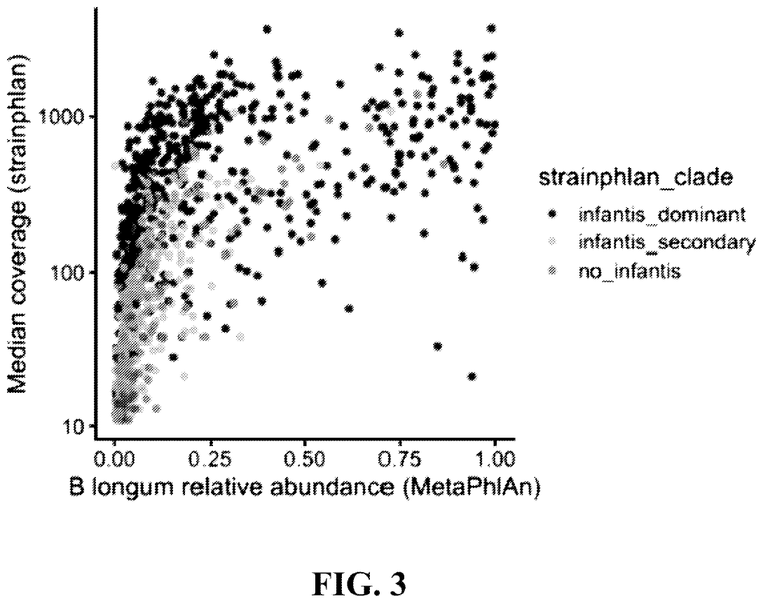

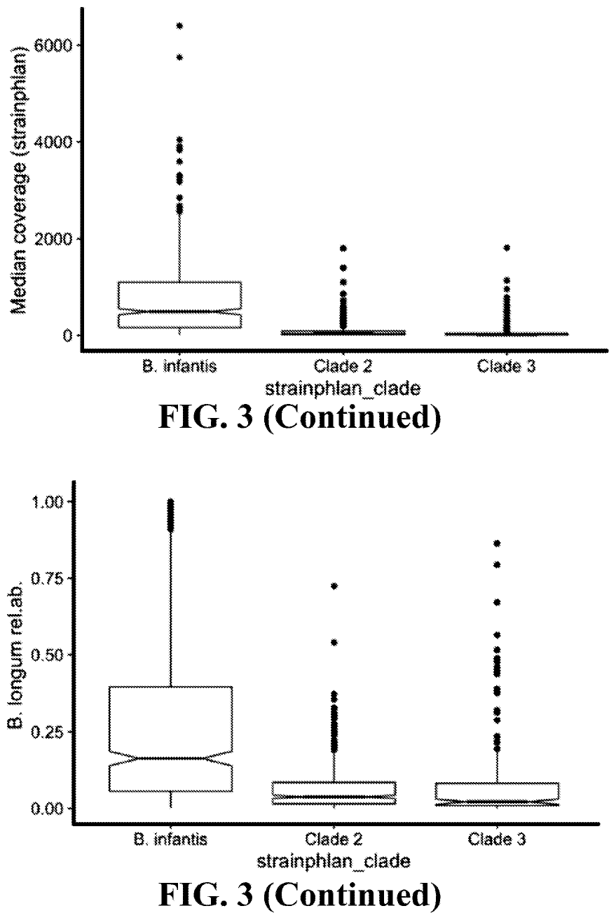

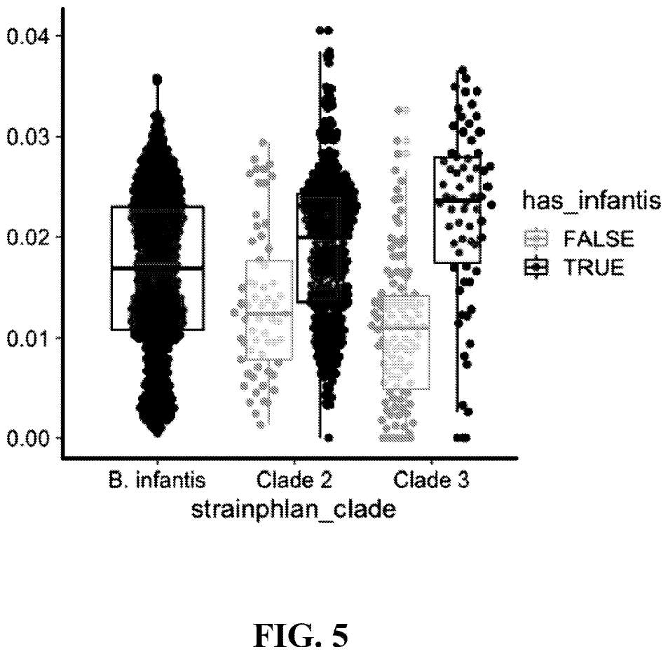

Modified bacteria may be generated using known bacterial genome editing methods. For example, plasmid-based gene knock-out and knock-in approaches may be used to remove or insert genes into bacterial genomes, respectively. Lambda bacteriophage-based approaches, e.g., k Red systems, may also be used to facilitate recombination-mediated genetic engineering. Alternatively, Group II intron and/or Group II intron, Cre/lox combinations may be used to facilitate both gene knock-out, gene knock-in and other large-scale genomic modifications. CRISPR-Cas systems have also been adapted to for bacteria genome editing. An overview of bacterial genome engineering systems is discussed in Nakashima and Miyazaki, International Journal of Molecular Sciences 2014, 15, 2773-2793, particularly Table 2, which is specifically incorporated herein by reference. CRISPR-Cas based systems for bacterial genome editing, and variations thereof, are further discussed in Arroyo-Olarte et al. Microorganisms, 2021, 9, 844, particularly FIGS. 3 and 5, which are specifically incorporated herein by reference.

B. longum Transitional Clade Compositions

Described in several exemplary embodiments herein are compositions and formulations that can included one or more of the B. longum transitional clade microorganisms described and demonstrated elsewhere herein. In some embodiments, the compositions and/or formulation include at least one, at least two, or more B. longum transitional clade strains. In some embodiments the compositions are synthetic compositions and/or nutritional compositions.

In some embodiments, the compositions include one or more modified microorganisms as described in greater detail elsewhere herein.

In some embodiments, the one or more B. longum transitional clade microorganisms included in the composition have about 98.6%-100% Average Nucleotide Identity (ANI) with at least one strain selected in the group of: CNCM I-5683, CNCM I-5684, CNCM I-5685, CNCM I-5686 and CNCM I-5687. In some embodiments, the one or more B. longum transitional clade microorganisms included in the composition have about 98.6%, 98.7%, 98.8%, 98.9%, 99%, 99.1%, 99.2%, 99.3%, 99.4%, 99.5%, 99.6%, 99.7%, 99.8%, 99.9%, or 100% Average Nucleotide Identity (ANI) with at least one strain selected in the group of: CNCM I-5683, CNCM I-5684, CNCM I-5685, CNCM I-5686 and CNCM I-5687. In some embodiments, the one or more B. longum transitional clade microorganisms have at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one strain selected in the group of: CNCM I-5683, CNCM I-5684, CNCM I-5685, CNCM I-5686 and CNCM I-5687. In some embodiments, the one or more B. longum transitional clade microorganisms included in the composition have at least 98.6% Average Nucleotide Identity (ANI) with at least one strain selected in the group of: CNCM I-5683, CNCM I-5684, CNCM I-5685, CNCM I-5686 and CNCM I-5687.

In some embodiments, the one or more B. longum transitional clade microorganisms included in the composition is characterized by the presence of all genes from Table 1. In some embodiments, the one or more B. longum transitional clade microorganisms included in the composition is characterized by the presence of one or more genes from Table 1. In some embodiments, the one or more B. longum transitional clade microorganisms included in the composition/formulation is characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1. In some embodiments, the one or more B. longum transitional clade microorganisms included in the composition is characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1.

In some embodiments, the one or more B. longum transitional clade microorganisms included in the composition is characterized by the presence of all genes from Table 2. In some embodiments, the one or more B. longum transitional clade microorganisms included in the composition is characterized by the presence of one or more genes from Table 2. In some embodiments, the one or more B. longum transitional clade microorganisms included in the composition is characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2. In some embodiments, the one or more B. longum transitional clade microorganisms included in the composition is characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2.

In some embodiments, the composition includes one or more B. longum transitional clade microorganisms having about 98.6%-100% ANI with at least one genome sequence selected from the group of SEQ ID NOs: 1-4010. In some embodiments, the composition includes one or more B. longum transitional clade microorganisms having about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one genome sequence selected from the group of SEQ ID NOs: 1-4010. In some embodiments, the composition includes one or more B. longum transitional clade microorganisms having at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one genome sequence selected from the group of SEQ ID NOs: 1-4010.

In some embodiments, the composition includes one or more B. longum transitional microorganisms is not of the subspecies B. longum subspecies longum or B. longum subspecies infantis.

In some embodiments, the composition includes one or more modified microorganisms having a B. longum transitional clade signature. Exemplary modified microorganisms are described in greater detail elsewhere herein.

The one or more B. longum transitional clade microorganisms and/or modified microorganisms can each be included in the composition an amount from about 10¹ to 10¹⁸ cfu (colony forming units), such as 10² to 10¹⁵, 10³ to 10¹², 10⁵ to 10¹², 10⁶ to 10¹², 10⁷ to 10¹², or 10⁸ to 10¹⁰ of each strain per g of composition or formulation on a dry weight basis or per mL of composition on a volume basis. In some embodiments, the compositions include about 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, or about 10¹⁸ cfu of one or more B. longum transitional clade microorganisms and/or modified microorganisms as described in greater detail elsewhere herein per g of composition or formulation on a dry weight basis or per mL of composition on a volume basis. In some embodiments, the composition includes about 10¹, to about 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, or about 10¹⁸ cfu of one or more B. longum transitional clade microorganisms and/or modified microorganism as described elsewhere herein per g of composition on a dry weight basis or per mL of composition on a volume basis.

In some embodiments, one or more of the one or more B. longum transitional clade microorganisms and/or modified microorganisms are viable. In some embodiments, one or more of the one or more B. longum transitional clade microorganisms and/or modified microorganisms are non-replicating or inactivated. In some embodiments, the composition contains both viable B. longum transitional clade microorganisms and/or modified microorganisms and inactivated B. longum transitional clade microorganisms and/or modified microorganisms.

In some embodiments, the composition includes a milk or milk-derived product. The milk or milk-derived product can be a milk produced from a mammal or a component/product derived therefrom. Mammalian milks include, without limitation, human breast milk, cows' milk, human milk, sheep milk, goat milk, horse milk, camel milk, and combinations thereof. In some embodiments, the milk or milk-derived product is a plant milk or plant milk-derived product. The term “plant milk” is a term of art that includes liquid milk-like substances produced from plants (e.g., soy milk, almond milk, cashew milk, coconut milk, rice milk, and combinations thereof).

The compositions that can be formulated appropriate for infant and/or young child use. In addition of the one or more B. longum transitional clade microorganisms the compositions can include one or more other components, such as macro and micro-nutrients (e.g., fats, fiber, carbohydrates, vitamins and minerals), inert and/or other functional components such as fillers, emulsifiers, etc. Other beneficial components can be included, such as additional probiotic components. Such compositions can be formulated for use in an infant and/or young child. Such compositions and formulations are further described in greater detail elsewhere herein.

In some embodiments, the composition including one or more B. longum transitional clade microorganisms and/or modified microorganisms is a synthetic composition. The expression “synthetic composition” means a mixture obtained by chemical and/or biological methods and techniques. In some embodiments, the synthetic composition which can biologically, nutritionally, and/or chemically equivalent or identical to the mixture naturally occurring in mammalian milks or a component thereof (e.g., the fat profile or protein or amino acid profile). The term “equivalent” as used in this context refer to a composition that is not identical but provides the same function and/or provides the same nutritional profile or make up as that occurring in a reference natural mammalian milk. For example, a synthetic composition may have a different amino acid profile than that occurring in natural human milk but still provides the same protein content, fat content and carbohydrate content and provides the same minimum nutritional requirements for an e.g., infant and/or young child that reference natural human milk would. Thus, although they are not identical, they are equivalent nutritionally. This principle can be expanded by one of ordinary skill in the art to other characteristics of a synthetic solution use objective measurement techniques known in the art to determine, against a reference sample, if despite the difference in chemical make-up, they would be considered the same with respect to that characteristic or function.

In some embodiments, and as is also described elsewhere herein, the composition includes one or more milk-based ingredients. As used herein, expression “milk-based ingredient” or “milk-based ingredients” identifies carbohydrate containing ingredients derived from mammal milk for example cow, goat and/or buffalo or mixtures thereof. Non limiting examples of such ingredients comprise: fresh milk, concentrated milk, powder milk, whole milk, skimmed and/or semi-skimmed milk. As it will be apparent to the skilled person, milk-based ingredients according to the present invention may bring additional nutrients beyond carbohydrates to the complementary nutritional composition, such as for example proteins and fats.

In some embodiments, the composition is a transitional nutritional composition. Transitional nutritional compositions are described in greater detail elsewhere herein. In some embodiments, the transitional nutritional composition or other synthetic composition described herein provides to the infant and/or young child assuming it at least 10%, for example 20%, or 30%, or 40%, or 50%, or 60% or 70%, or 80%, or 90%, or 100% (i.e., the total) of the total daily caloric intake. Typically, the caloric density as well as the amounts and kinds of proteins, carbohydrates and lipids present in the composition should be carefully adjusted to the needs of the infant and are dependent on the infant and/or young child stage of development and age. Such nutritional calculation and/or balancing will be within the purview of one of ordinary skill in the art in view of the present disclosure.

### Proteins

In some embodiments the compositions and/or formulations can include one or more proteins and/or protein sources. The protein can be in an amount ranging from 1.6 to 3 g per 100 kcal. In some embodiments, the protein amount can be between 2.4 and 4 g/100 kcal or more than 3.6 g/100 kcal. Such formulations can be advantageous particularly when the infant is a premature infant. In some embodiments, the protein amount can be below 2.0 g per 100 kcal, e.g., between 1.8 to 2 g/100 kcal, or in an amount below 1.8 g per 100 kcal.

The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e., more than 50% of proteins are coming from whey proteins, such as 60% or 70%).

The proteins may be intact or hydrolyzed or a mixture of intact and hydrolyzed proteins. By the term “intact” is meant that the main part of the proteins is intact, i.e., the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolyzed” means in the context of the present invention a protein which has been hydrolyzed or broken down into its component amino acids.

The proteins can be either fully or partially hydrolyzed. It may be desirable to supply partially hydrolyzed proteins (degree of hydrolysis between 2 and 20%), for example for infants and/or young children believed to be at risk of developing cow's milk allergy. If hydrolyzed proteins are required, the hydrolysis process may be carried out as desired and as is known in the art. For example, whey protein hydrolysates may be prepared by enzymatically hydrolyzing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example, about 7% by weight of lysine which greatly improves the nutritional quality of the protein source.

In some embodiments, at least 70% of the proteins are hydrolyzed, preferably at least 80% of the proteins are hydrolyzed, such as at least 85% of the proteins are hydrolyzed, even more preferably at least 90% of the proteins are hydrolyzed, such as at least 95% of the proteins are hydrolyzed, particularly at least 98% of the proteins are hydrolyzed. In a particular embodiment, 100% of the proteins are hydrolyzed.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90.

In a particular embodiment the nutritional composition according to the invention is a hypoallergenic composition. In another particular embodiment the composition according to the invention is a hypoallergenic nutritional composition.

### Fats and Lipids

In some embodiments the compositions and/or formulations can include one or more lipids and/or fats and/or lipid and/or fat sources. The expression “fat” or “fat source” or “lipid” or “lipid source” or “fats” in the context of the present invention indicates an edible solid or liquid fat or mixtures thereof. Not limiting categories of fats are those from animal, fish or vegetable origins. Non-limiting examples of fats which can be included in various embodiments of the compositions and formulations herein include fish oil, cocoa butter, cocoa butter equivalents (CBE), cocoa butter substitutes (CBS), vegetable oils (for example rapeseed oil, palm oil, corn oil, soy oil, coconut oil and/or sunflower oil) and butter oils amongst others.

Inclusion of a fat/lipid or source thereof in a composition or formulation described herein is particularly relevant if the composition and/or formulation is an infant formula and/or intended for infant use. In this case, the lipid/fat source may be any lipid or fat which is suitable for use in infant formulae. Some suitable fat sources include palm oil, high oleic sunflower oil and high oleic safflower oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example, about 8:1 to about 10:1.

In some embodiments, the fat/lipid includes one or more fatty acids. In some embodiments the one or more fatty acid(s) is Docosahexaenoic acid (DHA). In some embodiments, the amount included is at least the minimum recommended amount based on daily dosage or an amount calculated to deliver at least the minimum recommended daily dosage when administered as intended.

### Carbohydrates

In some embodiments the compositions and/or formulations can include one or more carbohydrates and/or carbohydrate sources. This is particularly preferable in the case where the composition and/or formulation is an infant formula. In some embodiments, the synthetic formulation can include any carbohydrate source conventionally found in infant formulae such as lactose, sucrose, saccharose, maltodextrin, starch and mixtures thereof may be used although one of the preferred sources of carbohydrates is lactose. Carbohydrates include, sugars (simplest carbohydrates), oligosaccharides, and polysaccharides (e.g., starch and dextrin).

### Sugars/Oligosaccharides

In some embodiments the compositions and/or formulations can include one or more sugars and/or oligosaccharides and/or sugar and/or oligosaccharide sources.

### Sugars

In some embodiments, compositions and/or formulations herein include one or more sugars and/or sugar sources. As used herein the term “sugar” refers to mon- and disaccharides. Sugars can be natural or synthetic. Sugars that can be included in various embodiments of the compositions and/or formulations herein include, but are not limited to, sucrose, glucose, fructose, maltose, arabinose, fucose, galactose, mannose, ribose, dextrose, trehalose, and xylose. Sugar can come from a variety of sources (i.e., a sugar source) including, but not limited to, agave syrup, bee honey, beets, tree sap (e.g., maple or other trees (e.g., birch, palm, trees, carob pods, etc.), cane, coconut, corn, date, various fruits (e.g., grapes, apple, orange, etc.), sorghum, more complex carbohydrates (e.g., oligosaccharides and polysaccharides) etc. As used herein, the term “fruit” or “fruits” indicates ingredients derived from fruit such as for example fresh fruit, fruit paste, dried fruit, fruit extracts and/or centrifugates. Mixtures of such ingredients are also comprised within the scope of the terms above mentioned. Non-limiting examples of fruit that can be included in some embodiments of the compositions and formulations described herein are: grape, apple, apricot, banana, cherry, pear, strawberry, mango, orange, and peach.

The quantification of mono- and disaccharides in a composition described herein can be completed by weighing a 1-3±0.001 gram sample into a 100 mL volumetric flask and 60 milliliters of demineralized water were added. Mono- and disaccharides contained in the samples are extracted by placing the flasks into a 70° C. water bath for 20 min with constant agitation. Samples are cooled to room temperature and more demineralized water is added to make up the mark on each volumetric flask, stoppers placed, and closed flasks were shaken vigorously. Samples are then filtered through folded filter paper (No 597, 150 mm ø) and through a 0.2 m HPLC-filter before injection (25 mm ø, 8825-P-2 Infochroma AG). A solution containing monomeric glucose, dimeric lactose, sucrose, maltose, isomaltose, and maltooligosaccharides having a degree of polymerization ranging from 3-7 is prepared as standard for peak identification and saccharide quantification.

### Sugar Alcohols

In some embodiments, the composition and/or formulation includes one or more sugar alcohols. As used herein, “sugar alcohol” refers to organic compounds, having the general formula of HOCH₂(CHOH)ₙCH₂OH. Generally, sugar alcohols are not cyclic but can be. Sugar alcohols can be derived from sugars, but not necessarily. The sugar alcohol(s) can be natural or synthetic. Sugar alcohols include, without limitation, inositol, xylitol, allulose, erythritol, sorbitol, mannitol, maltitol, lactitol, isomalt, glycerol, and various hydrogenated starch hydrolysates.

### Oligosaccharides

In some embodiments, the compositions and/or formulations described herein include one or more oligosaccharides. The term of art “oligosaccharide” refers to a carbohydrate that has greater than 2 but relatively few monosaccharide units (typically 3, 4, 5, or 6). Exemplary oligosaccharides include, but are not limited to, fructo-oligosaccharides, galacto-oligosaccharides (raffinose, stachyose, verbascose), maltotriose, gentio-oligosaccharides, gluco-oligosaccharides, milk oligosaccharides (e.g., those present in secretions from mammary glands), isomalto-oligosaccharides, lactosucrose, mannan-oligosaccharides, melibiose-derived oligosaccharides, pectic oligosaccharides, xylo-oligosaccharides.

In some embodiments, the oligosaccharide(s) is or includes a human milk oligosaccharide (HMO). In some embodiments, the oligosaccharide(s) is or includes a bovine milk oligosaccharide (BMO). In some embodiments, the oligosaccharide(s) is or includes one or more oligosaccharides from sheep, goat, or other animal source. It will be appreciated that oligosaccharides can be included as sweeteners, energy source, and/or as fibers (digestible or indigestible). The term “HMO” or “HMOs” refers to human milk oligosaccharide(s). These carbohydrates are highly resistant to enzymatic hydrolysis, indicating that they may display essential functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk-over 130 such structures have been identified so far. Almost all of them have a lactose moiety at their reducing end while sialic acid and/or fucose (when present) occupy terminal positions at the non-reducing ends. The HMOs can be acidic (e.g., charged sialic acid containing oligosaccharide) or neutral (e.g., fucosylated oligosaccharide). In some embodiments, the HMOs are included in the composition in amounts as set forth in International Application Publication No. WO2012156273. In some embodiments, the HMOs can be present at an adaptive level of HMOs so as to form an age tailored formulation, such as those set forth in e.g., U.S. Pat. No. 10,820,616.

In some embodiments, the compositions described herein include HMOs in a total amount of from 1000 to 10000 mg/L, preferably from 1500 to 8000 mg/L, more preferably from 2000 to 5000 mg/L, even more preferably from 3000 to 4000 mg/L, even more preferably from 3590 to 3673 mg/L, and most preferably in an amount of 3632 mg/L of composition. In some embodiments, the compositions described herein include HMOs in an amount of from 1500 to 3500 mg/L, preferably from 2000 to 3000 mg/L, more preferably from 2558 to 2602 mg/L, and most preferably in an amount of 2580 mg/L of composition. In some embodiments, the compositions described herein include HMOs in an amount of from 500 to 2500 mg/L, preferably from 1500 to 2000 mg/L, more preferably from 1863 to 1902 mg/L, and most preferably in an amount of 1883 mg/L of composition.

### Fucosylated Oligosaccharides

In some embodiments the oligosaccharide(s) is or includes one or more fucosylated oligosaccharides. A “fucosylated oligosaccharide” is an oligosaccharide having a fucose residue. It has a neutral nature. Some examples are 2-FL (2′-fucosyllactose), 3-FL (3-fucosyllactose), difucosyllactose, lacto-N-fucopentaose (e.g., lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

The expressions “fucosylated oligosaccharides comprising a 2′-fucosyl-epitope” and “2-fucosylated oligosaccharides” encompass fucosylated oligosaccharides with a certain homology of form since they contain a 2′-fucosyl-epitope, therefore a certain homology of function can be expected.

In some particular embodiments the fucosylated oligosaccharide comprises a 2′-fucosyl-epitope. It can be for example selected from the list comprising 2′-fucosyllactose, difucosyllactose, lacto-N-fucopentaose, lacto-N-fucohexaose, lacto-N-difucohexaose, fucosyllacto-N-hexaose, fucosyllacto-N-neohexaose, difucosyllacto-N-hexaose difuco-lacto-N-neohexaose, difucosyllacto-N-neohexaose, fucosyl-para-Lacto-N-hexaose and any combination thereof.

In some embodiments, the synthetic formulation includes a 2′-fucosyllactose (or 2FL, or 2′FL, or 2-FL or 2′-FL). In a particular embodiment, there is no other type of fucosylated oligosaccharide than 2′-fucosyllactose, i.e., the nutritional composition of the invention comprises only 2′-fucosyllactose as fucosylated oligosaccharide.

The fucosylated oligosaccharide(s) may be isolated by chromatography or filtration technology from a natural source such as animal milks. Alternatively, it may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerization (DP), from DP=1 onwards. Alternatively, fucosylated oligosaccharides may be produced by chemical synthesis from lactose and free fucose. Fucosylated oligosaccharides are also available for example from Kyowa, Hakko, Kogyo of Japan.

The fucosylated oligosaccharide(s) can be present in the nutritional composition according to the present invention in a total amount of 0.2-3 g/L, for example 0.5-2 g/L or 0.75-1.65 g/L of the composition. In some embodiments, the fucosylated oligosaccharide(s) may be in a total amount of 0.8-1.5 g/L of the composition, such as 0.85-1.3 g/L or 0.9-1.25 g/L or 0.9-1.1 g/L or 1-1.25 g/L or 1.05-1.25 g/L of the composition. In a particular embodiment, the fucosylated oligosaccharide(s) is/are in a total amount of 1 g/L of the composition. In another particular embodiment, the fucosylated oligosaccharide(s) is/are in a total amount of 1.24 g/L of the composition. The fucosylated oligosaccharide(s) can be present in the nutritional composition in a total amount of 0.13-2.1 g/100 g, for example 0.34-1.4 g/100 g or 0.52-1.15 g/100 g of composition on a dry weight basis. The fucosylated oligosaccharide(s) may be in a total amount of 0.55-1.05 g/100 g of the composition, such as 0.59-0.9 g/100 g, or 0.62-0.87 g/100 g or 0.62-0.77 g/100 g or 0.69-0.87 g/100 g or 0.73-0.87 g/100 g of the composition. In a particular embodiment, the fucosylated oligosaccharide(s) is/are in a total amount of 0.69 g/100 g of the composition. In another particular embodiment, the fucosylated oligosaccharide(s) is/are in a total amount of 0.86 g/100 g of the composition.

### N-Acetylated Oligosaccharide(s)

In some embodiments the oligosaccharide(s) is or includes one or more N-acetylated oligosaccharides. The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) or any combination thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

In some specific embodiments, the synthetic formulation can include at least one the N-acetylated oligosaccharide. There can be one or several types of N-acetylated oligosaccharide. The N-acetylated oligosaccharide(s) can be for example lacto-N-tetraose (LNT), lacto-N-neotetraose (LNnT) or any combination thereof. In some particular embodiments the N-acetylated oligosaccharide is lacto-N-neotetraose (LNnT), para-lacto-N-neohexaose (para-LNnH) or any combination thereof. In some particular embodiments the N-acetylated oligosaccharide is LNnT. In some particular embodiments the N-acetylated oligosaccharide is LNT. In some other particular embodiments, the N-acetylated oligosaccharide is a mixture of LNT and LNnT. In some particular embodiments the composition comprises both LNT and LNnT in a ratio LNT:LNnT between 5:1 and 1:2, or from 2:1 to 1:1, or from 2:1.2 to 2:1.6. The N-acetylated oligosaccharide(s) may be synthesised chemically by enzymatic transfer of saccharide units from donor moieties to acceptor moieties using glycosyltransferases as described for example in U.S. Pat. No. 5,288,637 and WO 96/10086. Alternatively, LNT and LNnT may be prepared by chemical conversion of Keto-hexoses (e.g., fructose) either free or bound to an oligosaccharide (e.g., lactulose) into N-acetylhexosamine or an N-acetylhexosamine-containing oligosaccharide as described in Wrodnigg, T. M.; Stutz, A. E. (1999) Angew. Chem. Int. Ed. 38:827-828. N-acetyl-lactosamine produced in this way may then be transferred to lactose as the acceptor moiety.

The N-acetylated oligosaccharide(s) can be present in the nutritional composition according to the present invention in a total amount of 0.1-2 g/L, for example 0.3-1 g/L or 0.45-0.85 g/L of the composition.

In some embodiments, the N-acetylated oligosaccharide(s) may be in a total amount of 0.5-0.8 g/L of the composition, such as 0.5-0.75 g/L or 0.5-0.7 g/L of the composition. In a particular embodiment, the N-acetylated oligosaccharide(s) is/are in a total amount of 0.5 g/L of the composition. In another particular embodiment, the N-acetylated oligosaccharide(s) is/are in a total amount of 0.63 g/L of the composition.

The N-acetylated oligosaccharide(s) can be present in the nutritional composition in a total amount of 0.06-1.4 g/100 g, for example 0.2-0.7 g/100 g or 0.31-0.59 g/100 g of composition on a dry weight basis, The N-acetylated oligosaccharide(s) may be in a total amount of 0.35-0.56 g/100 g of composition, such as 0.35-0.52 g/100 g or 0.35-0.49 g/100 g. In a particular embodiment, the N-acetylated oligosaccharide(s) is/are in a total amount of 0.35 g/100 g of the composition. In another particular embodiment, the N-acetylated oligosaccharide(s) is/are in a total amount of 0.44 g/100 g of the composition.

In some other particular embodiments, the synthetic formulation does not comprise any N-acetylated oligosaccharides

In some embodiments, the synthetic formulation comprises both 2′-fucosyllactose (2-FL) and lacto-N-neotetraose (LNnT). In another specific embodiment, the nutritional composition of the present invention comprises an oligosaccharide mixture that consists of 2′-fucosyllactose (2-FL) and lacto-N-neotetraose (LNnT). In other words, the nutritional composition of the invention comprises only 2′-fucosyllactose (2-FL) as fucosylated oligosaccharide and only lacto-N-neotetraose (LNnT) as N-acetylated oligosaccharide.

The synthetic formulation can include fucosylated oligosaccharide(s) and N-acetylated oligosaccharide(s) in a ratio fucosylated oligosaccharide(s):the N-acetylated oligosaccharide(s) of from 2:0.065 to 2:20 such as from 2:0.3 to 2:4, for example 2:0.54 to 2:2.26, or 2:0.76-2:1.8 or 2:0.8-2:1.4. In a particularly advantageous embodiment, this ratio is 2:1 or around 2:1.

### Sialylated Oligosaccharides

In some embodiments the oligosaccharide(s) is or includes one or more sialylated oligosaccharides. A “sialylated oligosaccharide” is a charged sialic acid containing oligosaccharide, i.e., an oligosaccharide having a sialic acid residue. It has an acidic nature. Some examples are 3-SL (3′ sialyllactose) and 6-SL (6′ sialyllactose). The sialylated oligosaccharide(s) can be selected from the group comprising 3′ sialyllactose (3-SL), 6′ sialyllactose (6-SL), and any combination thereof. In some embodiments of the invention the composition comprises 3-SL and 6-SL. In some particular embodiments the ratio between 3′-sialyllactose (3-SL) and 6′-sialyllactose (6-SL) can be in the range between 5:1 and 1:10, or from 3:1 and 1:1, or from 1:1 to 1:10. In some specific embodiments the sialylated oligosaccharide of the composition is 6′ sialyllactose (6-SL).

The sialylated oligosaccharide(s) may be isolated by chromatographic or filtration technology from a natural source such as animal milks. Alternatively, they may be produced by biotechnological means using specific sialyltransferases or sialidases, neuraminidases, either by an enzyme-based fermentation technology (recombinant or natural enzymes), by chemical synthesis or by a microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP), from DP=1 onwards. Alternatively, sialyllactoses may be produced by chemical synthesis from lactose and free N′-acetylneuraminic acid (sialic acid). Sialyllactoses are also commercially available for example from Kyowa Hakko Kogyo of Japan.

In particular examples, the synthetic formulation includes from 0.05 to 5 g/L of sialylated oligosaccharide(s), or from 0.1 to 4 g/L, or from 0.3 to 2 g/L, or from 0.4 to 1.5 g/L, or from 0.4 to 1 g/L, for example 0.5 or 0.9 g/L of sialylated oligosaccharide(s). In some particular embodiments, the synthetic formulation includes from 0.8 to 1.7 g/l of sialylated oligosaccharide(s).

The synthetic formulation includes can contain from 0.03 to 3.5 g of sialylated oligosaccharide(s) per 100 g of composition on a dry weight basis, e.g., from 0.1 to 2 g or from 0.2 to 1 g or from 0.3 to 0.6 g of sialylated oligosaccharide(s) per 100 g of composition on a dry weight basis.

The synthetic formulation can include sialylated oligosaccharide(s) in an amount of below 0.1 g/100 g of composition on a dry weight basis.

In some particular embodiments, the synthetic formulation does not contain any sialylated oligosaccharide(s).

In some other particular embodiments of the present invention, the nutritional composition does not contain any galacto-oligosaccharides (GOS).

### Oligosaccharide Precursors

In some embodiments, the synthetic formulation optionally also includes at least one precursor of oligosaccharide. There can be one or several precursor(s) of oligosaccharide. For example, the precursor of human milk oligosaccharide is sialic acid, fucose or a mixture thereof. In some particular embodiments the composition comprises sialic acid.

In particular examples the composition comprises from 0 to 3 g/L of precursor(s) of oligosaccharide, or from 0 to 2 g/L, or from 0 to 1 g/L, or from 0 to 0.7 g/L, or from 0 to 0.5 g/L or from 0 to 0.3 g/L, or from 0 to 0.2 g/L of precursor(s) of oligosaccharide.

The composition according to the invention can contain from 0 to 2.1 g of precursor(s) of oligosaccharide per 100 g of composition on a dry weight basis, e.g., from 0 to 1.5 g or from 0 to 0.8 g or from 0 to 0.15 g of precursor(s) of oligosaccharide per 100 g of composition on a dry weight basis.

### Fiber

In some embodiments, the compositions and/or formulations described herein can include a fiber and/or fiber source(s). It will be appreciated that some fibers are carbohydrates that are relatively indigestible by a human or animal. Such fibers are also discussed in relation to carbohydrates herein. In some embodiments, the fiber can be digested by one or microorganisms present in the composition and/or formulation and/or within one or more regions in the gastrointestinal tract within an organism, such as a human or non-human animal. As used herein, the expressions “fiber” or “fibers” or “dietary fiber” or “dietary fibers” within the context of the present invention indicate the indigestible portion, in small intestine, of food derived from plants which comprises two main components: soluble fiber, which dissolves in water and insoluble fiber. Mixtures of fibers are comprised within the scope of the terms above mentioned. Soluble fiber is readily fermented in the colon into gases and physiologically active byproducts and can be prebiotic and viscous. Insoluble fiber does not dissolve in water, is metabolically inert and provides bulking, or it can be prebiotic and metabolically ferment in the large intestine. Chemically, dietary fiber consists of non-starch polysaccharides such as arabinoxylans, cellulose, and many other plant components such as resistant starch, resistant dextrins, inulin, lignin, chitins, pectins, beta-glucans, and oligosaccharides. Non-limiting examples of dietary fibers are: prebiotic fibers such as Fructo-oligosaccharides (FOS), inulin, galacto-oligosaccharides (GOS), fruit fiber, vegetable fiber, cereal fiber, resistant starch such as high amylose corn starch. As fibers are not digestible, they do not contain available carbohydrates and on this basis, they do not contribute to the GI or GL of the composition they're part of.

As used herein, “added fiber” or “added dietary fiber” indicates an ingredient mainly or totally constituted by fiber which is added to the complementary nutritional composition and whose content in fiber contributes to the total fiber content of the composition. The total fiber content of the complementary nutritional composition is provided by the sum of amount of fiber naturally present in ingredients used in the recipe (for example from whole grain cereal flour) plus amount of added fiber.

### Prebiotics

In some embodiments, the composition can include one or more prebiotics. As used herein, “prebiotic” means non-digestible carbohydrates and/or fibers that beneficially affect the host by stimulating the growth and/or the activity of healthy bacteria such as bifidobacteria in one or more regions of the gastrointestinal tract of a subject to which the composition is administered to and/or feeding/stimulating the growth of a microorganism that is contained in the composition that contains the prebiotic, and/or facilitate the production of one or more beneficial metabolites and/or products from the microorganism(s) contained in the composition (see e.g., Gibson G R, Roberfroid M B. Dietary modulation of the human colonic microbiota: introducing the concept of prebiotics. J Nutr. 1995; 125:1401-12; Fan et al., Eur Rev Med Pharmacol Sci.2016. July; 20(15):3262-5; Gupta and Gang. Indian J Med Microbiol. 2009 July-September; 27(3):202-9. doi: 10.4103/0255-0857.53201; Bertelsen et al., Best Pract Res Clin Gastroenterol. 2016 February; 30(1):39-48. doi: 10.1016/j.bpg.2016.01.001. Epub 2016 Jan. 22; Lordan et al., Gut Microbes. 2020; 11(1):1-20. doi: 10.1080/19490976.2019.1613124. Epub 2019 May 22; Salminen et al., Nutrients. 2020 Jun. 30; 12(7):1952. doi: 10.3390/nu12071952; Vandenplas et al., Br J Nutr. 2015 May 14; 113(9):1339-44. doi: 10.1017/S0007114515000823). The prebiotic may be selected from the group consisting of oligosaccharides, optionally containing fructose, galactose, mannose; dietary fibers, in particular soluble fibers, soy fibers; inulin; or mixtures thereof. Preferred prebiotics are fructo-oligosaccharides (FOS), galacto-oligosaccharides (GOS), isomalto-oligosaccharides, xylo-oligosaccharides, oligosaccharides of soy, glycosylsucrose (GS), lactosucrose (LS), lactulose (LA), palatinose-oligosaccharides (PAO), malto-oligosaccharides, pectins and/or hydrolysates thereof

### Vitamins and Minerals

The synthetic formulation may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. One of ordinary skill in the art will, in view of an intended population and the description herein, understand the amount of any given vitamin or mineral appropriate to include.

### Other Components

In some embodiments, the synthetic formulation includes one more suitable emulsifiers and stabilizers such as soy, lecithin, citric acid esters of mono- and diglycerides, and the like. Other suitable stabilizers and emulsifiers will be known to one of ordinary skill in the art.

In some embodiments, the synthetic formulation comprises one or more antioxidants. In some embodiments, the antioxidants are or includes a carotenoid.

In some embodiments, one or more other probiotics in addition to the B. longum microorganisms and compositions thereof of the present invention described elsewhere herein, the compositions can include one or more additional probiotics. The nutritional composition may contain probiotics. The term of art “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally and can be bacteria or yeasts.

The probiotic microorganisms most commonly used are principally bacteria and yeasts of the following genera: Lactobacillus spp., Streptococcus spp., Enterococcus spp., Bifidobacterium spp. and Saccharomyces spp. In some particular embodiments, the probiotic is a probiotic bacterial strain. In some specific embodiments, it is particularly Bifidobacteria and/or Lactobacilli.

Suitable additional probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus johnsonii CNCM 1-1225, Streptococcus salivarius DSM 13084 sold by BLIS Technologies Limited of New Zealand under the designation KI2, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070, Lactobacillus paracasei strain NCC 2461 (see e.g., Demont et al. 2015. J Allergy Clin. Immunol. 137(4): 1264-1267. Bacillus Coagulans Gbi-30 6086, Lactobacillus reuteri Protectis (see e.g., Guiterrez-Castrellon et al. 2014. Pedatrics. March 2014, peds.2013-0652; DOI: https://doi.org/10.1542/peds.2013-0652).

The optional additional probiotics can be included in an amount from about 10³ to 10¹² cfu of probiotic strain, more preferably between 10⁷ and 10¹² cfu such as between 10⁸ and 10¹⁰ cfu of probiotic strain per g of composition on a dry weight basis. In one embodiment the optional additional probiotics are viable. In another embodiment the additional probiotics are non-replicating or inactivated. There may be both viable probiotics and inactivated additional optional probiotics in some other embodiments.

### Composition Formats

### Infant Formulas and Milk Fortifiers

The synthetic formulation can be for example an infant formula, a starter infant formula, a follow-on or follow-up formula, a baby food, an infant cereal composition, a fortifier, such as a human milk fortifier, or a supplement, a growing-up milk, or transitional (or complementary) nutritional composition. The expression “infant formula” means a foodstuff intended for particular nutritional use by infants during the first four to six months of life and satisfying by itself the nutritional requirements of this category of person (Article 1.2 of the European Commission Directive 91/321/EEC of May 14, 1991 on infant formulae and follow-on formulae and/or U.S. Food and Drug Administration Regulations as codified in Title 21 of the U.S. Code of Federal Regulations, particularly at 21 C.F.R. § 107.100—Nutrient Requirements for Infant Formulas). As used herein, “fortifier” refers to liquid, semi-solid, or solid nutritional compositions suitable for mixing with breast milk or infant formula. As used herein, “follow-on formula” or “follow-up formula” means a foodstuff intended for particular nutritional use by infants aged over four months and constituting the principal liquid element in the progressively diversified diet of this category of person. As used herein, “starter formula” refers to an infant foodstuff intended for particular nutritional use by infants during the first four months of life

The term of art, “baby food” means a foodstuff intended for particular nutritional use by infants during the first few years of life, typically about 0.5-5 years of life.

As used herein, the term “infant cereal product” relates to a cereal product that has been designed specifically for infants in order to provide the required nutritional contribution to the infant. Infant cereal products can be grouped in two main categories: complete cereal product which need to be reconstituted in water as they already contain all the necessary nutrients to be delivered with the meal; and standard cereal product which are meant to be reconstituted with milk, infant formula, follow-on formula and/or GUMs.

As used herein, the term “Growing up milk (GUM)” refers to a nutritional composition, which can be given to children from the age of 12 months, and in some instances, after stopping the infant formula. The “growing-up milks” (or GUMs) are given typically from one year onwards. GUMs are generally a milk-based beverage adapted for the specific nutritional needs of young children.

The expression “complementary nutritional composition” or “a nutritional composition for a complementary feeding period”, “transitional nutritional composition” or “a nutritional composition for a transitional feeding period”, as used interchangeably herein, means or refers to a nutritional composition described herein of the present invention, which is designed to be administered to an infant and/or young child before, during, and/or after at the time the transitional feeding period starts. In one embodiment, the transitional nutritional composition is administered during the transitional feeding period. The transitional nutritional composition can be taken/administered enterally, orally, parenterally or intravenously, and it typically includes a lipid or fat source, a protein source and a carbohydrate source. Optionally, the transitional nutritional compositions also comprise vitamins and minerals. Preferably, a transitional nutritional composition is an oral formulation. Non-limiting examples of transitional nutritional compositions are: infant cereal products, follow-on formula, GUMS, or a baby food.

In some particular embodiments, the composition of the invention is an infant formula, a fortifier, or a supplement that may be intended for the first 1, 2, 3, 4, 5, 6, 7, 8, 9, 10, 11, 12, 13, 14, 15, 16, 17, 18, 19, 20, or 24, or any range therein such as 1-6, 6-12, 12-18, 18-24, 2-6, 4-6, 2-12, 4-12, 12-16, or 16-24 months of age. In a preferred embodiment the synthetic formulation of the invention is an infant formula.

In some other embodiments, the synthetic formulation is a fortifier. The fortifier can be a breast milk fortifier (e.g., a human milk fortifier) or a formula fortifier such as an infant formula fortifier or a follow-on/follow-up formula fortifier.

When the synthetic formulation is a supplement, it can be provided in the form of unit doses.

The synthetic formulation can be in solid (e.g., powder), liquid or gelatinous form.

### Supplements

In some embodiments the synthetic formulation is in the form of a supplement. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatin of any origin, vegetable gums, lignin-sulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USDA and/or FDA.

The B. longum microorganisms, compositions, and synthetic formulations can, in some embodiments be formulated as a ready to use formulation. As used herein, the phrase “ready to consume” refers to the formulation being directly ready for use, such as consumption or administration, by a subject (e.g., and infant) without any required further modifications to the formulations. It will be appreciated that while modifications, such as dilution, are not required by a ready to use formulation, such suitable modifications can be applied to a ready to use formulation prior to use.

In some embodiments, the synthetic formulations are ready to use formulations. In some embodiments, the ready to use formulation contains about 0.0001 to about 10 percent by weight or by volume of a B. longum microorganism population or synthetic composition thereof described herein. In some embodiments, the ready to use formulation contains about 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, to/or about 0.099 by weight or volume of a B. longum microorganism population or composition thereof described herein. In some embodiments, the ready to use formulation contains about 0.01, 0.02, 0.03, 0.04, 0.05, 0.06, 0.07, 0.08, 0.09, to/or about 0.1 percent by weight or volume of a B. longum microorganism population or composition thereof described herein.

### Exemplary Compositions

Now provided are some non-limiting exemplary formulations for infant use that can further include one or more B. longum microorganisms and/or genetically modified organisms described herein. One of ordinary skill in the art in view of this disclosure will appreciate expansions of these examples herein that can be applied to other contexts.

For example, a standard infant cereal (to be prepared with milk) according to the present invention to be to be administered to infants at the age of 4-6 months may have an energy density of 220-240 kJ/15 g, 0.8-1.2 g/15 g of a protein source, 0.1-0.3 g of a fat source and 12.3-12.7 g/15.0 g of a carbohydrate source. Such an infant cereal may contain, for example, Rice flour, Maize Maltodextrin, Vitamin C, and Iron. Another example infant cereal to be prepared with water according to the present invention to be administered to infants from the age of 4-6 months may have an energy density of 400-420 kcal/100 g, 10-16 g of a protein source, 7-17 g of a fat source and 50-75 g of a carbohydrate source. Such an infant cereal may contain, for example, Rice flour, Maize Maltodextrin, Vitamin C, and Iron.

Another example, infant cereal according to the present invention to be administered to infants at the age of 6-12 months may have an energy density of 510-525 kcal/100 g, 9-15 g of a protein source, 20-30 g of a fat source and 50-75 g of a carbohydrate source. Such an infant cereal may contain, for example, Wheat flour, Semolina from wheat, Iron, Vitamin C, Niacin, Vitamin B6, Thiamin, and Maize Maltodextrin.

Exemplary infant cereals may be prepared from one or more milled cereals, which may constitute at least 25 weight-% of the final mixture on a dry weight basis.

The infant cereals of the present invention are preferably prepared from a single grain—like rice cereal or wheat cereal—because single grain compositions are less likely to cause an allergic reaction.

Typically, infant cereals are to be mixed with water or milk before consumption. For example, 15 g of an infant cereal of the present invention may be to be mixed with 45 mL (complete infant cereal) of water or 90 ml of milk (standard infant cereal) respectively. METHODS OF USE

The B. longum subspecies microorganisms and modified microorganisms, B. longum subspecies signatures, and compositions thereof can be used for a variety of applications. B. longum subspecies microorganisms and modified microorganisms and compositions thereof can be administered to a subject in need thereof as a preventative or therapeutic. The signatures can be used to analyze a microbiome structure of a microbiome of a subject, which can be useful to provide insight as to the health, physiologic, and/or nutritional state of the subject. The signatures can also be used to identify microbiome structure modulating agents. Agents capable of modifying the microbiome can then be administered to a subject to modify a microbiome as a subject and thus provide a treatment or prevention to the subject.

Transitioning from a Milk-Based Diet to Solid Food

The compositions and formulations according to the present invention can be for use in infants and/or young children. The infants may be born term or are preterm. The compositions and formulations according to the present invention can used in an infant and/or a young child that was born by C-section or that was vaginally delivered.

The compositions and formulations according to the present invention can be for use before, during, and/or after the weaning period (e.g., transitional feeding period). The compositions and formulations according to the present invention can be for use before, during, and/or after the transition from a milk-based diet to a solid food diet.

In some embodiments, a method for, in an infant and/or a young child, promoting/assisting transition from a milk-based diet to solid food and/or promoting gut microbiota adapted to metabolize both milk derived carbohydrates and fibers, or any derivative thereof includes administering a synthetic formulation described in greater detail elsewhere herein to the infant.

In some embodiments, a method for, in an infant and/or a young child, promoting/assisting transition from a milk based diet to solid food and/or promoting gut microbiota adapted to metabolize both milk derived carbohydrates and fibers or a derivative thereof includes administering, to the infant and/or young child, one or more Bifidobacterium longum (B. longum) transitional clade microorganisms, which have at least 98.6% Average Nucleotide Identify (ANI) with at least one strain selected in the group of CNCM I-5683, CNCM I-5684, CNCM I-5685, CNCM I-5686 and CNCM I-5687. In some embodiments of the method, the one or more B. longum transitional clade microorganisms have about 98.6%-100% Average Nucleotide Identity (ANI) with at least one strain selected from the group of: NCC5000, NCC5001, NCC5002, NCC5003 and NCC5004. In some embodiments of the method, the one or more B. longum transitional clade microorganisms have about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one strain selected from the group of: CNCM I-5683, CNCM I-5684, CNCM I-5685, CNCM I-5686 and CNCM I-5687. In some embodiments of the method, the one or more B. longum transitional clade microorganisms have at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one strain selected from the group of: CNCM I-5683, CNCM I-5684, CNCM I-5685, CNCM I-5686 and CNCM I-5687. In some embodiments of the method, the one or more B. longum transitional clade microorganisms have at least 98.6% ANI with at least one strain selected from the group of: CNCM I-5683, CNCM I-5684, CNCM I-5685, CNCM I-5686 and CNCM I-5687.

In some embodiments of the method, the one or more B. longum transitional clade microorganisms have about 98.6%-100% ANI with at least one genome sequence selected from the group of SEQ ID NOs: 1-4010. In some embodiments of the method, the one or more B. longum transitional clade microorganisms have about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one genome sequence selected from the group of SEQ ID NOs: 1-4010. In some embodiments of the method, the one or more B. longum transitional clade microorganisms have at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one genome sequence selected from the group of SEQ ID NOs: 1-4010.

In some embodiments of the method, the one or more B. longum transitional clade microorganisms are characterized by the presence of all genes from Table 1. In some embodiments, the one or mores B. longum transitional clade microorganism are characterized by the presence of one or more genes from Table 1. In some embodiments of the method, the one or more B. longum transitional clade microorganisms are characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1. In some embodiments of the method, the one or more B. longum transitional clade microorganisms are characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1.

In some embodiments of the method, the one or more B. longum transitional clade microorganisms are characterized by the presence of all genes from Table 2. In some embodiments of the method, the one or more B. longum transitional clade microorganisms are characterized by the presence of one or more genes from Table 2. In some embodiments of the method, the one or more B. longum transitional clade microorganisms are characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2. In some embodiments of the method, the one or more B. longum transitional clade microorganisms are characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2.

In some embodiments of the method, the one or more B. longum microorganisms is not of the subspecies B. longum subspecies longum or B. longum subspecies infantis.

In some embodiments of the method, the one or more B. longum microorganisms are isolated from a human.

In some embodiments, a method for, in an infant or a young child, promoting/assisting transition from a milk-based diet to solid food and/or promoting gut microbiota adapted to metabolize both milk derived carbohydrates and fibers, or a derivative thereof includes administering, to the infant, one or more genetically modified microorganisms as described in greater detail elsewhere herein. In some embodiments of the method, the genetically modified microorganism is not of a B. longum transitional clade and is modified to have a B. longum transitional clade signature. In some embodiments of the method, the genetically modified microorganism is not of a B. longum transitional clade and is modified to have modified expression of one or more genes from Tables 1 and/or 2. In some embodiments of the method, the genetically modified microorganism is not of a B. longum transitional clade and is modified to have modified expression of all genes from Tables 1 and/or 2.

The compositions and formulations according to the present invention can be used for treatment and/or prevention purposes.

In some embodiments, the compositions and formulations according to the present invention are administered immediately after birth of an infant. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4 or 6 months of birth of said infant.

In some other embodiments, the compositions and formulations according to the present inventions is given few days (e.g., 1, 2, 3, 5, 10, 15, 20, or more), or few weeks (e.g., 1, 2, 3, 4, 5, 6, 7, 8, 9, 10, or more), or few months (e.g., 1, 2, 3, 4, 5, 6, 7, 8, 9, 10, 11, 12, 13, 14, 15, 16, 17, 18, 19, 20, 21, 22, 23, 24, or more) after birth.

In one embodiment, the compositions and formulas of the invention is given to the infant and/or young child as a supplementary composition to the mother's milk. As used herein, “mother's milk” should be understood as the breast milk or colostrum of the mother. In some embodiments the infant or young child receives the mother's milk during at least the first 2 weeks, first 1, 2, 4, or 6 months. In one embodiment the nutritional composition of the invention is given to the infant or young child after such period of mother's nutrition is given together with such period of mother's milk nutrition. In some embodiments, the composition is given to the infant or young child as the sole or primary nutritional composition during at least one period of time, e.g., after the 1st, 2nd or 4^{th} month of life, during at least 1, 2, 4, 5, 6, 7, 8, 9, 10, 11, 12, 13, 14, 15, 16, 17, 18, 19, 20, 21, 22, 23, or at least 24 months after birth. In some embodiments, the composition is given to the infant or young child as the supplementary or secondary nutritional composition (e.g., secondary to a mother's milk or solid food) during at least one period of time, e.g., after the 1st, 2nd or 4^{th} month of life, during at least 1, 2, 4, 5, 6, 7, 8, 9, 10, 11, 12, 13, 14, 15, 16, 17, 18, 19, 20, 21, 22, 23, or at least 24 months after birth.

In some embodiments, the compositions and formulations described herein is formulated as a complete nutritional composition (fulfilling all or most of the nutritional needs of the subject). In another embodiment the nutrition composition is a supplement, or a fortifier intended for example to supplement human milk or to supplement an infant formula or a follow-on formula.

In some embodiments, the transitional composition for use according to the invention is administered to an infant and/or young child of an age of between about 4 to about 12, 24, 36, 48 60 months and administration should last for at least 1-7 days, 1 week, 2 weeks, 3 weeks, 4 weeks, 1 month, 2 months, 3 months, or 4 months or more, for example 5, 6, 9, or 12 months or more.

In the above-mentioned period, administration may be for example intermittent, or for example on average once daily over said the period, or for example once every other day over said period, or for example at least once daily during said period.

In some embodiments, the administration of the composition is for a shorter duration of time falling within the time period mentioned above, for example on average once daily for a duration of at least six weeks in said time period, such as for example on average once daily for 3, 6, 8, 9, or 12 months during said time period.

In some embodiments, the administration may be for example on average once every other day for a period of at least six weeks in the period from 4 months to 60 months, such as for example on average once every other day for a period of 3, 6, 8, 9, or 12 months during said time period.

In some embodiments, the transitional nutritional composition is administered once daily. In some embodiments, the once daily administration occurs for 1, 2, 3, 4, 5, 6, 7, 8, 9, 10, 11, 12, 13, 14, 15, 16, 17, or 18 or more days, weeks, or months.

### Identifying Microbiome Structure and Microbiome Modulating Agents

The B. longum transitional clade and signatures can be used in an assay to analyze a microbiome structure to determine the abundance (relative or absolute) of B. longum transitional clade microorganisms in the microbiome, such as in a gastrointestinal microbiome. Further, the B. longum transitional clade genes and signatures can be used in an assay to identify one or more candidate or test agents capable of modulating a microbiome, such as a gastrointestinal microbiome, such that the microbiome has increased and/or decreased abundance of one or more B. longum transitional clade microorganisms. As used herein, “microbiome structure” refers to the profile of different microorganisms or microorganism diversity within a microbiome.

### Identifying Microbiome Structure

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more Bifidobacterium longum (B. longum) microorganisms characterized by at least 98.6% Average Nucleotide identity (ANI) with at least one strain selected in the group consisting of: CNCM I-5683, CNCM I-5684, CNCM I-5685, CNCM I-5686 and CNCM I-5687. Suitable methods or techniques for measuring the abundance (relative or absolute) include any gene and/or expression analysis techniques, such as any of those discussed elsewhere herein in relation to genes and signatures and others that will be appreciated by those of ordinary skill in the art.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more Bifidobacterium longum (B. longum) microorganisms characterized by having about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one strain selected from the group of: NCC5000, NCC5001, NCC5002, NCC5003 and NCC5004.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more Bifidobacterium longum (B. longum) microorganisms characterized by having at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one strain selected from the group of: CNCM I-5683, CNCM I-5684, CNCM I-5685, CNCM I-5686 and CNCM I-5687.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more Bifidobacterium longum (B. longum) microorganisms characterized by having about 98.6%-100% ANI with at least one genome sequence selected from the group of SEQ ID NOs: 1-4010.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more Bifidobacterium longum (B. longum) microorganisms characterized by having about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one genome sequence selected from the group of SEQ ID NOs: 1-4010.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more Bifidobacterium longum (B. longum) microorganisms characterized by having at least 98.6%, has at least 98.7%, has at least 98.8%, has at least 98.9%, has at least 99%, has at least 99.1%, has at least 99.2%, has at least 99.3%, has at least 99.4%, has at least 99.5%, has at least 99.6%, has at least 99.7%, has at least 99.8%, has at least 99.9%, ANI with at least one genome sequence selected from the group of SEQ ID NOs: 1-4010.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of all genes from Table 1.

In some embodiments, a method of determining the abundance of one or more B. longum transitional clade microorganisms in a sample from a subject can include determining, using one or more suitable methods or techniques, a microbiome structure of a gastrointestinal tract of the subject by at least determining the relative abundance of one or more B. longum transitional clade microorganisms that are characterized by the presence of one or more genes from Table 1.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of all genes from Table 2.

In some embodiments, a method of determining the abundance of one or more B. longum transitional clade microorganisms in a sample from a subject can include determining, using one or more suitable methods or techniques, a microbiome structure of a gastrointestinal tract of the subject by at least determining the relative abundance of one or more B. longum transitional clade microorganisms that are characterized by the presence of one or more genes from Table 2.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30 genes from Table 2.

In certain example embodiments of a method, the one or more B. longum microorganisms is not of the subspecies B. longum subspecies longum or B. longum subspecies infantis.

In certain example embodiments of a method, the one or more B. longum microorganisms are isolated from a human.

In some embodiments of a method, the sample is a biological sample. In some embodiments, the sample is a biological sample containing contents from one or more regions from a gastrointestinal tract (e.g., mouth, esophagus, stomach, small intestine (duodenum, jejunum, and/or ileum), appendix (when present), large intestine, cecum (when present), or any combination thereof. In some embodiments, the sample is a stool sample. In some embodiments of the method, the subject is a mammal. In some embodiments of a method, the subject is a non-human mammal. In some embodiments of the method, the subject is a human. In some embodiments of the method, the subject is an infant and/or young child.

### Identifying Microbiome Modulating Agents

The B. longum transitional clade signatures can be used in assays to identify microbiome modulating agents. Generally, identifying an agent capable of modulating a microbiome structure includes a) applying a candidate agent to a cell or cell population from a microbiome of a subject or a sample including the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., a B. longum transitional clade gene signature). In certain embodiments, steps can include administering candidate modulating agents to cells or sample containing cells, detecting identified cell (sub)populations for changes in signatures, or identifying relative changes in cell (sub) populations which may comprise detecting relative abundance of particular gene signatures, particular a B. longum transitional clade gene signature.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

Test or candidate agents include chemical and/or biologic agents as well as environmental factors or stressors. Environmental factors or stressors include, without limitation heat shock, osmolarity, hypoxia, cold, oxidative stress, radiation, starvation. Chemical and/or biologic agents include, without limitation, small molecule organic or inorganic compounds, proteins, peptides, amino acids, nucleic acids, polynucleotides, and combinations thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

In some embodiments, a method of determining the abundance of one or more B. longum transitional clade microorganisms in a sample from a subject includes determining, using one or more suitable methods or techniques, a microbiome structure of a gastrointestinal tract of the subject by at least determining the relative abundance of one or more B. longum transitional clade microorganisms that are characterized by the presence of a B. longum transitional signature. B. longum transitional clade signatures are described in greater detail elsewhere herein.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by at least 98.6% Average Nucleotide identity (ANI) with at least one strain selected in the group of: CNCM I-5683, CNCM I-5684, CNCM I-5685, CNCM I-5686 and CNCM I-5687, wherein the gastrointestinal microbiome modulating agent is effective to increase or decrease the relative abundance of one or more of the one or more B longum microorganism characterized by the presence of the B longum transitional clade genes. Suitable methods or techniques for measuring the abundance (relative or absolute) include any gene and/or expression analysis techniques, such as any of those discussed elsewhere herein in relation to genes and signatures and others that will be appreciated by those of ordinary skill in the art.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having at least 98.6%, by having at least 98.7%, by having at least 98.8%, by having at least 98.9%, by having at least 99%, by having at least 99.1%, by having at least 99.2%, by having at least 99.3%, by having at least 99.4%, by having at least 99.5%, by having at least 99.6%, by having at least 99.7%, by having at least 99.8%, by having at least 99.9%, ANI with at least one strain selected from the group of: CNCM I-5683, CNCM I-5684, CNCM I-5685, CNCM I-5686 and CNCM I-5687, wherein the gastrointestinal microbiome modulating agent is effective to increase or decrease the relative abundance of one or more of the one or more B longum microorganism characterized by the presence of the B longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having about 98.6%-100% ANI with at least one genome sequence selected from the group of SEQ ID NOs: 1-4010, wherein the gastrointestinal microbiome modulating agent is effective to increase or decrease the relative abundance of one or more of the one or more B longum microorganism characterized by the presence of the B longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having about 98.6%, 98.7%, 98.8%, 98.9%, 99%, 99.1%, 99.2%, 99.3%, 99.4%, 99.5%, 99.6%, 99.7%, 99.8%, 99.9%, or 100% ANI with at least one genome sequence selected from the group of SEQ ID NOs: 1-4010, wherein the gastrointestinal microbiome modulating agent is effective to increase or decrease the relative abundance of one or more of the one or more B longum microorganism characterized by the presence of the B longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having at least 98.6%, by having at least 98.7%, by having at least 98.8%, by having at least 98.9%, by having at least 99%, by having at least 99.1%, by having at least 99.2%, by having at least 99.3%, by having at least 99.4%, by having at least 99.5%, by having at least 99.6%, by having at least 99.7%, by having at least 99.8%, by having at least 99.9%, ANI with at least one genome sequence selected from the group of SEQ ID NOs: 1-4010, wherein the gastrointestinal microbiome modulating agent is effective to increase or decrease the relative abundance of one or more of the one or more B longum microorganism characterized by the presence of the B longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of all genes from Table 1, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of one or more genes from Table 1, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 genes from Table 1, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of all genes from Table 2, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of one or more genes from Table 2, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more Bifidobacterium longum (B. longum) microorganisms characterized by having one or more B. longum transitional clade microorganisms that are characterized by the presence of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2, wherein the gastrointestinal microbiome modulating agent is effective to increase or decrease the relative abundance of one or more of the one or more B longum microorganism characterized by the presence of the B. longum transitional clade genes.

In certain example embodiments, a method of identifying a gastrointestinal microbiome modulating agent includes administering an amount of a test agent to a subject or to a sample therefrom; and determining a microbiome structure of a gastrointestinal tract of the subject by at least determining the relative abundance of one or more of the one or more Bifidobacterium longum (B. longum) microorganisms characterized by having one or more B. longum transitional clade microorganisms that are characterized by the presence of 1 to 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes from Table 2, wherein the gastrointestinal microbiome modulating agent is effective to increase or decrease the relative abundance of one or more of the one or more B longum microorganism characterized by the presence of the B. longum transitional clade genes.

In certain embodiments of a method, the test agent is provided in a formulation.

Suitable methods or techniques for measuring the abundance (relative or absolute) of a microorganism include any gene and/or expression analysis techniques, such as any of those discussed elsewhere herein in relation to genes and signatures and others that will be appreciated by those of ordinary skill in the art. Exemplary detection methods include immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

In some embodiments a metagenome analysis can be performed as described elsewhere herein to analyze the microbiome structure and changes therein in response to a candidate agent within the methods described herein.

In certain example embodiments of a method, the one or more B. longum microorganisms is not of the subspecies B. longum subspecies longum or B. longum subspecies infantis.

In certain example embodiments of a method, the one or more B. longum microorganisms are isolated from a human.

In some embodiments of a method, the sample is a biological sample. In some embodiments, the sample is a biological sample containing contents from one or more regions from a gastrointestinal tract (e.g., mouth, esophagus, stomach, small intestine (duodenum, jejunum, and/or ileum), appendix (when present), large intestine, cecum (when present), or any combination thereof. In some embodiments, the sample is a stool sample. In some embodiments of the method, the subject is a mammal. In some embodiments of a method, the subject is a non-human mammal. In some embodiments of the method, the subject is a human. In some embodiments of the method, the subject is an infant or young child.

Test agents identified as microbiome modulating agents as previously described can be administered to a subject in need thereof to modify the structure of a microbiome so as to have increased or decreased amounts of a B. longum transitional clade microorganism. In some embodiments, one or more modulating agents are included in a formulation, such as a synthetic composition described herein, or other composition formulated for administration to a subject in need thereof.

Further embodiments are illustrated in the following Examples which are given for illustrative purposes only and are not intended to limit the scope of the invention.

### EXAMPLES

### Example 1—Initial Metagenome Analysis of Infant Stool Samples Reveals a B. longum Transitional Clade

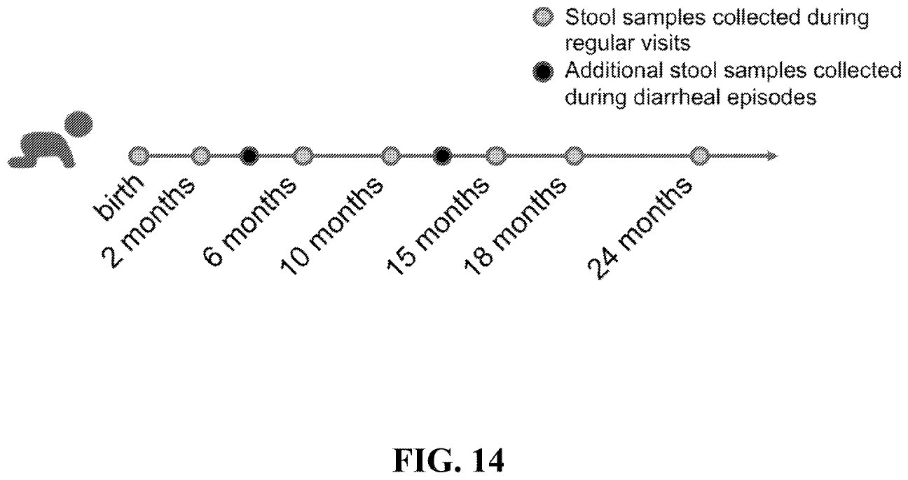

This example discusses a metagenome analysis of the gut microbiome in infants in Dhaka Bangladesh (as determined by evaluating stool samples) over a time period from birth to about 2 years in both health and possible disease states (as indicated by a diarrhea event) (see e.g., FIG. 14). Initially, 1, 377 samples were sequenced for metagenome analysis. On average about 26.4 M sequencing reads were obtained per sample, with an interquartile range of 15.4M-34.8M reads and a standard deviation of 16M reads. 66 samples had less than 5M sequencing reads and were filtered out of this analysis and were resequenced. Of the filtered samples, most were either DIA samples (those obtained during a diarrhea event) or early samples but were not samples collected at birth. Specifically, there were 19 DIA samples filtered, 15 2-month old samples filtered, 10 6-month old samples filtered, 9 10-month old samples filtered, 7 15-month old samples filtered, 3 24-month old samples filtered, 2 18-month old samples filtered, and 1 birth sample filtered.

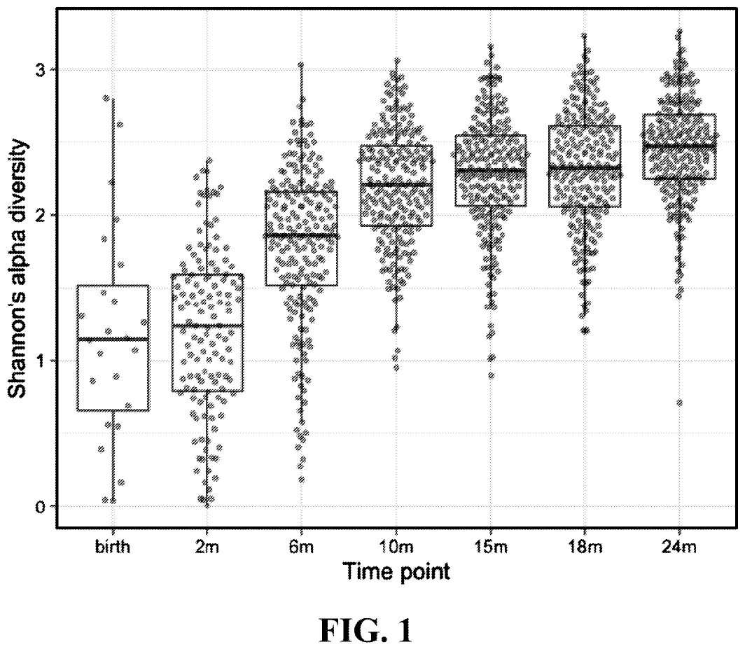

B. longum subspecies infantis was quantified in the samples on hand. Initially it was sought to compare HMO gene cluster genes (those used to detect B. longum subspecies infantis in the DIABIVIMUNE and TEDDY data sets) to core genes of the metagenomic set to determine relative abundance of wrt. B. longum. (HUMAnN2 B. longum-stratified CPM). Then core genes (HUMAnN2 CPM) with the greatest Pearson correlation with B. longum relative abundance (MetaPhAn) was identified. Results of these analyses are shown in FIGS. 1-5.

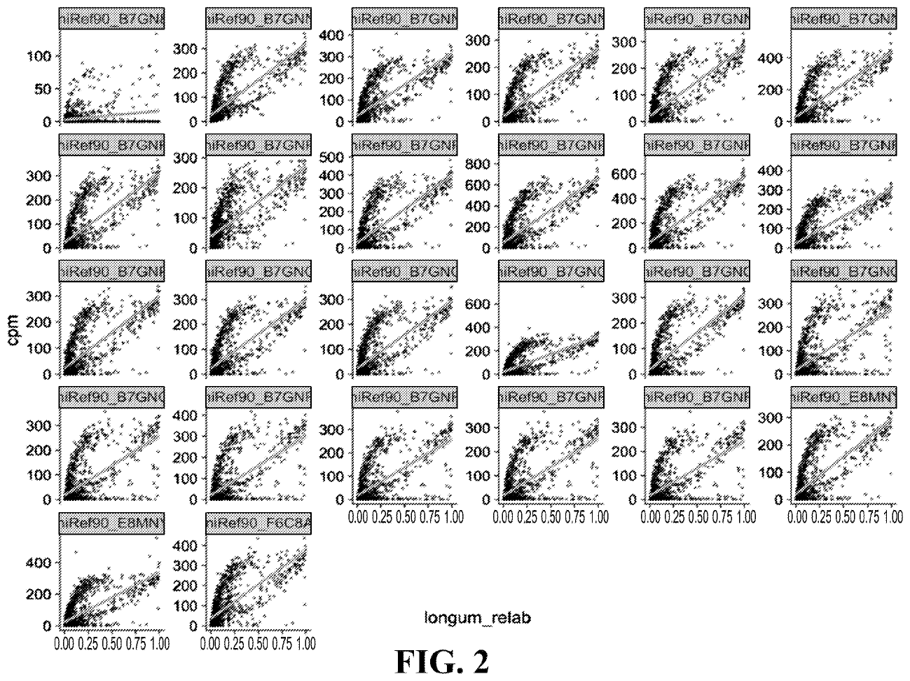

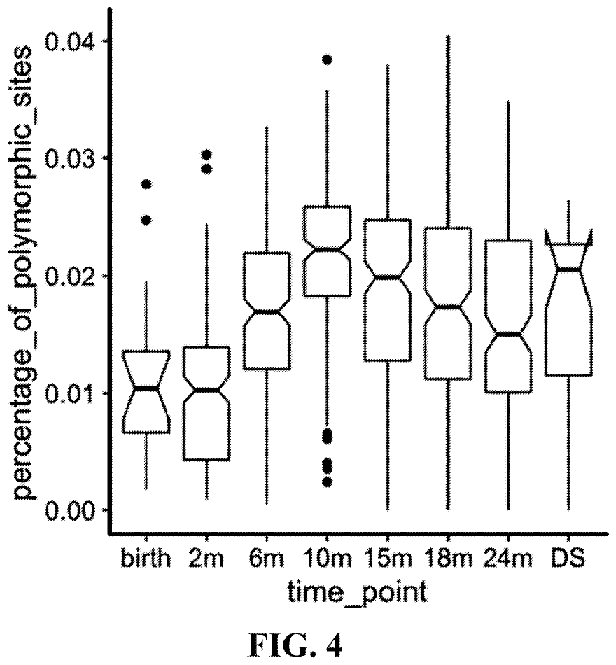

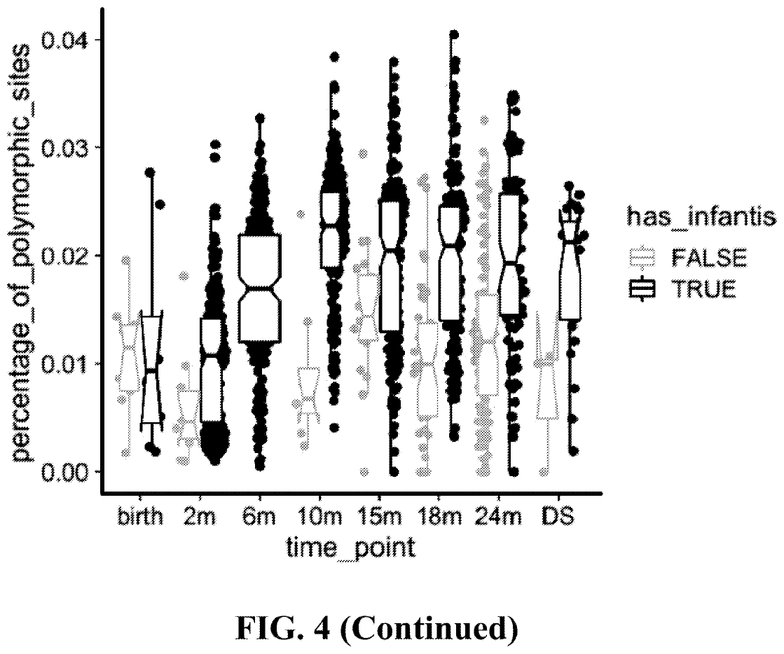

Overall, microbial complexity increased with age (see e.g., FIG. 1). FIG. 2 shows a comparison of HMO cluster genes (used to detect B. longum subspecies infantis in DIABIMMUNE and TEDDY) abundance to core genes to determine the relative abundance wrt. B. longum (HUMAnN2 B. longum-stratified CPM). FIG. 3 shows a discrepancy between B. longum relative abundance (MetaPhlAn) and median core genome coverage (StrainPhlAn). FIGS. 4-5 show that there are more polymorphisms in B. longum during the transition from breast feeding to solid foods.

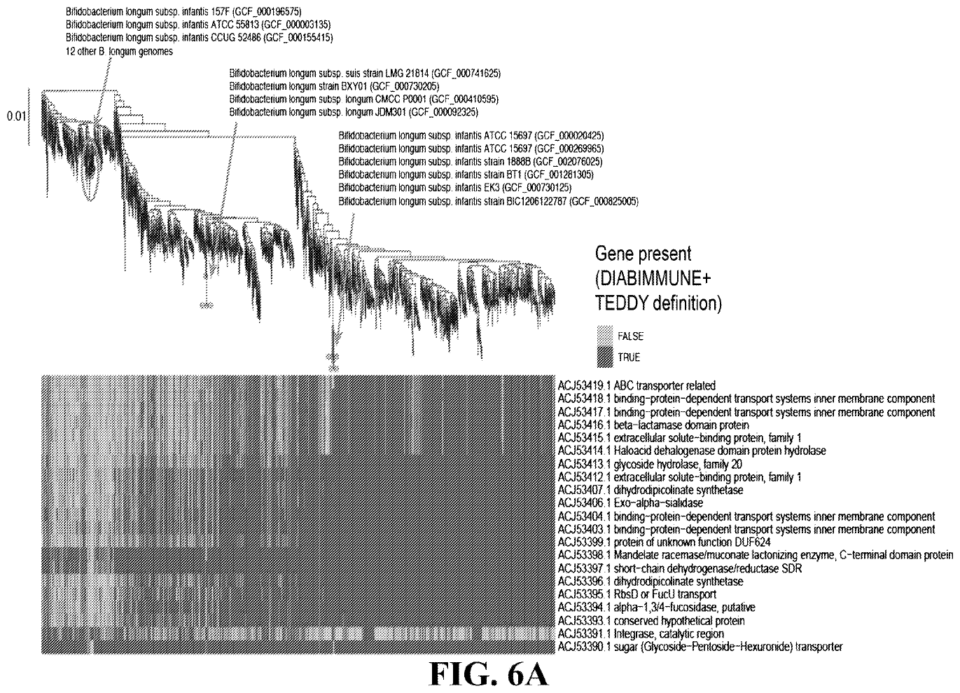

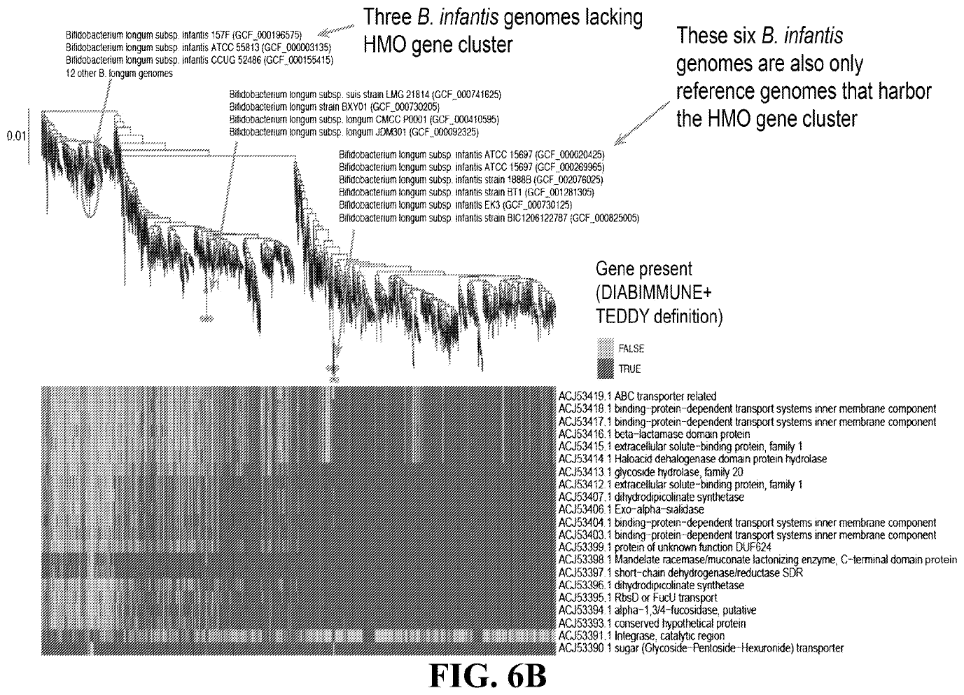

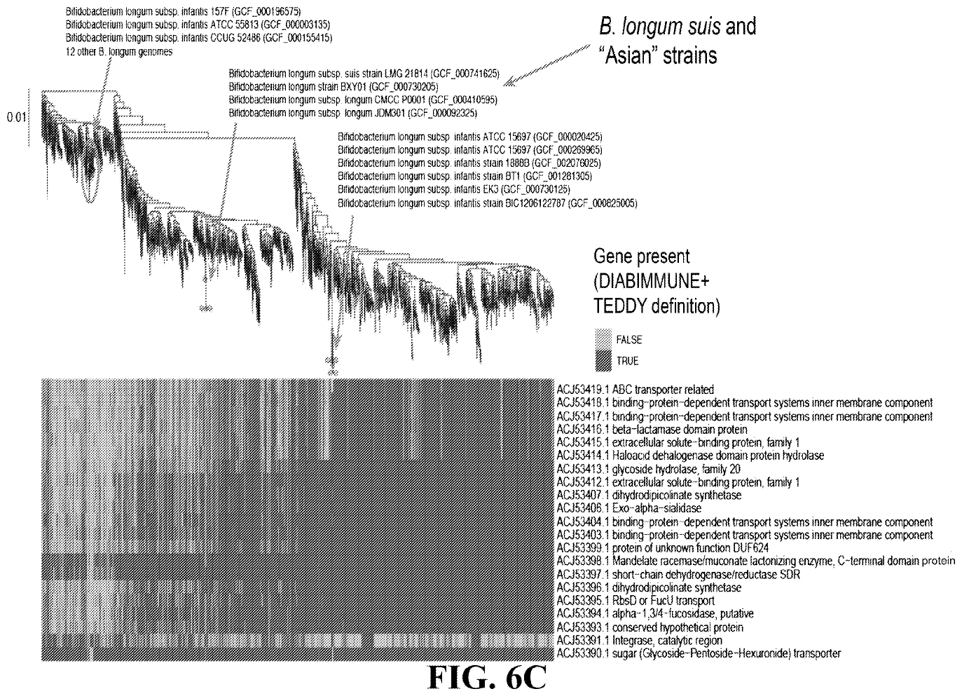

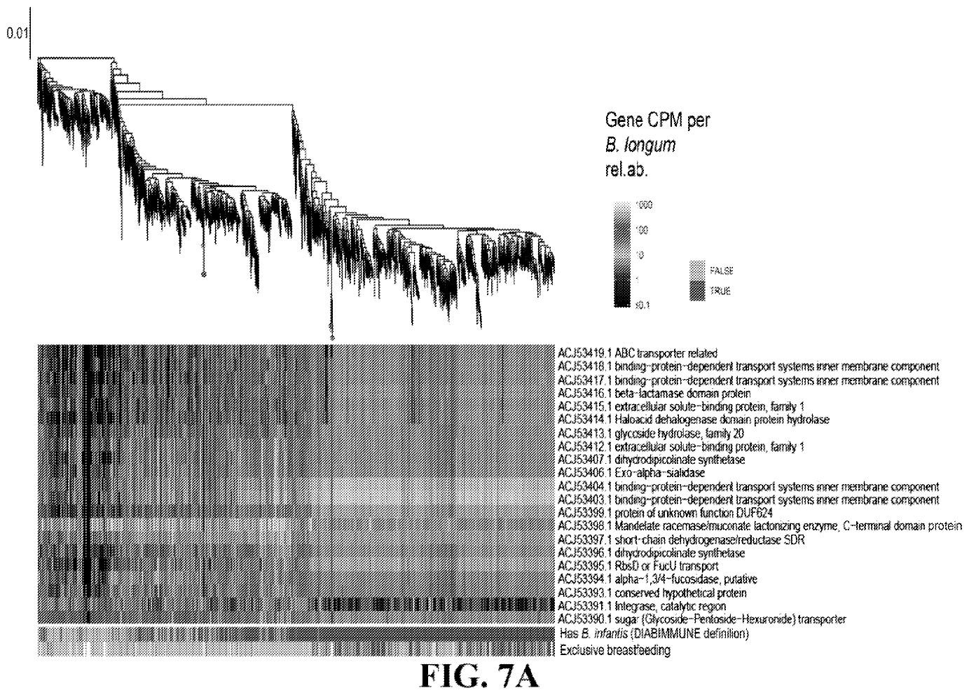

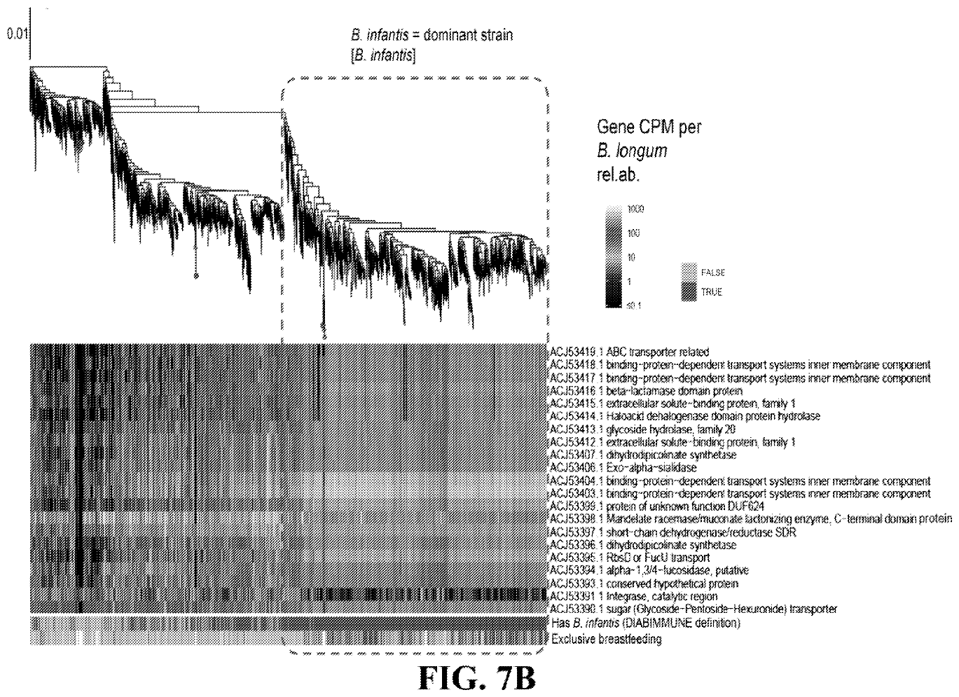

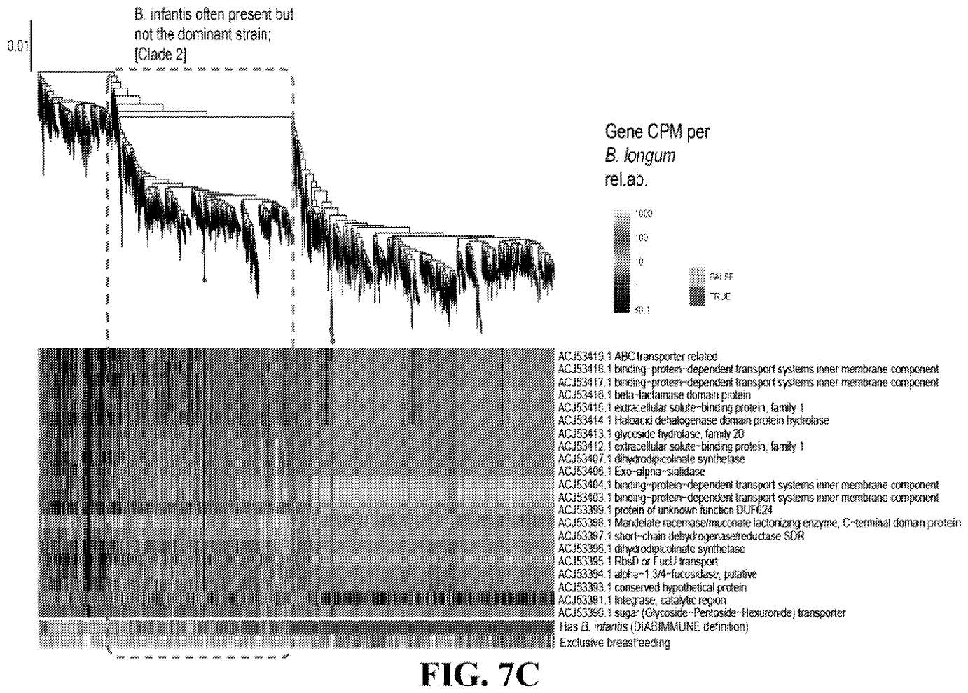

From the analysis of B. longum subspecies infantis, seven new B. longum genomes were included in the strain phylogeny analysis. These were B. longum subspecies infantis strain BIC 1206122787, B. longum subspecies infantis strain BT1, B. longum subspecies infantis EK3, B. longum subspecies infantis strain 1888B, B. longum subspecies suis strain LMG 21814, B. longum subspecies longum (Asian) strain CMCC P0001 and B. longum subspecies longum (Asian) strain BXY01. The strain analysis results and phylogenies are shown in FIGS. 6A-6C and 7A-7D.

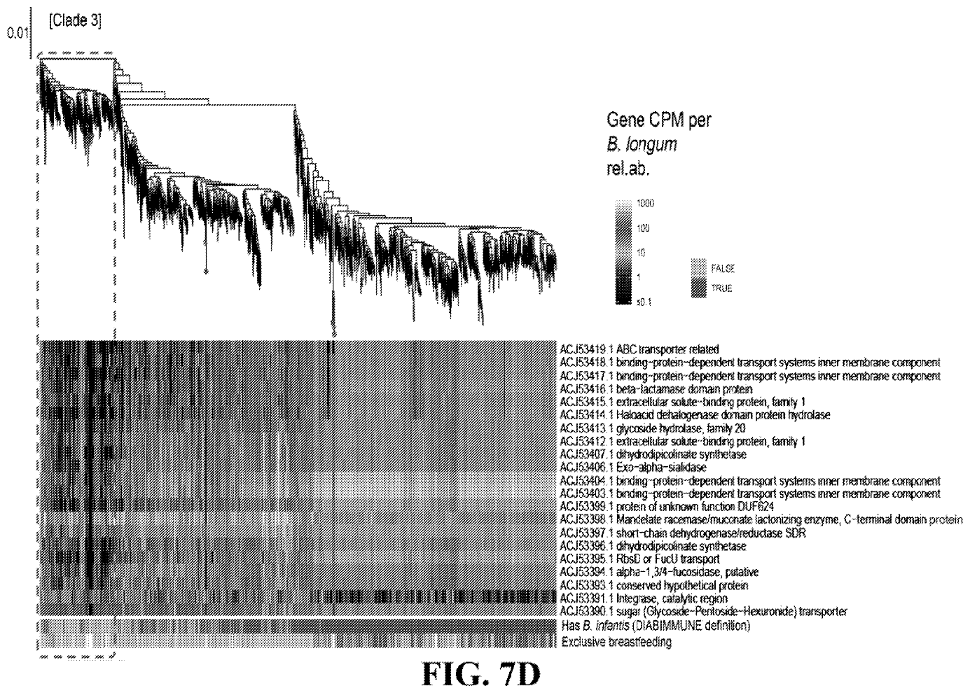

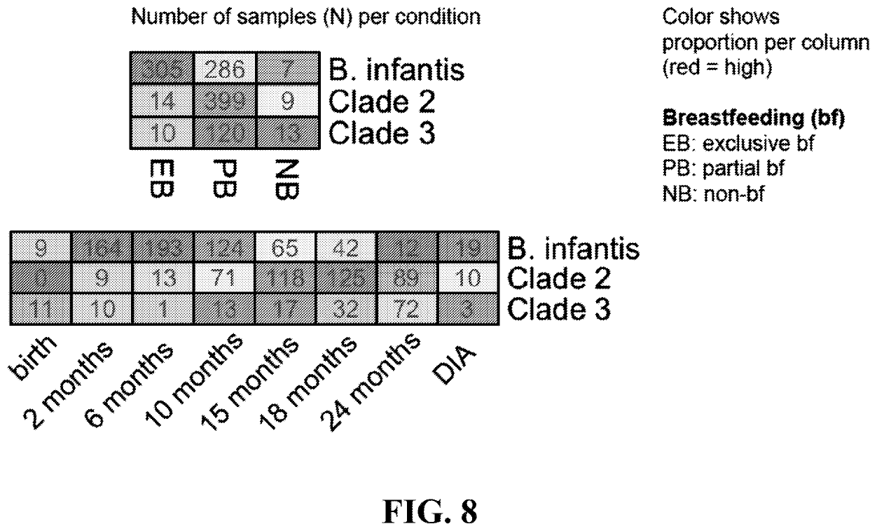

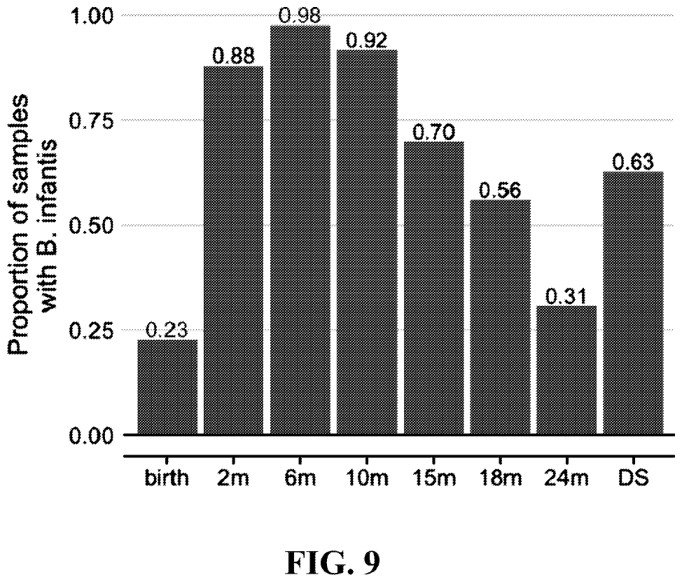

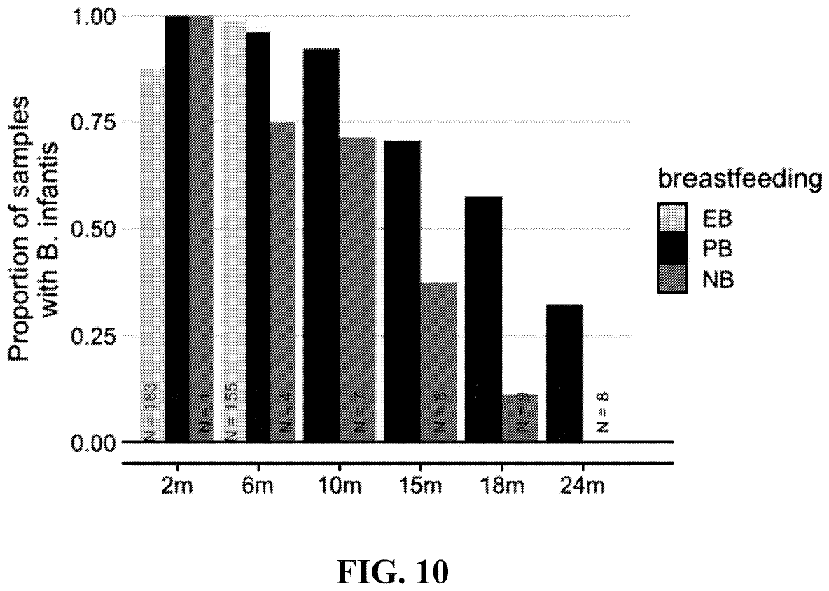

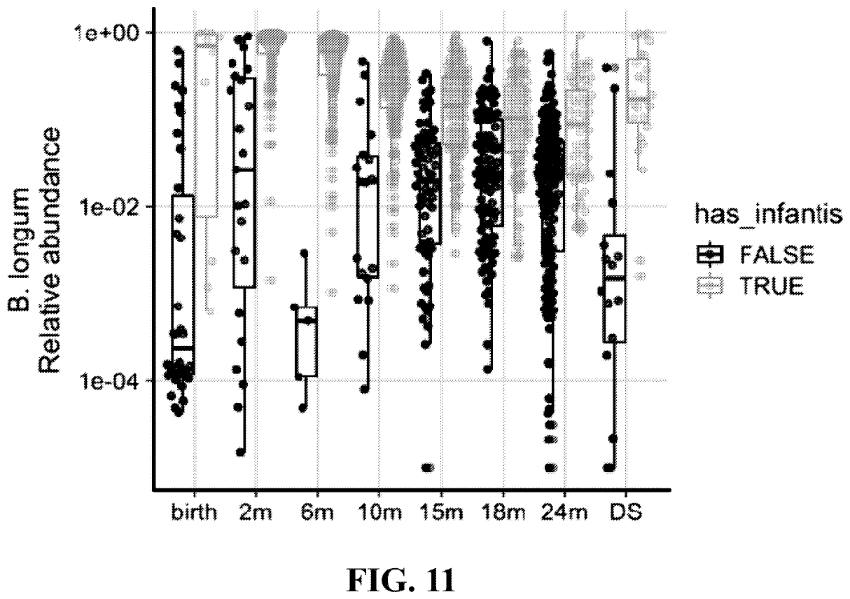

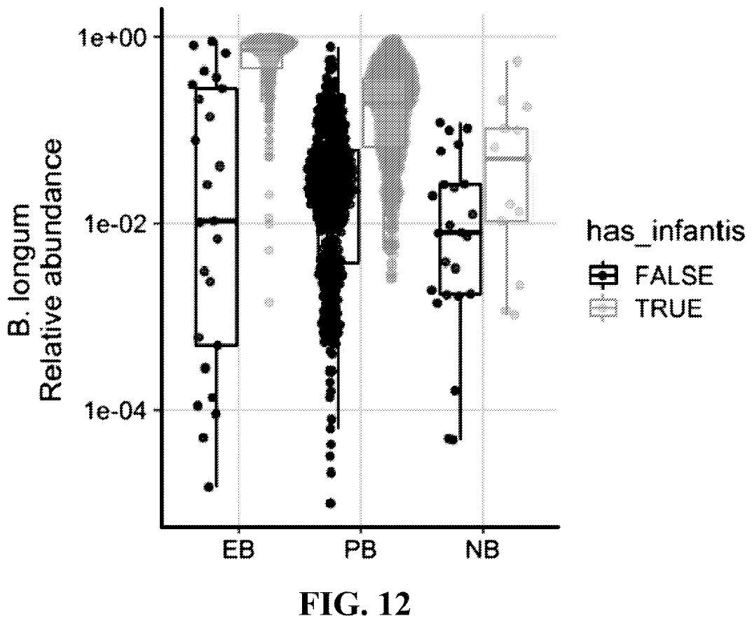

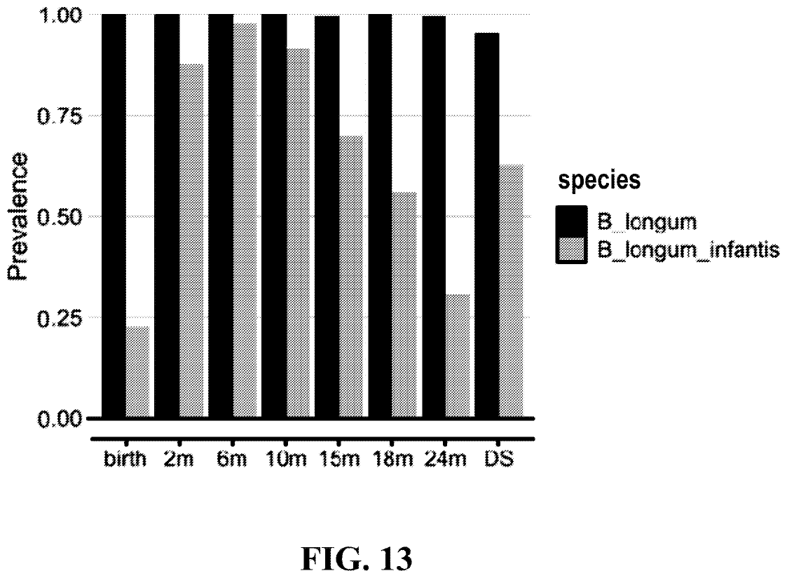

Further analysis revealed a longitudinal B. longum subspecies infantis to Clade 2 (see e.g., FIG. 7C) to Clade 3 transition (see e.g., FIG. 7D) with the introduction of solid foods and weaning (FIG. 8). B. longum subspecies infantis is present in most samples from the Dhaka babies in the cohort. (FIG. 9). 99.6% percent of babies (all but one) had B. longum subspecies infantis in at least one stool sample. Samples had 14 or more B. longum subspecies infantis HMO cluster genes. B. longum subspecies infantis was less common in non-breast-fed babies (FIG. 10) and was more abundant than other B. longum subspecies longum strains (FIG. 11). Further, abundance of B. longum subspecies infantis and other B. longum subspecies longum strains differed during breastfeeding (FIG. 12). It was observed that B. longum subspecies longum was ubiquitous with longitudinal strain shifts, including a change in B. longum subspecies infantis (FIG. 13).

### Example 2—MAG Assembly and Analyses

Further metagenomic analysis was performed. 222 babies were followed in Dhaka, Bangladesh. Metagenomic sequencing and analysis was performed on 1,353 stool samples. The MetaBAT2 wdl workflow was used for binning MAGs, which included MegaHIT assembly, MetaBAT2 binning (using dynamic parameter optimization), checkM to obtain MAG quality (completeness, contamination, etc.) and GTDB-Tk to assign taxonomy. FIG. 14 shows the sampling timeline.

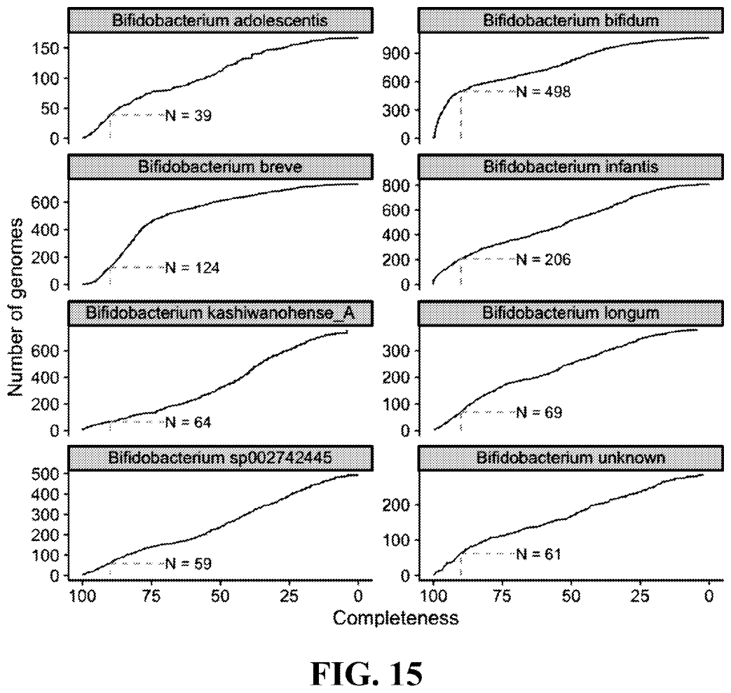

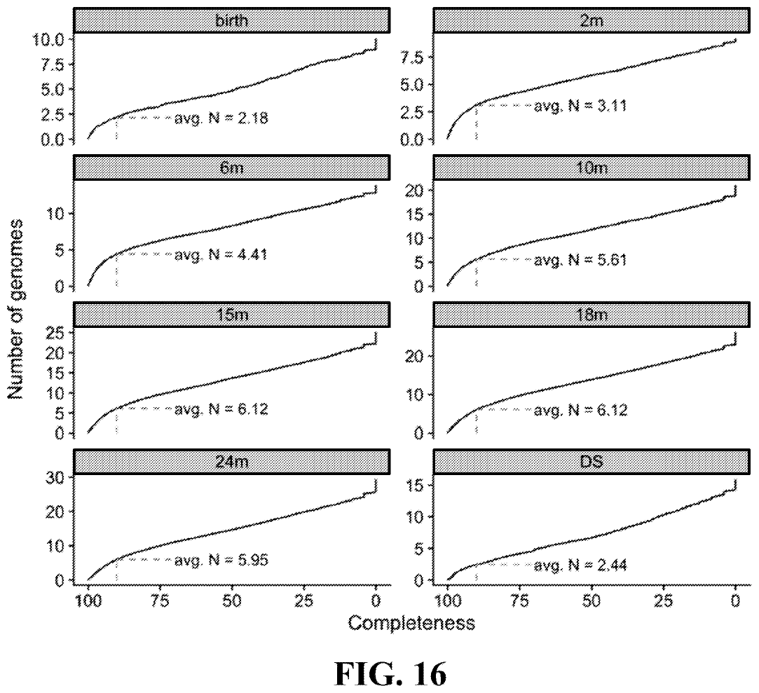

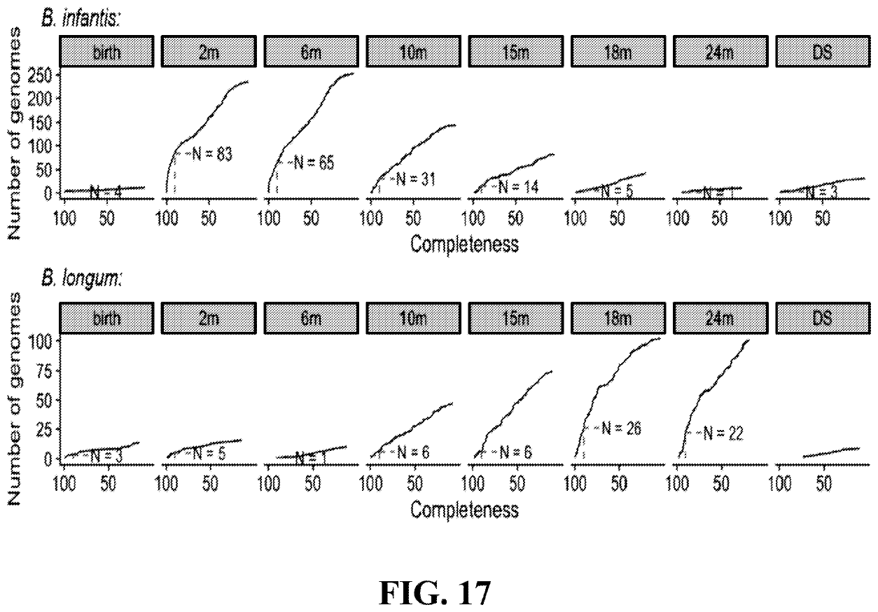

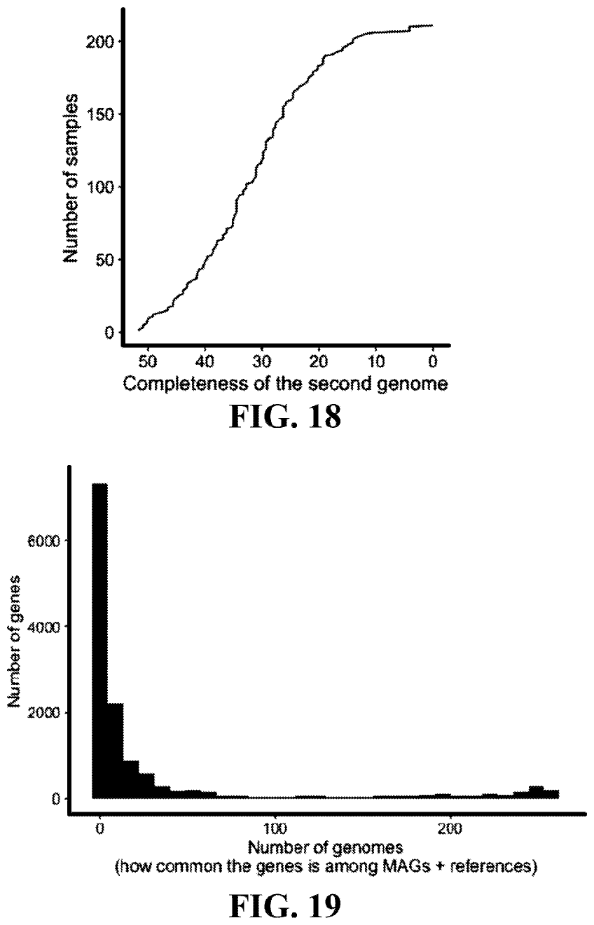

FIG. 15 shows the number of assembled Bifidobacterium genomes per species as a function of genome completeness. The number of genomes with completeness >90 is shown for each species. Only species with more than 50 genomes are shown. FIG. 16 shows the average number of high quality (HQ) genomes per sample and timepoint. FIG. 17 show the number of B. longum subspecies infantis and B. longum subspecies longum genomes (completeness >90) over time. FIG. 18 shows that there were no multiple HQ genomes (completeness >90) B. longum genomes. N=8 samples with second genome with completeness >50.

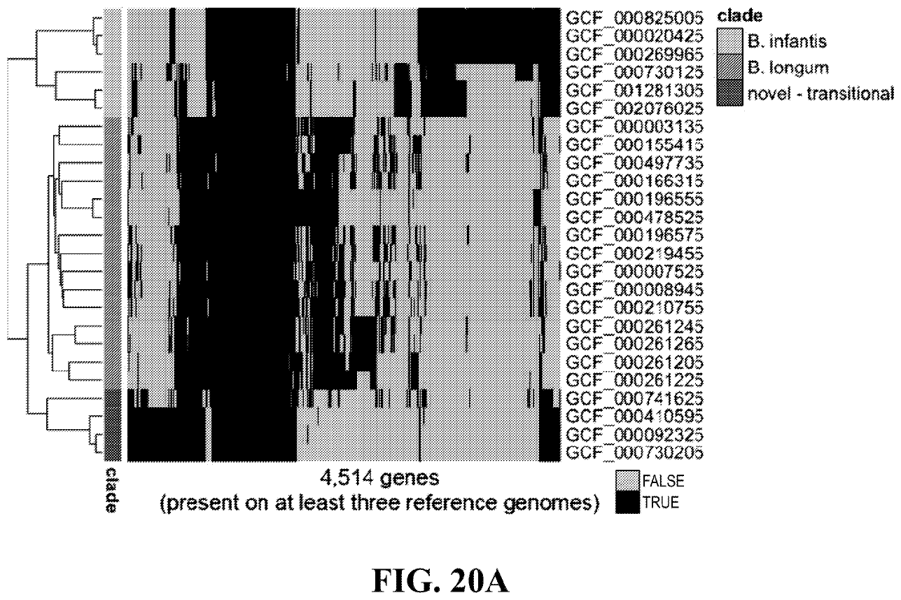

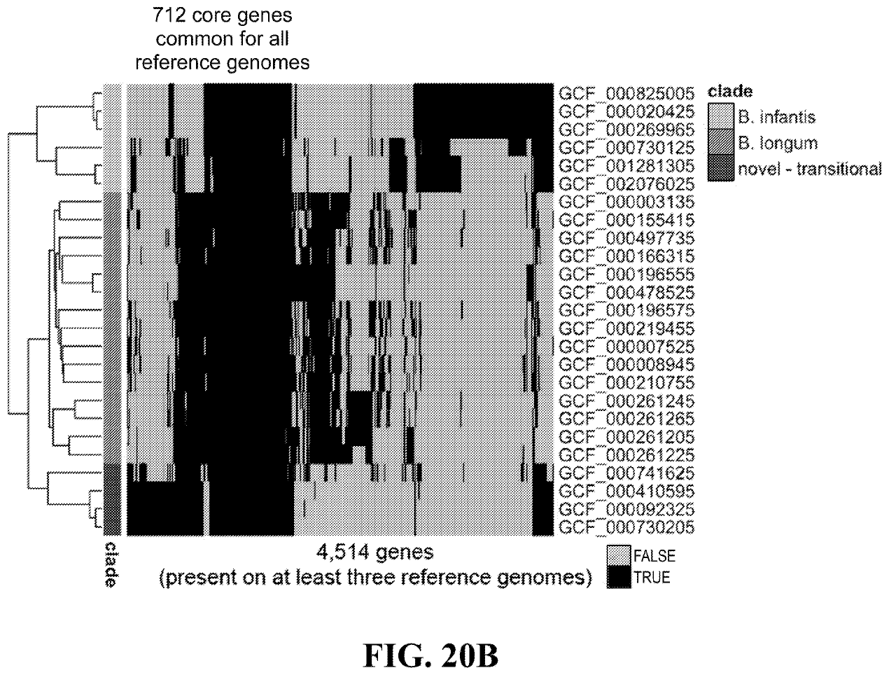

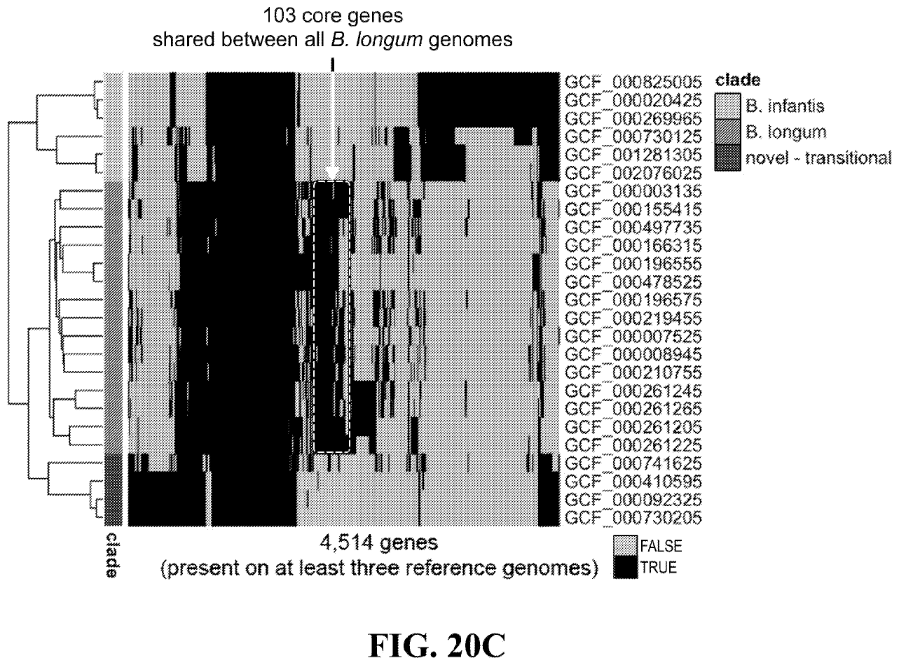

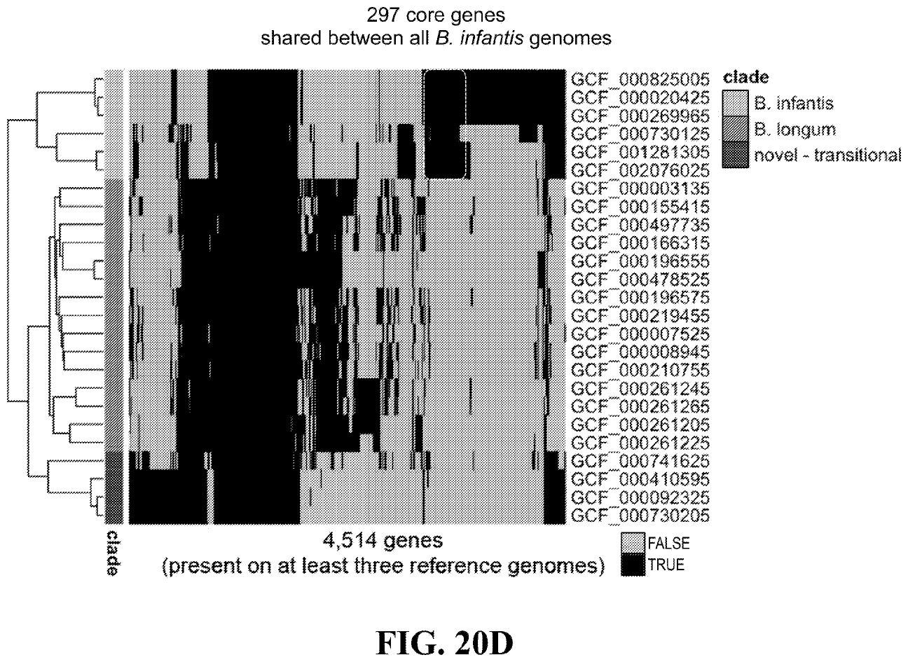

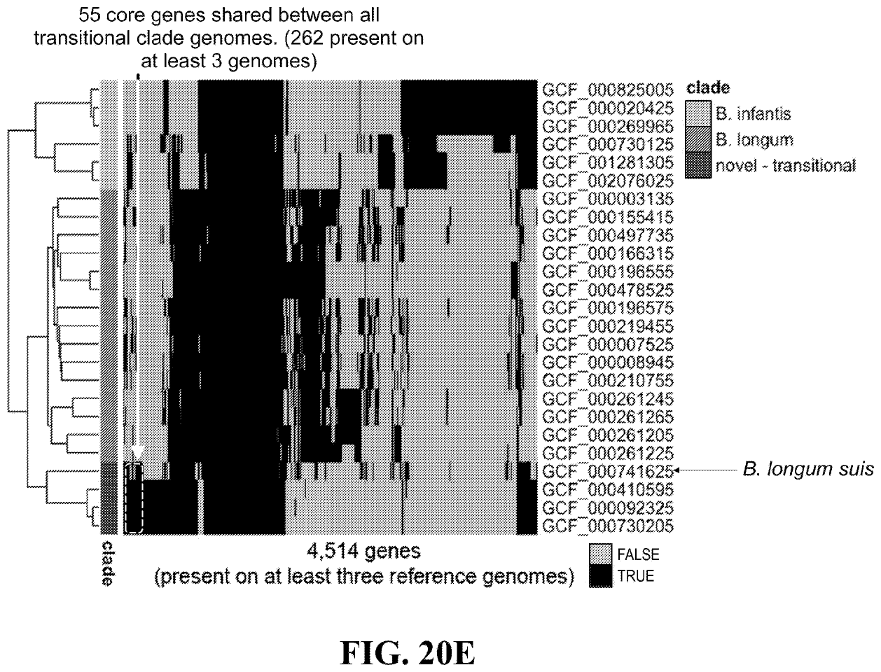

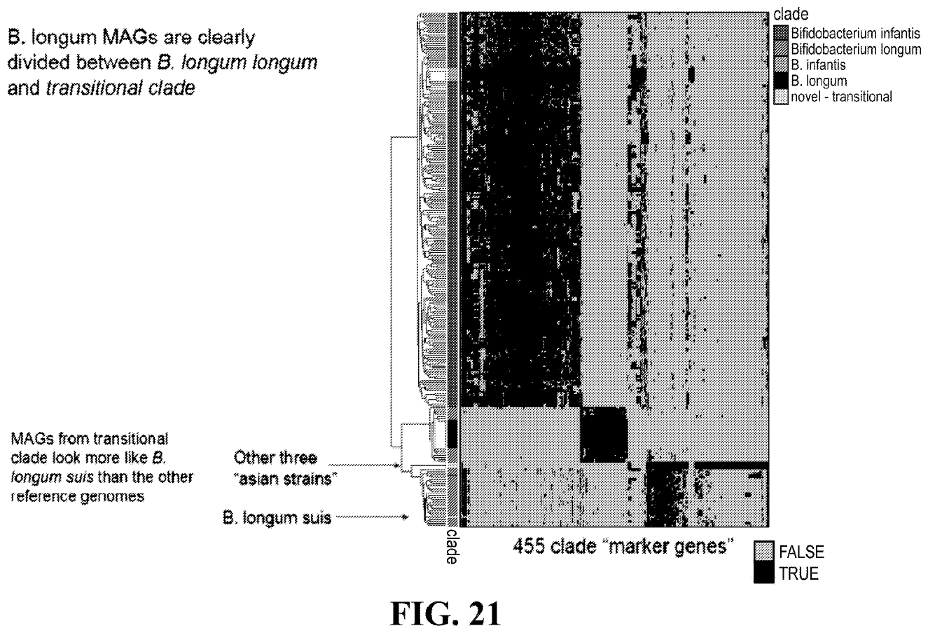

Overall, 234 HQ MAGs were labeled as B. longum subspecies longum or B. longum subspecies infantis. A pangenome was built together with 25 B. longum subspecies longum/B. longum subspecies infantis reference genomes. 259 “unique” genomes in total. Genomes harbored 484,213 genes in total (sum of genes on all genomes). Genes with >95% identity were considered non-redundant genes. The pangenome harbored 13,834 non-redundant genes. (FIG. 19). Three clades were clearly separated on reference genomes (FIGS. 20A-20E). Further, B. longum MAGs are clearly divided between B. longum subspecies longum and a B. longum transitional clade. MAGs from the B. longum transitional clade look more like B. longum subspecies suis than other reference genomes (FIG. 21).

### Example 3—Tracking Marker Genes in the MAGs

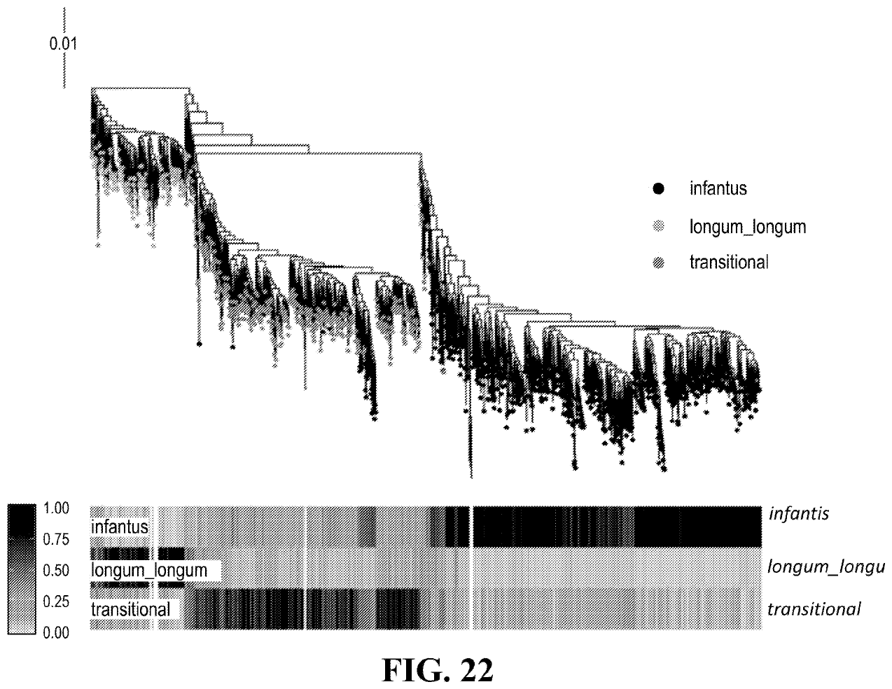

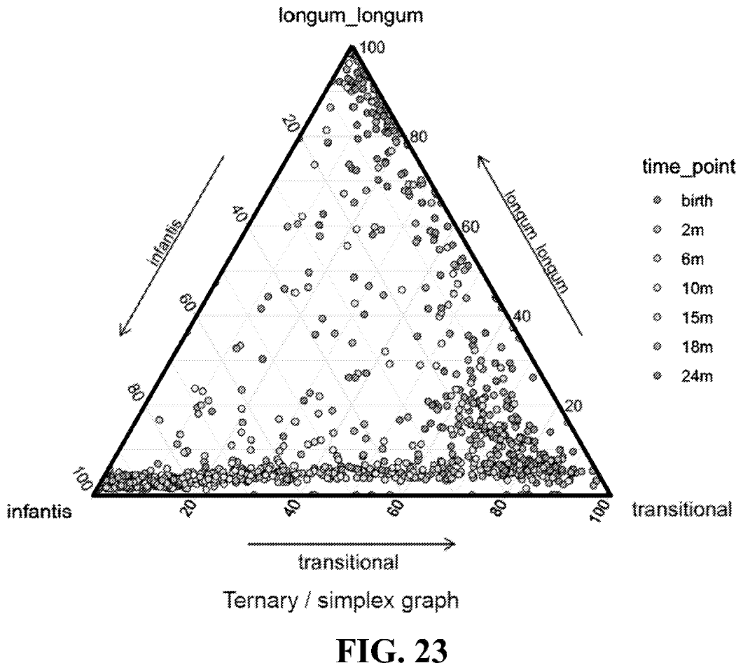

Mapping from marker genes to the B. longum pangenome/UniRef90 gene family IDs in ChocoPhlAn was completed. The quality of the marker genes was assessed by covariance, prevalence, and co-clustering analyses. The most prevalent marker genes per clade were used to estimate clade abundance (within B. longum). Abundance of the clade=c=m_{c}/sum[m_{c}]_{c=1.3}, where me is the median of the clade specific marker gene CPM values (from HUMAnN2 gene family table). FIG. 22 shows a phylogeny and heat map generated using the most prevalent marker genes to estimate and show clade abundance. Points on the phylogeny show the dominant strain by marker gene abundance. The heatmap shows clade abundance within B. longum. FIG. 23 shows a ternary/simplex graph showing the succession from B. subspecies infantis to B. longum “transitional” clade to B. longum subspecies longum. B. longum subspecies infantis and B. longum “transitional” clade coexist while B. longum subspecies infantis and B. longum subspecies longum do not.

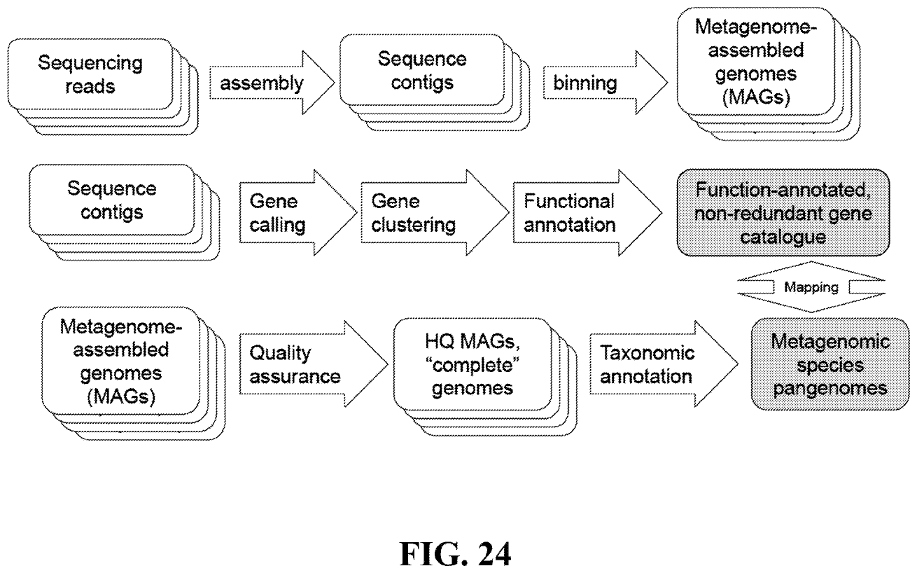

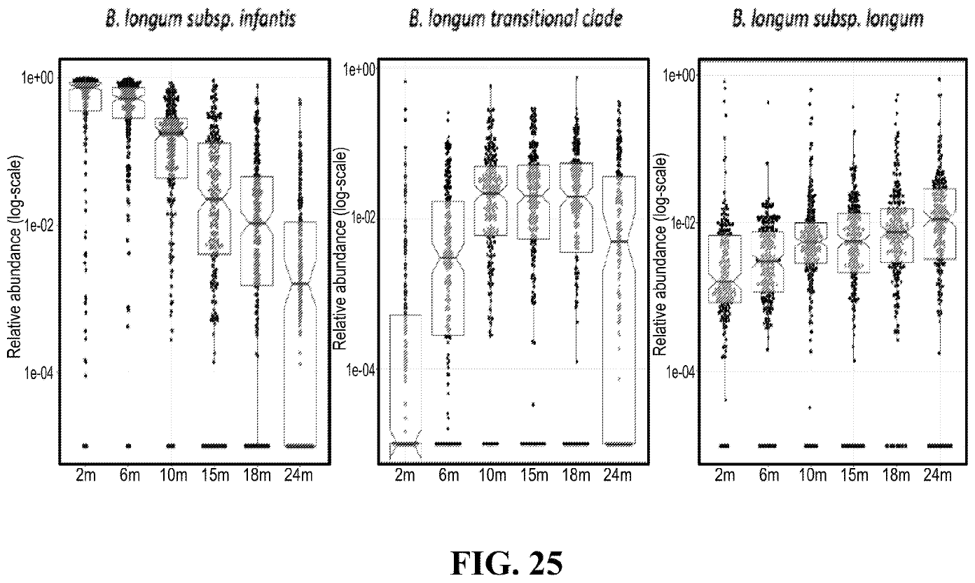

The sequencing reads, contigs and MAGs can be used in a comprehensive, taxonomy-aware functional annotation of the assemblies as shown in FIG. 24. As can be gleaned from FIG. 23, FIG. 25 illustrates the distinct longitudinal trends in B. longum subspecies as revealed by the metagenomic analyses herein.

### Example 4—B. longum Pangenome

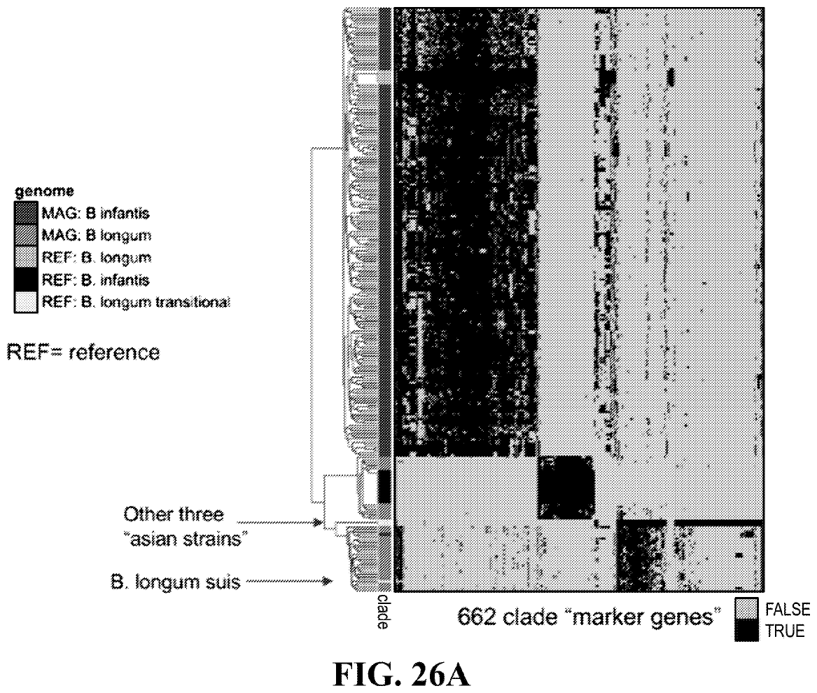

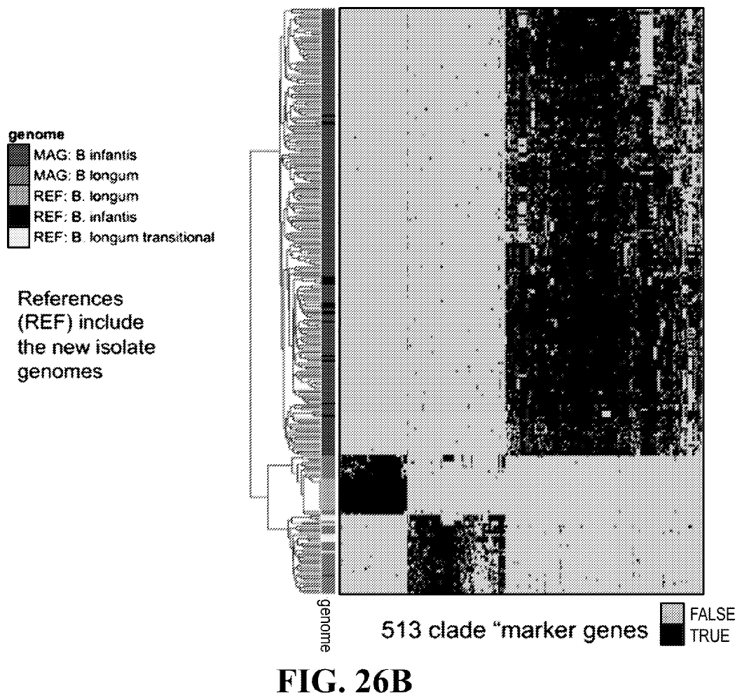

A B. longum pangenome was assembled from reference, isolate, and metagenome assembled genomes (MAGs). 234 high quality MAGs were labeled as B. longum subspecies longum or B. longum subspecies infantis by GTDB-Tk. 16 additional B. longum isolate genomes were included (“NCC” isolates). n=5 novel clade, n=10 B. longum subspecies infantis, n=6 by PacBio sequencing, rest Illumina, n=1 bifidum “control” sequence. 25 reference genomes were also included. The pangenome was built by cd-hit gene clustering (>95% identity). 276 “unique” genomes/MAGs in total were included. Genomes harbored 521, 102 genes in total (sum of all genes on all genomes). 484,213 genes in total before the addition of the isolate genomes to the analysis. The pangenome harbored 15,532 non-redundant genes. The pangenome harbored 13,834 non-redundant genes before the inclusion of the isolate genomes to the analysis. Results are shown in—26A-26B. Three clades clearly separated on reference genomes. MAGs from B. longum transitional clade appeared more like B. longum subspecies suis than the other reference genomes (FIG. 26A).

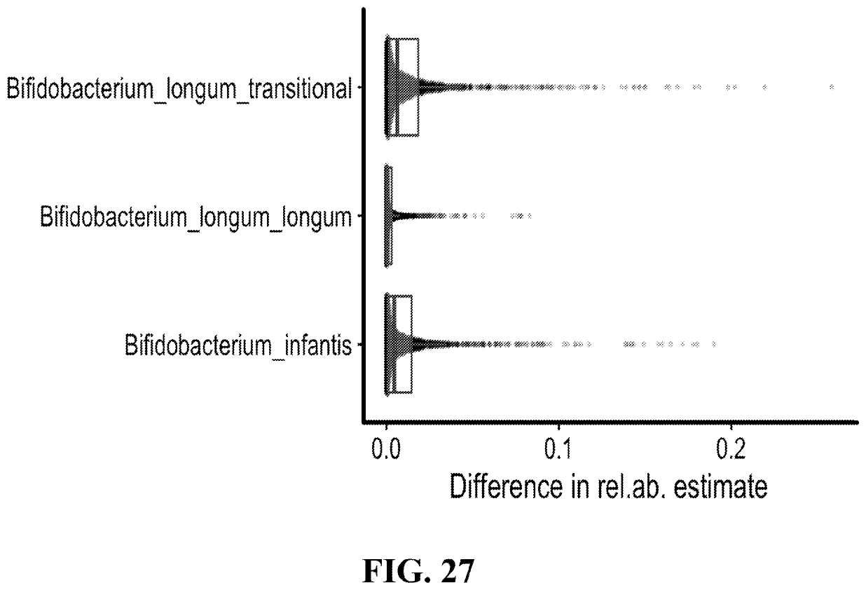

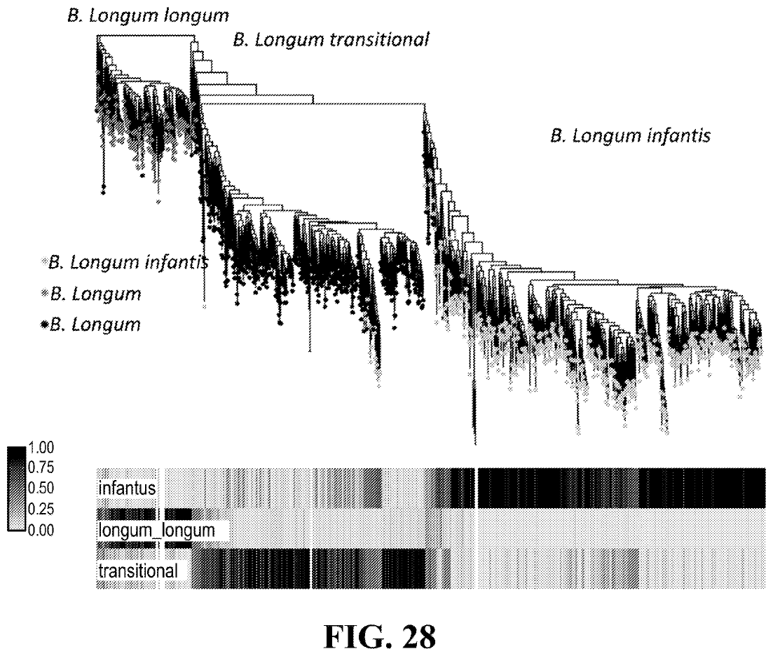

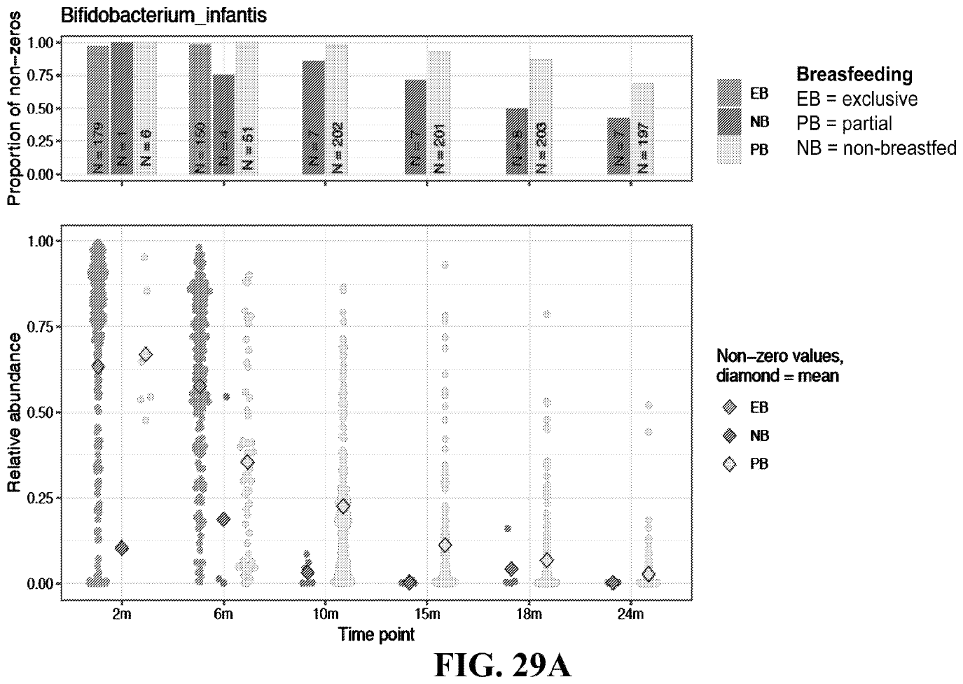

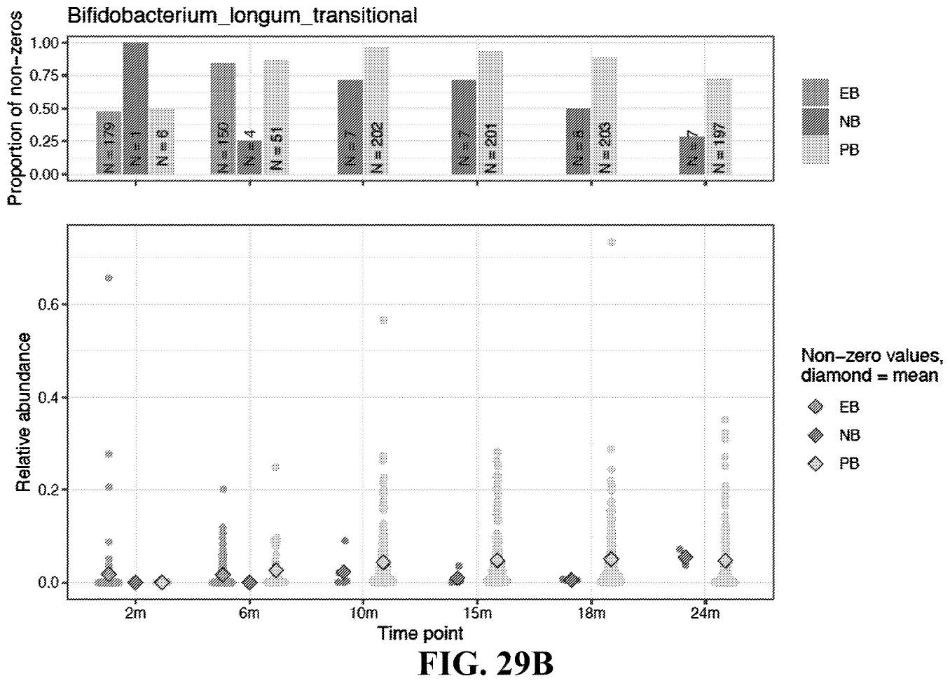

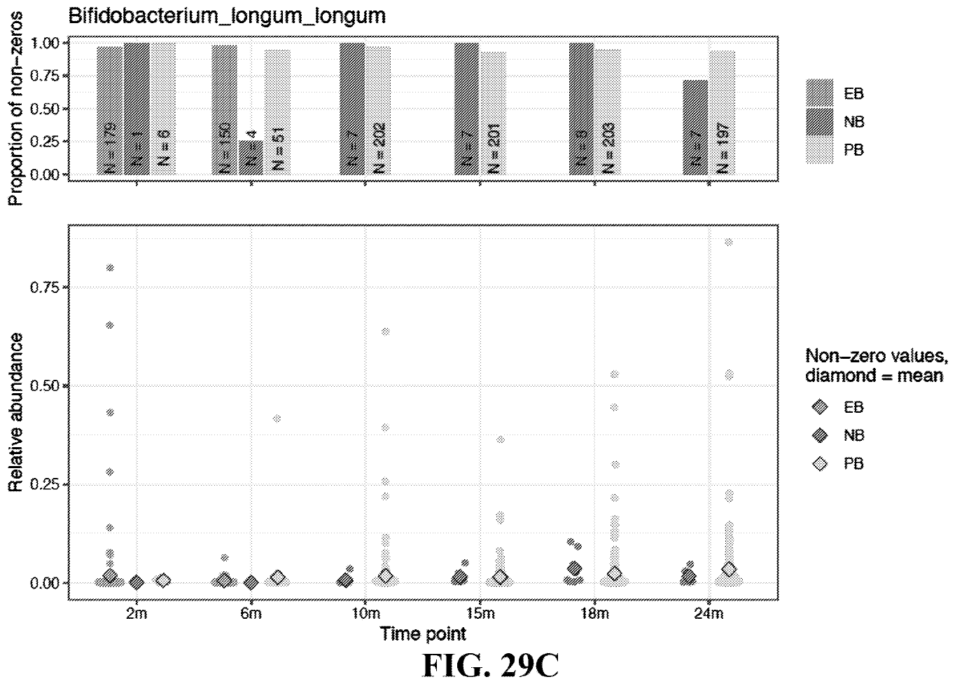

FIG. 27 shows a comparison of abundance estimates between previous MAG analysis data (see e.g., FIGS. 20A-20E, Example 2). Previous marker gene analysis done using MAGs and reference genomes. Most marker genes were shared between the two methods but obtaining new isolate genomes pruned off some “noisy”, unreliable markers. Of the new reduced set of markers, most were concordant between the two methods. 90% of B. longum subspecies longum, 80% of B. longum transitional clade, and 73% of B. longum subspecies infantis marker genes (after obtaining the additional isolates noted in Example 4) were concordant between the two methods. See also FIG. 27. As before (Example 3), marker genes were tracked in the metagenomes using the approach as before (see Example 3). FIG. 28 shows the resulting phylogeny and heat map. The same clear longitudinal distinction between the clades was observed (see e.g., FIGS. 23 and 25). FIGS. 29A-29C show the relative abundance of B. longum subspecies infantis (FIG. 29A), B. longum transitional clade (FIG. 29B), and B. longum subspecies longum (FIG. 29C), which can be compared to results shown in Example 3.

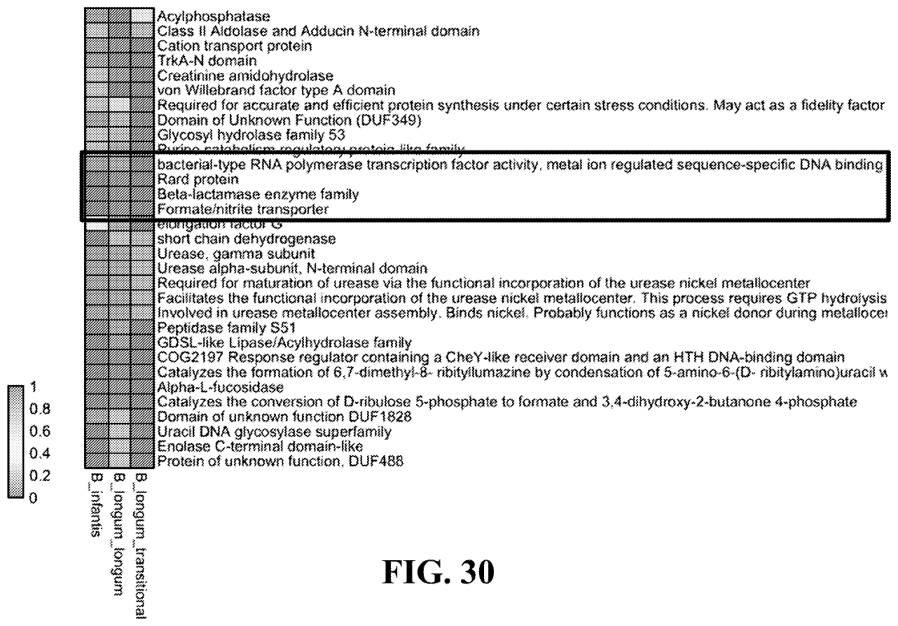

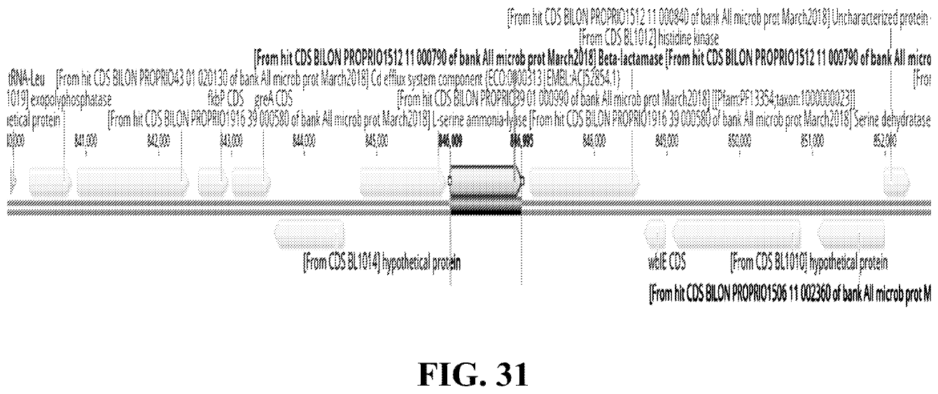

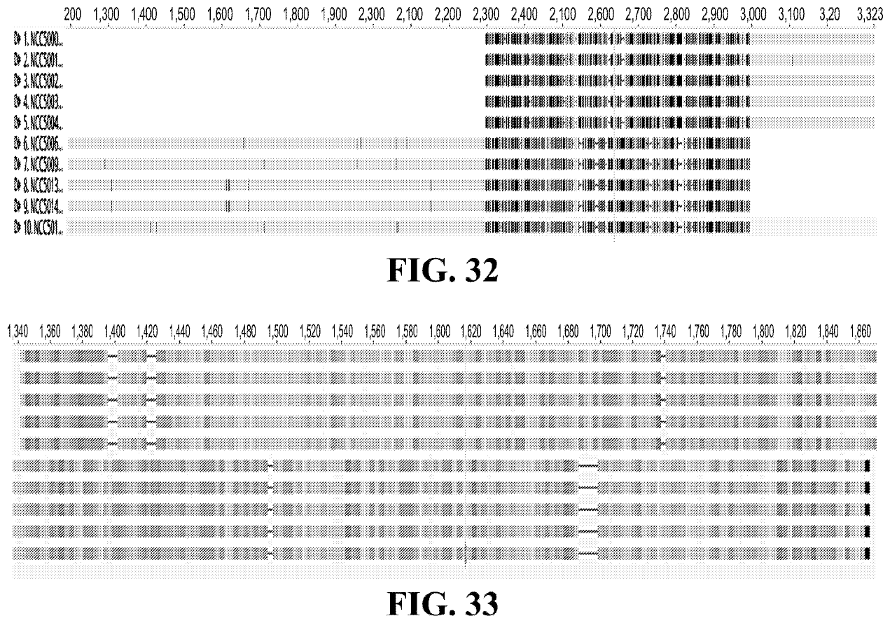

A gene function analysis was done. FIG. 30 shows a heatmap of selected clade-specific gene functions among isolate genomes (or B. longum subspecies longum references). FIG. 31 shows a neighborhood of beta-lactamase gene on a B. longum transitional clade isolate. FIG. 32 shows a nucleotide alignment of B. longum isolates. The overlapping region in the end of the genes (top is B. longum transitional clade beta-lactamase genes, bottom is B. longum subspecies infantis beta-lactamase genes) on nucleotide level (indicates match). The average nucleotide identity was 52% between the clades. FIG. 33 shows an amino acid alignment of B. longum isolates. Overlapping region on grayscale by amino acid seq. Amino acid sequences are distinct per subspecies clades. The average amino acid identity was 34% between the clades.

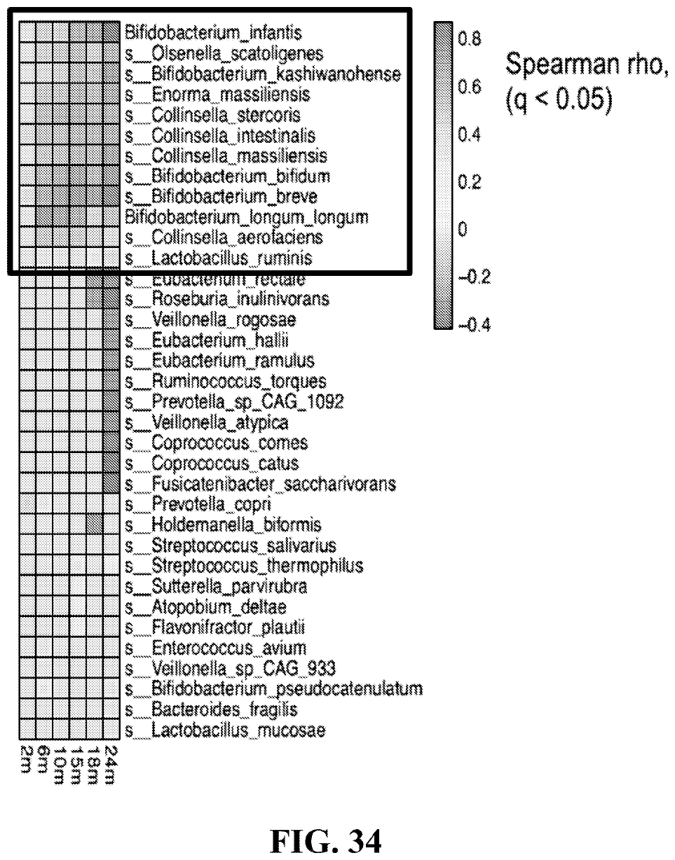

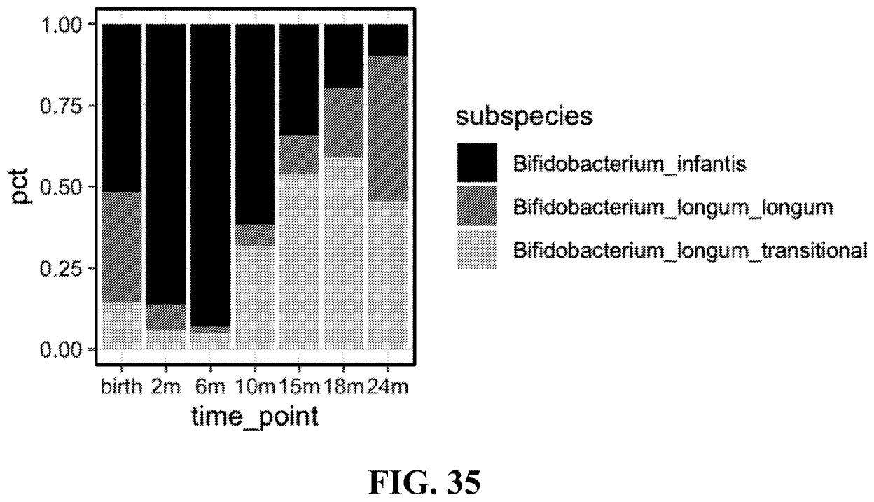

FIG. 34 shows a heatmap showing significant rank-correlations between B. longum transitional clade and other species. FIG. 35 shows the relative abundances of Bifidobacterium longum

FIG. 36 shows UPGMA tree built from ANI distances computed with OrthoANIu software showing separation of the Bifidobacterium subspecies.

### Example 5—B. longum Transitional Clade Marker Genes

A marker gene analysis as previously described was performed using 4 reference genomes (B. longum subspecies suis strain LMG21814 (considered an “outlier” by gene content), 3 Asian strains (B. longum strain BXY01, B. longum subspecies longum CMCC P0001, and B. longum subspecies longum JDM301), 5 NCC isolates (NCC5000, NCC5001, NCC5002, NCC5003, and NCC5004, corresponding to CNCM I-5683, CNCM I-5684, CNCM I-5685, CNCM I-5686 and CNCM I-5687, respectively). For the previous examples herein, the definition of a marker gene was “a gene present on at least 3 out of 4 genomes, which resulted in 262 B. longum transitional clade maker genes, n=55 marker genes if B. longum subspecies suis was included). In this example, the marker gene definition was “a gene present on at least 5 of the 9 reference genomes” (which are noted above). Those maker genes are shown in Table 1. A subgroup of marker genes specific to the NCC isolates was generated. The marker gene definition for this specific group is a gene that is present on all NCC reference isolate (e.g., NCC5000, NCC5001, NCC5002, NCC5003, and NCC5004, corresponding to CNCM I-5683, CNCM I-5684, CNCM I-5685, CNCM I-5686 and CNCM I-5687, respectively) genomes. This subgroup is of genes in presented in Table 2.

Tables 1 and 2 shows B. longum transitional clade marker gene groups. Table 1 shows genes that are present on at least 5 of 9 reference genomes. Table 2 shows genes that are present in all 5 NCC isolate genomes (NCC5000, NCC5001, NCC5002, NCC5003, and NCC5004, corresponding to CNCM I-5683, CNCM I-5684, CNCM I-5685, CNCM I-5686 and CNCM I-5687, respectively). A gene is considered present if it is has a coverage of 90% and has at least 95% shared sequence identity.

TABLE 1
B. longum transitional clade specific genes

| | | | | Presence in n |
|---|---|---|---|---|
| | | | Presence in n | genome from |
| Gene Cluster | Genome | Locus Tag | NCC genome | any clade |
| Cluster 10535 | NCC5001, | 1_1027, 1_983, 1_1011, 1_1056, 1_1038 | 5 | 8 |
| | NCC5000, | | | |
| | NCC5002, | | | |
| | NCC5004, | | | |
| | NCC5003 | | | |
| Cluster 10742 | NCC5003, | 1_1460, 1_628, 1_1449, 1_623, 1_1439 | 5 | 5 |
| | NCC5001, | | | |
| | NCC5002, | | | |
| | NCC5004, | | | |
| | NCC5000 | | | |
| Cluster 10783 | NCC5002, | 1_1489, 1_1495, 1_588, 1_1479 | 4 | 7 |
| | NCC5003, | | | |
| | NCC5004, | | | |
| | NCC5000 | | | |
| Cluster 10912 | NCC5001, | 1_956, 1_1086, 1_1049, 1_981, 1_1113 | 5 | 5 |
| | NCC5002, | | | |
| | NCC5000, | | | |
| | NCC5004, | | | |
| | NCC5003 | | | |
| Cluster 10987 | NCC5002, | 1_281, 1_324 | 2 | 5 |
| | NCC5000 | | | |
| Cluster 12024 | NCC5003, | 1_1172, 1_1106, 1_1145 | 3 | 6 |
| | NCC5000, | | | |
| | NCC5002 | | | |
| Cluster 12123 | NCC5004, | 1_1096, 1_998, 1_1067, 1_971, 1_942 | 5 | 5 |
| | NCC5003, | | | |
| | NCC5001, | | | |
| | NCC5002, | | | |
| | NCC5000 | | | |
| Cluster 12245 | NCC5002, | 1_1087, 1_955, 1_980, 1_1114, 1_1050 | 5 | 5 |
| | NCC5001, | | | |
| | NCC5004, | | | |
| | NCC5003, | | | |
| | NCC5000 | | | |
| Cluster 12381 | NCC5002, | 1_568, 1_1520, 1_650, 1_1438, 1_571 | 5 | 5 |
| | NCC5001, | | | |
| | NCC5003, | | | |
| | NCC5004, | | | |
| | NCC5000 | | | |
| Cluster 1274 | NCC5004, | 1_501, 1_1575, 1_1583, 1_532, 1_1561 | 5 | 5 |
| | NCC5002, | | | |
| | NCC5003, | | | |
| | NCC5001, | | | |
| | NCC5000 | | | |
| Cluster 1378 | NCC5001, | 1_535, 1_1558, 1_1572, 1_504, 1_1580 | 5 | 5 |
| | NCC5000, | | | |
| | NCC5002, | | | |
| | NCC5004, | | | |
| | NCC5003 | | | |
| Cluster 13922 | NCC5003, | 1_997, 1_1068, 1_941, 1_970, 1_1097 | 5 | 5 |
| | NCC5001, | | | |
| | NCC5000, | | | |
| | NCC5002, | | | |
| | NCC5004 | | | |
| Cluster 13960 | NCC5002, | 1_1055, 1_1012, 1_1018, 1_987, 1_1082 | 5 | 8 |
| | NCC5004, | | | |
| | NCC5000, | | | |
| | NCC5001, | | | |
| | NCC5003 | | | |
| Cluster 14054 | NCC5001, | 1_1835, 1_307, 1_1760, 1_277, 1_238 | 5 | 5 |
| | NCC5003, | | | |
| | NCC5004, | | | |
| | NCC5000, | | | |
| | NCC5002 | | | |
| Cluster 143 | NCC5003, | 1_542, 1_464, 1_1545 | 3 | 6 |
| | NCC5002, | | | |
| | NCC5004 | | | |
| Cluster 14471 | NCC5001, | 1_1066, 1_1095, 1_943, 1_999, 1_972 | 5 | 5 |
| | NCC5004, | | | |
| | NCC5000, | | | |
| | NCC5003, | | | |
| | NCC5002 | | | |
| Cluster 1456 | NCC5002, | 1_447, 1_1562, 1_521 | 3 | 5 |
| | NCC5004, | | | |
| | NCC5003 | | | |
| Cluster 14959 | NCC5004, | 1_1126, 1_942, 1_967, 1_892, 1_1098 | 5 | 8 |
| | NCC5002, | | | |
| | NCC5003, | | | |
| | NCC5000, | | | |
| | NCC5001 | | | |
| Cluster 1704 | NCC5001, | 1_1519, 1_1437, 1_651, 1_572, 1_569 | 5 | 5 |
| | NCC5004, | | | |
| | NCC5003, | | | |
| | NCC5000, | | | |
| | NCC5002 | | | |
| Cluster 2040 | NCC5000, | 1_1559, 1_534, 1_503, 1_1581, 1_1573 | 5 | 5 |
| | NCC5001, | | | |
| | NCC5004, | | | |
| | NCC5003, | | | |
| | NCC5002 | | | |
| Cluster 250 | NCC5000, | 1_1298, 1_1284, 1_1311, 1_789 | 4 | 7 |
| | NCC5002, | | | |
| | NCC5003, | | | |
| | NCC5004 | | | |
| Cluster 2546 | NCC5003, | 1_360, 1_325, 1_1708, 1_282 | 4 | 7 |
| | NCC5000, | | | |
| | NCC5004, | | | |
| | NCC5002 | | | |
| Cluster 2563 | NCC5000, | 1_1829, 1_1821, 1_235, 1_257, 1_1852 | 5 | 8 |
| | NCC5003, | | | |
| | NCC5001, | | | |
| | NCC5004, | | | |
| | NCC5002 | | | |
| Cluster 2765 | NCC5000, | 1_320, 1_355, 1_1713, 1_277 | 4 | 7 |
| | NCC5003, | | | |
| | NCC5004, | | | |
| | NCC5002 | | | |
| Cluster 2801 | NCC5001, | 1_1506, 1_582 | 2 | 5 |
| | NCC5000 | | | |
| Cluster 2839 | NCC5002, | 1_453, 1_531, 1_1556 | 3 | 6 |
| | NCC5003, | | | |
| | NCC5004 | | | |
| Cluster 3126 | NCC5004, | 1_616, 1_1467, 1_1461, 1_1447 | 4 | 7 |
| | NCC5003, | | | |
| | NCC5002, | | | |
| | NCC5000 | | | |
| Cluster 320 | NCC5004, | 1_226, 1_1850, 1_206, 1_1882, 1_1879 | 5 | 8 |
| | NCC5003, | | | |
| | NCC5001, | | | |
| | NCC5002, | | | |
| | NCC5000 | | | |
| Cluster 3358 | NCC5001, | 1_199, 1_1857, 1_1886, 1_219, 1_1889 | 5 | 8 |
| | NCC5003, | | | |
| | NCC5000, | | | |
| | NCC5004, | | | |
| | NCC5002 | | | |
| Cluster 3512 | NCC5001, | 1_201, 1_1855, 1_221, 1_1884, 1_1887 | 5 | 8 |
| | NCC5003, | | | |
| | NCC5004, | | | |
| | NCC5000, | | | |
| | NCC5002 | | | |
| Cluster 3753 | NCC5003, | 1_525, 1_446, 1_1563 | 3 | 5 |
| | NCC5002, | | | |
| | NCC5004 | | | |
| Cluster 4705 | NCC5004, | 1_615, 1_1448, 1_1462, 1_1468 | 4 | 7 |
| | NCC5000, | | | |
| | NCC5002, | | | |
| | NCC5003 | | | |
| Cluster 5109 | NCC5004, | 1_1435, 1_571, 1_653, 1_574, 1_1517 | 5 | 8 |
| | NCC5002, | | | |
| | NCC5003, | | | |
| | NCC5000, | | | |
| | NCC5001 | | | |
| Cluster 5197 | NCC5004, | 1_218, 1_1887, 1_1890, 1_1858 | 4 | 7 |
| | NCC5000, | | | |
| | NCC5002, | | | |
| | NCC5003 | | | |
| Cluster 5301 | NCC5003, | 1_1000, 1_1094, 1_973, 1_1065, 1_944 | 5 | 5 |
| | NCC5004, | | | |
| | NCC5002, | | | |
| | NCC5001, | | | |
| | NCC5000 | | | |
| Cluster 5401 | NCC5001, | 1_51, 1_2023, 1_1365, 1_1338 | 4 | 6 |
| | NCC5000, | | | |
| | NCC5003, | | | |
| | NCC5002 | | | |
| Cluster 5664 | NCC5002, | 1_276, 1_1714, 1_354, 1_319 | 4 | 7 |
| | NCC5004, | | | |
| | NCC5003, | | | |
| | NCC5000 | | | |
| Cluster 5716 | NCC5004, | 1_1488, 1_1574, 1_518, 1_599, 1_521_{—} | 5 | 8 |
| | NCC5001, | | | |
| | NCC5002, | | | |
| | NCC5003, | | | |
| | NCC5000 | | | |
| Cluster 5827 | NCC5002, | 1_1460, 1_617, 1_1446, 1_1466 | 4 | 7 |
| | NCC5004, | | | |
| | NCC5000, | | | |
| | NCC5003 | | | |
| Cluster 6058 | NCC5003, | 1_223, 1_217, 1_1847 | 3 | 6 |
| | NCC5000, | | | |
| | NCC5004 | | | |
| Cluster 6120 | NCC5003, | 1_529, 1_1559, 1_450 | 3 | 6 |
| | NCC5004, | | | |
| | NCC5002 | | | |
| Cluster 6142 | NCC5004, | 1_1558, 1_451 | 2 | 5 |
| | NCC5002 | | | |
| Cluster 6148 | NCC5004, | 1_2050, 1_14, 1_15, 1_2096, 1_14 | 5 | 7 |
| | NCC5000, | | | |
| | NCC5003, | | | |
| | NCC5001, | | | |
| | NCC5002 | | | |
| Cluster 6168 | NCC5003, | 1_600, 1_519, 1_1573, 1_522, 1_1487 | 5 | 8 |
| | NCC5002, | | | |
| | NCC5001, | | | |
| | NCC5000, | | | |
| | NCC5004 | | | |
| Cluster 6333 | NCC5003, | 1_1465, 1_618, 1_1459, 1_1445 | 4 | 7 |
| | NCC5004, | | | |
| | NCC5002, | | | |
| | NCC5000 | | | |
| Cluster 6443 | NCC5003, | 1_1853, 1_203, 1_1882, 1_223, 1_1885 | 5 | 8 |
| | NCC5001, | | | |
| | NCC5000, | | | |
| | NCC5004, | | | |
| | NCC5002 | | | |
| Cluster 6500 | NCC5004, | 1_63, 1_2050, 1_2011 | 3 | 5 |
| | NCC5002, | | | |
| | NCC5003 | | | |
| Cluster 6584 | NCC5000, | 1_318, 1_353, 1_275, 1_1715 | 4 | 7 |
| | NCC5003, | | | |
| | NCC5002, | | | |
| | NCC5004 | | | |
| Cluster 7251 | NCC5001, | 1_1500, 1_587 | 2 | 5 |
| | NCC5000 | | | |
| Cluster 7435 | NCC5002, | 1_1888, 1_220, 1_200, 1_1856, 1_1885 | 5 | 8 |
| | NCC5004, | | | |
| | NCC5001, | | | |
| | NCC5003, | | | |
| | NCC5000 | | | |
| Cluster 7556 | NCC5003, | 1_813, 1_1279, 1_737 | 3 | 6 |
| | NCC5004, | | | |
| | NCC5000 | | | |
| Cluster 7961 | NCC5004, | 1_1102, 1_968, 1_932, 1_1070, 1_992 | 5 | 5 |
| | NCC5002, | | | |
| | NCC5000, | | | |
| | NCC5001, | | | |
| | NCC5003 | | | |
| Cluster 8187 | NCC5001, | 1_2095, 1_15, 1_2049, 1_16, 1_15 | 5 | 8 |
| | NCC5002, | | | |
| | NCC5004, | | | |
| | NCC5003, | | | |
| | NCC5000 | | | |
| Cluster 8251 | NCC5002, | 1_21, 1_2087, 1_51, 1_2013, 1_49 | 5 | 5 |
| | NCC5001, | | | |
| | NCC5003, | | | |
| | NCC5004, | | | |
| | NCC5000 | | | |
| Cluster 8370 | NCC5000, | 1_581, 1_1507 | 2 | 5 |
| | NCC5001 | | | |
| Cluster 8376 | NCC5000, | 1_619, 1_1389, 1_618, 1_1449, 1_699 | 5 | 8 |
| | NCC5004, | | | |
| | NCC5002, | | | |
| | NCC5001, | | | |
| | NCC5003 | | | |
| Cluster 889 | NCC5000, | 1_1563, 1_1585, 1_499, 1_1577 | 4 | 7 |
| | NCC5003, | | | |
| | NCC5004, | | | |
| | NCC5002 | | | |
| Cluster 9183 | NCC5001, | 1_1505, 1_583 | 2 | 5 |
| | NCC5000 | | | |
| Cluster 9218 | NCC5004, | 1_1553, 1_456 | 2 | 5 |
| | NCC5002 | | | |
| Cluster 9634 | NCC5001, | 1_1615, 1_474, 1_553, 1_1534 | 4 | 7 |
| | NCC5002, | | | |
| | NCC5003, | | | |
| | NCC5004 | | | |
| Cluster 980 | NCC5000, | 1_1444, 1_619, 1_1458, 1_1464 | 4 | 7 |
| | NCC5004, | | | |
| | NCC5002, | | | |
| | NCC5003 | | | |

| | Predicted_pro- | | | |
|---|---|---|---|---|
| Gene Cluster | tein_name | Gene_Ontology_terms | EC_number | KEGG_ko |
| Cluster 10535 | | | | |
| Cluster 10742 | | | | |
| Cluster 10783 | | | | ko: K17713 |
| Cluster 10912 | mepA_6 | | | |
| Cluster 10987 | fucU | GO: 0003674, GO: 0003824, GO: 0005488, GO: 0005575, GO: 0005622, | 5.1.3.29 | ko: K02431 |
| | | GO: 0005623, GO: 0005737, GO: 0016853, GO: 0016854, GO: 0016857, | | |
| | | GO: 0030246, GO: 0036094, GO: 0042806, GO: 0044424, GO: 0044464, | | |
| | | GO: 0048029 | | |
| Cluster 12024 | | | 2.7.11.1 | ko: K07154 |
| Cluster 12123 | | | | |
| Cluster 12245 | | | | |
| Cluster 12381 | | | 3.2.1.23 | ko: K01190 |
| Cluster 1274 | | | | |
| Cluster 1378 | cotH | | | ko: K06330 |
| Cluster 13922 | | | 2.1.1.72, | ko: K01153, |
| | | | 3.1.21.3, | ko: K01155, |
| | | | 3.1.21.4 | ko: K03427, |
| | | | | ko: K09384 |
| Cluster 13960 | | | | |
| Cluster 14054 | atpE | GO: 0003674, GO: 0003824, GO: 0005215, GO: 0005575, GO: 0005622, | | ko: K02110 |
| | | GO: 0005623, GO: 0006139, GO: 0006163, GO: 0006164, GO: 0006725, | | |
| | | GO: 0006753, GO: 0006754, GO: 0006793, GO: 0006796, GO: 0006807, | | |
| | | GO: 0006810, GO: 0006811, GO: 0006812, GO: 0008150, GO: 0008152, | | |
| | | GO: 0008324, GO: 0009058, GO: 0009117, GO: 0009123, GO: 0009124, | | |
| | | GO: 0009126, GO: 0009127, GO: 0009141, GO: 0009142, GO: 0009144, | | |
| | | GO: 0009145, GO: 0009150, GO: 0009152, GO: 0009156, GO: 0009161, | | |
| | | GO: 0009165, GO: 0009167, GO: 0009168, GO: 0009199, GO: 0009201, | | |
| | | GO: 0009205, GO: 0009206, GO: 0009259, GO: 0009260, GO: 0009987, | | |
| | | GO: 0015075, GO: 0015077, GO: 0015078, GO: 0015318, GO: 0015399, | | |
| | | GO: 0015405, GO: 0015672, GO: 0015985, GO: 0015986, GO: 0016020, | | |
| | | GO: 0016462, GO: 0016469, GO: 0016787, GO: 0016817, GO: 0016818, | | |
| | | GO: 0016887, GO: 0017111, GO: 0017144, GO: 0018130, GO: 0019438, | | |
| | | GO: 0019637, GO: 0019693, GO: 0019829, GO: 0022804, GO: 0022853, | | |
| | | GO: 0022857, GO: 0022890, GO: 0032991, GO: 0033177, GO: 0034220, | | |
| | | GO: 0034641, GO: 0034654, GO: 0042623, GO: 0042625, GO: 0042626, | | |
| | | GO: 0043492, GO: 0044237, GO: 0044238, GO: 0044249, GO: 0044271, | | |
| | | GO: 0044281, GO: 0044424, GO: 0044425, GO: 0044464, GO: 0044769, | | |
| | | GO: 0045259, GO: 0045263, GO: 0046034, GO: 0046390, GO: 0046483, | | |
| | | GO: 0046933, GO: 0051179, GO: 0051234, GO: 0055085, GO: 0055086, | | |
| | | GO: 0071704, GO: 0072521, GO: 0072522, GO: 0090407, GO: 0090662, | | |
| | | GO: 0098655, GO: 0098660, GO: 0098662, GO: 0098796, GO: 0099131, | | |
| | | GO: 0099132, GO: 1901135, GO: 1901137, GO: 1901293, GO: 1901360, | | |
| | | GO: 1901362, GO: 1901564, GO: 1901566, GO: 1901576, GO: 1902600 | | |
| Cluster 143 | uvrD | | 3.6.4.12 | ko: K03657 |
| Cluster 14471 | | | | |
| Cluster 1456 | | | | |
| Cluster 14959 | mepA | | | ko: K18908 |
| Cluster 1704 | aldH | | 1.2.1.3, | ko: K00128, |
| | | | 1.2.1.5 | ko: K00129 |
| Cluster 2040 | pelG | | | ko: K21012 |
| Cluster 250 | | | | |
| Cluster 2546 | fucA | | 3.2.1.51 | ko: K01206 |
| Cluster 2563 | | | | |
| Cluster 2765 | | | | ko: K02027 |
| Cluster 2801 | | | | ko: K03315 |
| Cluster 2839 | | | | ko: K07133 |
| Cluster 3126 | | | | ko: K02027, |
| | | | | ko: K17329 |
| Cluster 320 | rne | GO: 0006139, GO: 0006364, GO: 0006396, GO: 0006397, GO: 0006725, | 3.1.26.12 | ko: K08300, |
| | | GO: 0006807, GO: 0008150, GO: 0008152, GO: 0009987, GO: 0010467, | | ko: K08301 |
| | | GO: 0016070, GO: 0016071, GO: 0016072, GO: 0022613, GO: 0034470, | | |
| | | GO: 0034641, GO: 0034660, GO: 0042254, GO: 0043170, GO: 0044085, | | |
| | | GO: 0044237, GO: 0044238, GO: 0046483, GO: 0071704, GO: 0071840, | | |
| | | GO: 0090304, GO: 1901360 | | |
| Cluster 3358 | | | 4.2.1.68 | ko: K18334 |
| Cluster 3512 | fucP | | | ko: K02429 |
| Cluster 3753 | | | | |
| Cluster 4705 | | | | |
| Cluster 5109 | purM | GO: 0003674, GO: 0003824, GO: 0004641, GO: 0005575, GO: 0005622, | 6.3.3.1, | ko: K01933, |
| | | GO: 0005623, GO: 0005737, GO: 0005829, GO: 0006139, GO: 0006163, | 6.3.4.13 | ko: K11788 |
| | | GO: 0006164, GO: 0006725, GO: 0006753, GO: 0006793, GO: 0006796, | | |
| | | GO: 0006807, GO: 0008150, GO: 0008152, GO: 0009058, GO: 0009117, | | |
| | | GO: 0009150, GO: 0009152, GO: 0009165, GO: 0009259, GO: 0009260, | | |
| | | GO: 0009987, GO: 0016874, GO: 0016879, GO: 0016882, GO: 0018130, | | |
| | | GO: 0019438, GO: 0019637, GO: 0019693, GO: 0034641, GO: 0034654, | | |
| | | GO: 0044237, GO: 0044238, GO: 0044249, GO: 0044271, GO: 0044281, | | |
| | | GO: 0044424, GO: 0044444, GO: 0044464, GO: 0046390, GO: 0046483, | | |
| | | GO: 0055086, GO: 0071704, GO: 0072521, GO: 0072522, GO: 0090407, | | |
| | | GO: 1901135, GO: 1901137, GO: 1901293, GO: 1901360, GO: 1901362, | | |
| | | GO: 1901564, GO: 1901566, GO: 1901576 | | |
| Cluster 5197 | | | | ko: K02529, |
| | | | | ko: K17640 |
| Cluster 5301 | | | | |
| Cluster 5401 | | | | |
| Cluster 5664 | | | | ko: K02026 |
| Cluster 5716 | | | | ko: K02034, |
| | | | | ko: K15582 |
| Cluster 5827 | | | | ko: K02025, |
| | | | | ko: K10118, |
| | | | | ko: K15771, |
| | | | | ko: K17330 |
| Cluster 6058 | icaR | | | |
| Cluster 6120 | | | | |
| Cluster 6142 | | | | |
| Cluster 6148 | | | | |
| Cluster 6168 | oppD | | | ko: K02031 |
| Cluster 6333 | | | | ko: K02026, |
| | | | | ko: K17331 |
| Cluster 6443 | dapA | | 4.1.2.28, | ko: K01714, |
| | | | 4.3.3.7 | ko: K22397 |
| Cluster 6500 | | | | |
| Cluster 6584 | | | | ko: K02025, |
| | | | | ko: K10118, |
| | | | | ko: K15771 |
| Cluster 7251 | | | | |
| Cluster 7435 | | | | |
| Cluster 7556 | | | | |
| Cluster 7961 | | | 3.4.17.13 | ko: K01297 |
| Cluster 8187 | | | | |
| Cluster 8251 | | | | |
| Cluster 8370 | | GO: 0001775, GO: 0002252, GO: 0002263, GO: 0002274, GO: 0002275, | | |
| | | GO: 0002283, GO: 0002366, GO: 0002376, GO: 0002443, GO: 0002444, | | |
| | | GO: 0002446, GO: 0003674, GO: 0003824, GO: 0004033, GO: 0005575, | | |
| | | GO: 0005622, GO: 0005623, GO: 0005737, GO: 0005829, GO: 0005886, | | |
| | | GO: 0005887, GO: 0006139, GO: 0006725, GO: 0006732, GO: 0006733, | | |
| | | GO: 0006739, GO: 0006753, GO: 0006793, GO: 0006796, GO: 0006807, | | |
| | | GO: 0006810, GO: 0006887, GO: 0006955, GO: 0008076, GO: 0008150, | | |
| | | GO: 0008152, GO: 0009117, GO: 0009898, GO: 0009987, GO: 0010959, | | |
| | | GO: 0012505, GO: 0012506, GO: 0015459, GO: 0016020, GO: 0016021, | | |
| | | GO: 0016192, GO: 0016247, GO: 0016491, GO: 0016614, GO: 0016616, | | |
| | | GO: 0019362, GO: 0019637, GO: 0019897, GO: 0019898, GO: 0030141, | | |
| | | GO: 0030424, GO: 0030659, GO: 0030667, GO: 0031090, GO: 0031224, | | |
| | | GO: 0031226, GO: 0031234, GO: 0031410, GO: 0031982, GO: 0032879, | | |
| | | GO: 0032880, GO: 0032940, GO: 0032991, GO: 0033267, GO: 0034641, | | |
| | | GO: 0034702, GO: 0034703, GO: 0034705, GO: 0034762, GO: 0034765, | | |
| | | GO: 0035579, GO: 0036230, GO: 0042119, GO: 0042581, GO: 0042995, | | |
| | | GO: 0043005, GO: 0043226, GO: 0043227, GO: 0043229, GO: 0043266, | | |
| | | GO: 0043269, GO: 0043299, GO: 0043312, GO: 0044224, GO: 0044237, | | |
| | | GO: 0044238, GO: 0044281, GO: 0044304, GO: 0044422, GO: 0044424, | | |
| | | GO: 0044425, GO: 0044433, GO: 0044444, GO: 0044446, GO: 0044459, | | |
| | | GO: 0044463, GO: 0044464, GO: 0045055, GO: 0045321, GO: 0046483, | | |
| | | GO: 0046496, GO: 0046903, GO: 0050789, GO: 0050794, GO: 0050896, | | |
| | | GO: 0051049, GO: 0051179, GO: 0051186, GO: 0051234, GO: 0055086, | | |
| | | GO: 0055114, GO: 0060341, GO: 0065007, GO: 0065009, GO: 0070820, | | |
| | | GO: 0070821, GO: 0070995, GO: 0071704, GO: 0071944, GO: 0072524, | | |
| | | GO: 0097458, GO: 0097708, GO: 0098552, GO: 0098562, GO: 0098588, | | |
| | | GO: 0098772, GO: 0098796, GO: 0098797, GO: 0098805, GO: 0099106, | | |
| | | GO: 0099503, GO: 0120025, GO: 0120038, GO: 1901360, GO: 1901379, | | |
| | | GO: 1901564, GO: 1902495, GO: 1903827, GO: 1904062, GO: 1990031, | | |
| | | GO: 1990351, GO: 2000008 | | |
| Cluster 8376 | magIII | | | ko: K07457 |
| Cluster 889 | | | 5.1.3.2 | ko: K01784 |
| Cluster 9183 | mug | | 3.2.2.28 | ko: K03575, |
| | | | | ko: K03649 |
| Cluster 9218 | | | | |
| Cluster 9634 | | | | |
| Cluster 980 | | | | |

| Gene Cluster | KEGG_Pathway | KEGG_Module | KEGG_Reaction | KEGG_rclass |
|---|---|---|---|---|
| Cluster 10535 | | | | |
| Cluster 10742 | | | | |
| Cluster 10783 | | | | |
| Cluster 10912 | | | | |
| Cluster 10987 | | | R10764 | RC00563 |
| Cluster 12024 | | | | |
| Cluster 12123 | | | | |
| Cluster 12245 | | | | |
| Cluster 12381 | ko00052, ko00511, | | R01105, R01678, | RC00049, RC00452 |
| | ko00600, ko01100, | | R03355, R04783, | |
| | map00052, map00511, | | R06114 | |
| | map00600, map01100 | | | |
| Cluster 1274 | | | | |
| Cluster 1378 | | | | |
| Cluster 13922 | | | | |
| Cluster 13960 | | | | |
| Cluster 14054 | ko00190, ko00195, | M00157 | | |
| | ko01100, map00190, | | | |
| | map00195, map01100 | | | |
| Cluster 143 | ko03420, ko03430, | | | |
| | map03420, map03430 | | | |
| Cluster 14471 | | | | |
| Cluster 1456 | | | | |
| Cluster 14959 | | M00705 | | |
| Cluster 1704 | ko00010, ko00053, | M00135 | R00264, R00631, | RC00047, RC00071, |
| | ko00071, ko00280, | | R00710, R00711, | RC00080, RC00186, |
| | k000310, ko00330, | | R00904, R01752, | RC00218, RC00242, |
| | ko00340, ko00350, | | R01986, R02536, | RC00816, RC01500, |
| | ko00360, ko00380, | | R02537, R02549, | RC01735 |
| | ko00410, ko00561, | | R02678, R02695, | |
| | ko00620, ko00625, | | R02697, R02940, | |
| | ko00903, k000980, | | R02957, R03283, | |
| | ko00981, ko00982, | | R03300, R03302, | |
| | ko01100, ko01110, | | R03869, R04065, | |
| | ko01120, ko01130, | | R04506, R04882, | |
| | ko05204, map00010, | | R04883, R04888, | |
| | map00053, map00071, | | R04889, R04891, | |
| | map00280, map00310, | | R04892, R04903, | |
| | map00330, map00340, | | R04996, R05050, | |
| | map00350, map00360, | | R05237, R05238, | |
| | map00380, map00410, | | R05286, R06366, | |
| | map00561, map00620, | | R07104, R08146, | |
| | map00625, map00903, | | R08282, R08283, | |
| | map00980, map00981, | | R08307 | |
| | map00982, map01100, | | | |
| | map01110, map01120, | | | |
| | map01130, map05204 | | | |
| Cluster 2040 | ko02025, map02025 | | | |
| Cluster 250 | | | | |
| Cluster 2546 | ko00511, map00511 | | | |
| Cluster 2563 | | | | |
| Cluster 2765 | | M00207 | | |
| Cluster 2801 | | | | |
| Cluster 2839 | | | | |
| Cluster 3126 | ko02010, map02010 | M00207, M00606 | | |
| Cluster 320 | ko03018, map03018 | M00394 | | |
| Cluster 3358 | ko00051, ko01120, | | R03688 | RC00543 |
| | map00051, map01120 | | | |
| Cluster 3512 | | | | |
| Cluster 3753 | | | | |
| Cluster 4705 | | | | |
| Cluster 5109 | ko00230, ko01100, | M00048 | R04144, R04208 | RC00090, RC00166, |
| | ko01110, ko01130, | | | RC01100 |
| | map00230, map01100, | | | |
| | map01110, map01130 | | | |
| Cluster 5197 | | | | |
| Cluster 5301 | | | | |
| Cluster 5401 | | | | |
| Cluster 5664 | | M00207 | | |
| Cluster 5716 | ko01501, ko02010, | M00239, M00439 | | |
| | ko02024, map01501, | | | |
| | map02010, map02024 | | | |
| Cluster 5827 | ko02010, map02010 | M00196, M00207, M00491, M00606 | | |
| Cluster 6058 | | | | |
| Cluster 6120 | | | | |
| Cluster 6142 | | | | |
| Cluster 6148 | | | | |
| Cluster 6168 | ko02024, map02024 | M00239 | | |
| Cluster 6333 | ko02010, map02010 | M00207, M00606 | | |
| Cluster 6443 | ko00040, ko00261, | M00016, M00525, M00526, M00527 | R01782, R10147 | RC00307, RC00572, |
| | ko00300, ko01100, | | | RC03062, RC03063 |
| | k001110, ko01120, | | | |
| | ko01130, ko01230, | | | |
| | map00040, map00261, | | | |
| | map00300, map01100, | | | |
| | map01110, map01120, | | | |
| | map01130, map01230 | | | |
| Cluster 6500 | | | | |
| Cluster 6584 | ko02010, map02010 | M00196, M00207, M00491 | | |
| Cluster 7251 | | | | |
| Cluster 7435 | | | | |
| Cluster 7556 | | | | |
| Cluster 7961 | | | | |
| Cluster 8187 | | | | |
| Cluster 8251 | | | | |
| Cluster 8370 | | | | |
| Cluster 8376 | | | | |
| Cluster 889 | ko00052, ko00520, | M00361, M00362, M00632 | R00291, R02984 | RC00289 |
| | ko01100, map00052, | | | |
| | map00520, map01100 | | | |
| Cluster 9183 | ko03410, map03410 | | | |
| Cluster 9218 | | | | |
| Cluster 9634 | | | | |
| Cluster 980 | | | | |

| Gene Cluster | BRITE | KEGG_TC | CAZy | BiGG_Reaction |
|---|---|---|---|---|
| Cluster 10535 | | | | |
| Cluster 10742 | | | | |
| Cluster 10783 | ko00000, ko02000 | 1.B.33.1 | | |
| Cluster 10912 | | | | |
| Cluster 10987 | ko00000, ko01000 | | | |
| Cluster 12024 | ko00000, ko01000, | | | |
| | ko01001, ko02048 | | | |
| Cluster 12123 | | | | |
| Cluster 12245 | | | | |
| Cluster 12381 | ko00000, ko00001, | | | |
| | ko01000 | | | |
| Cluster 1274 | | | | |
| Cluster 1378 | ko00000 | | | |
| Cluster 13922 | ko00000, ko01000, | | | |
| | ko02048 | | | |
| Cluster 13960 | | | | |
| Cluster 14054 | ko00000, ko00001, | 3.A.2.1 | | |
| | ko00002, ko00194 | | | |
| Cluster 143 | ko00000, ko00001, | | | |
| | ko01000, ko03400 | | | |
| Cluster 14471 | | | | |
| Cluster 1456 | | | | |
| Cluster 14959 | ko00000, ko00002, | 2.A.66.1.13 | | |
| | ko01504, ko02000 | | | |
| Cluster 1704 | ko00000, ko00001, | | | |
| | ko00002, ko01000 | | | |
| Cluster 2040 | ko00000, ko00001 | | | |
| Cluster 250 | | | | |
| Cluster 2546 | ko00000, ko00001, | | GH29 | |
| | ko01000, ko04147 | | | |
| Cluster 2563 | | | | |
| Cluster 2765 | ko00000, ko00002, | 3.A.1.1 | | |
| | ko02000 | | | |
| Cluster 2801 | ko00000, ko02000 | 2.A.35 | | |
| Cluster 2839 | ko00000 | | | |
| Cluster 3126 | ko00000, ko00001, | 3.A.1.1, 3.A.1.1.33 | | |
| | ko00002, | | | |
| | ko02000 | | | |
| Cluster 320 | ko00000, ko00001, | | | |
| | ko00002, ko01000, | | | |
| | k003009, ko03019 | | | |
| Cluster 3358 | ko00000, ko00001, | | | |
| | ko01000 | | | |
| Cluster 3512 | ko00000, ko02000 | 2.A.1.7 | | |
| Cluster 3753 | | | | |
| Cluster 4705 | | | | |
| Cluster 5109 | ko00000, ko00001, | | | iAF987.Gmet_1844, |
| | ko00002, ko01000 | | | iECSF_1327.ECSF_2340 |
| Cluster 5197 | ko00000, ko03000 | | | |
| Cluster 5301 | | | | |
| Cluster 5401 | | | | |
| Cluster 5664 | ko00000, ko00002, | 3.A.1.1 | | |
| | ko02000 | | | |
| Cluster 5716 | ko00000, ko00001, | 3.A.1.5, 3.A.1.5.1, 3.A.1.5.18, 3.A.1.5.19, | | |
| | ko00002, ko02000 | 3.A.1.5.25 | | |
| Cluster 5827 | ko00000, ko00001, | 3.A.1.1, 3.A.1.1.16, 3.A.1.1.2, 3.A.1.1.28, | | |
| | ko00002, ko02000 | 3.A.1.1.33 | | |
| Cluster 6058 | | | | |
| Cluster 6120 | | | | |
| Cluster 6142 | | | | |
| Cluster 6148 | | | | |
| Cluster 6168 | ko00000, ko00001, | 3.A.1.5 | | |
| | ko00002, ko02000 | | | |
| Cluster 6333 | ko00000, ko00001, | 3.A.1.1, 3.A.1.1.33 | | |
| | ko00002, ko02000 | | | |
| Cluster 6443 | ko00000, ko00001, | | | |
| | ko00002, ko01000 | | | |
| Cluster 6500 | | | | |
| Cluster 6584 | ko00000, ko00001, | 3.A.1.1, 3.A.1.1.16, 3.A.1.1.2, 3.A.1.1.28 | | |
| | ko00002, ko02000 | | | |
| Cluster 7251 | | | | |
| Cluster 7435 | | | | |
| Cluster 7556 | | | | |
| Cluster 7961 | ko00000, ko01000, | | | |
| | ko01002, ko01011 | | | |
| Cluster 8187 | | | | |
| Cluster 8251 | | | | |
| Cluster 8370 | | | | |
| Cluster 8376 | ko00000 | | | |
| Cluster 889 | ko00000, ko00001, | | | |
| | ko00002, ko01000 | | | |
| Cluster 9183 | ko00000, ko00001, | | | |
| | ko01000, ko03400 | | | |
| Cluster 9218 | | | | |
| Cluster 9634 | | | | |
| Cluster 980 | | | | |

| Gene Cluster | tax_scope | COG_Functional_Category | eggNOG_free_text_description |
|---|---|---|---|
| Cluster 10535 | Bacteria | | |
| Cluster 10742 | Bacteria | K | Bacterial regulatory proteins, |
| | | | tetR family |
| Cluster 10783 | Bacteria | T | Pfam Adenylate and Guanylate |
| | | | cyclase catalytic domain |
| Cluster 10912 | Bacteria | V | MatE |
| Cluster 10987 | Bacteria | G | RbsD/FucU transport protein |
| | | | family |
| Cluster 12024 | Bacteria | S | HipA-like C-terminal domain |
| Cluster 12123 | Bacteria | | |
| Cluster 12245 | | | |
| Cluster 12381 | Bacteria | G | Domain of unknown function |
| | | | (DUF4982) |
| Cluster 1274 | Bacteria | S | Uncharacterised protein |
| | | | conserved in bacteria |
| | | | (DUF2194) |
| Cluster 1378 | Bacteria | M | CotH kinase protein |
| Cluster 13922 | Bacteria | L | N-6 DNA methylase |
| Cluster 13960 | Bacteria | | |
| Cluster 14054 | Bacteria | C | F(1)F(0) ATP synthase |
| | | | produces ATP from |
| | | | ADP in the presence of |
| | | | a proton or sodium gradient. |
| | | | F-type ATPases consist of |
| | | | two structural domains, |
| | | | F(1) containing the |
| | | | extramembraneous catalytic |
| | | | core and F(0) containing |
| | | | the membrane proton |
| | | | channel, linked together by |
| | | | a central stalk and a |
| | | | peripheral stalk. During |
| | | | catalysis, ATP synthesis |
| | | | in the catalytic domain of |
| | | | F(1) is coupled via a |
| | | | rotary mechanism of the |
| | | | central stalk subunits to |
| | | | proton translocation |
| Cluster 143 | Bacteria | L | PD-(D/E)XK nuclease |
| | | | superfamily |
| Cluster 14471 | | | |
| Cluster 1456 | Bacteria | | |
| Cluster 14959 | Bacteria | V | MatE |
| Cluster 1704 | Bacteria | C | Aldehyde dehydrogenase |
| | | | family |
| Cluster 2040 | Bacteria | S | Putative exopolysaccharide |
| | | | Exporter (EPS-E) |
| Cluster 250 | Bacteria | G | Glycosyl hydrolase family 85 |
| Cluster 2546 | Bacteria | G | Alpha-L-fucosidase |
| Cluster 2563 | Bacteria | T | Forkhead associated domain |
| Cluster 2765 | Bacteria | G | Bacterial extracellular |
| | | | solute-binding protein |
| Cluster 2801 | Bacteria | C | Na H antiporter family |
| | | | protein |
| Cluster 2839 | Bacteria | S | AAA domain |
| Cluster 3126 | Bacteria | G | Bacterial extracellular |
| | | | solute-binding protein |
| Cluster 320 | Bacteria | J | Ribonuclease E/G family |
| Cluster 3358 | Bacteria | M | Enolase C-terminal domain- |
| | | | like |
| Cluster 3512 | Bacteria | G | Major Facilitator Superfamily |
| Cluster 3753 | | | |
| Cluster 4705 | Bacteria | K | Periplasmic binding protein |
| | | | domain |
| Cluster 5109 | Bacteria | F | Phosphoribosylformyl- |
| | | | glycinamidine cyclo-ligase |
| Cluster 5197 | Bacteria | K | Bacterial regulatory proteins, |
| | | | lacI family |
| Cluster 5301 | Bacteria | | |
| Cluster 5401 | | | |
| Cluster 5664 | Bacteria | P | Binding-protein-dependent |
| | | | transport system inner |
| | | | membrane component |
| Cluster 5716 | Bacteria | EP | Binding-protein-dependent |
| | | | transport system inner |
| | | | membrane component |
| Cluster 5827 | Bacteria | U | Binding-protein-dependent |
| | | | transport system inner |
| | | | membrane component |
| Cluster 6058 | Bacteria | K | Bacterial regulatory |
| | | | proteins, tetR family |
| Cluster 6120 | Bacteria | | |
| Cluster 6142 | Bacteria | | |
| Cluster 6148 | | | |
| Cluster 6168 | Bacteria | EP | oligopeptide transport |
| | | | protein of the ABC |
| | | | superfamily, ATP- |
| | | | binding component |
| Cluster 6333 | Bacteria | U | Binding-protein- |
| | | | dependent transport |
| | | | system inner membrane |
| | | | component |
| Cluster 6443 | Bacteria | EM | Dihydrodipicolinate |
| | | | synthetase family |
| Cluster 6500 | Bacteria | | |
| Cluster 6584 | Bacteria | P | Binding-protein- |
| | | | dependent transport |
| | | | system inner membrane |
| | | | component |
| Cluster 7251 | Bacteria | | |
| Cluster 7435 | Bacteria | IQ | KR domain |
| Cluster 7556 | Bacteria | K | TipAS antibiotic- |
| | | | recognition domain |
| Cluster 7961 | Bacteria | E | Rard protein |
| Cluster 8187 | | | |
| Cluster 8251 | Bacteria | S | AAA ATPase domain |
| Cluster 8370 | Bacteria | C | Aldo/keto reductase family |
| Cluster 8376 | Bacteria | L | endonuclease III |
| Cluster 889 | Bacteria | GM | ADP-glyceromanno-heptose |
| | | | 6-epimerase activity |
| Cluster 9183 | Bacteria | L | Uracil DNA glycosylase |
| | | | superfamily |
| Cluster 9218 | Bacteria | T | Forkhead associated domain |
| Cluster 9634 | | | |
| Cluster 980 | Bacteria | G | Glycosyl hydrolase |
| | | | family 20, domain 2 |

TABLE 2
B. longum transitional clade - NCC specific genes

| | | | Presence | Presence in n |
|---|---|---|---|---|
| | | | in n NCC | genomes from |
| gene_cluster | genome | locus_tag | genomes | any clade |
| Cluster 10535 | NCC5001, NCC5000, | 1_1027, 1_983, 1_1011, 1_1056, 1_1038 | 5 | 8 |
| | NCC5002, NCC5004, | | | |
| | NCC5003 | | | |
| Cluster 10742 | NCC5003, NCC5001, | 1_1460, 1_628, 1_1449, 1_623, 1_1439 | 5 | 5 |
| | NCC5002, NCC5004, | | | |
| | NCC5000 | | | |
| Cluster 10912 | NCC5001, NCC5002, | 1_956, 1_1086, 1_1049, 1_981, 1_1113 | 5 | 5 |
| | NCC5000, NCC5004, | | | |
| | NCC5003 | | | |
| Cluster 12123 | NCC5004, NCC5003, | 1_1096, 1_998, 1_1067, 1_971, 1_942 | 5 | 5 |
| | NCC5001, NCC5002, | | | |
| | NCC5000 | | | |
| Cluster 12245 | NCC5002, NCC5001, | 1_1087, 1_955, 1_980, 1_1114, 1_1050 | 5 | 5 |
| | NCC5004, NCC5003, | | | |
| | NCC5000 | | | |
| Cluster 12381 | NCC5002, NCC5001, | 1_568, 1_1520, 1_650, 1_1438, 1_571 | 5 | 5 |
| | NCC5003, NCC5004, | | | |
| | NCC5000 | | | |
| Cluster 1274 | NCC5004, NCC5002, | 1_501, 1_1575, 1 1583, 1_532, 1_1561 | 5 | 5 |
| | NCC5003, NCC5001, | | | |
| | NCC5000 | | | |
| Cluster 1378 | NCC5001, NCC5000, | 1_535, 1_1558, 1_1572, 1_504, 1_1580 | 5 | 5 |
| | NCC5002, NCC5004, | | | |
| | NCC5003 | | | |
| Cluster 13922 | NCC5003, NCC5001, | 1_997, 1_1068, 1_941, 1_970, 1_1097 | 5 | 5 |
| | NCC5000, NCC5002, | | | |
| | NCC5004 | | | |
| Cluster 13960 | NCC5002, NCC5004, | 1_1055, 1_1012, 1_1018, 1_987, 1_1082 | 5 | 8 |
| | NCC5000, NCC5001, | | | |
| | NCC5003 | | | |
| Cluster 14054 | NCC5001, NCC5003, | 1_1835, 1_307, 1_1760, 1_277, 1_238 | 5 | 5 |
| | NCC5004, NCC5000, | | | |
| | NCC5002 | | | |
| Cluster 14471 | NCC5001, NCC5004, | 1_1066, 1_1095, 1_943, 1_999, 1_972 | 5 | 5 |
| | NCC5000, NCC5003, | | | |
| | NCC5002 | | | |
| Cluster 14959 | NCC5004, NCC5002, | 1_1126, 1_942, 1_967, 1_892, 1_1098 | 5 | 8 |
| | NCC5003, NCC5000, | | | |
| | NCC5001 | | | |
| Cluster 1704 | NCC5001, NCC5004, | 1_1519, 1_1437, 1_651, 1_572, 1_569 | 5 | 5 |
| | NCC5003, NCC5000, | | | |
| | NCC5002 | | | |
| Cluster 2040 | NCC5000, NCC5001, | 1_1559, 1_534, 1_503, 1_1581, 1_1573 | 5 | 5 |
| | NCC5004, NCC5003, | | | |
| | NCC5002 | | | |
| Cluster 2563 | NCC5000, NCC5003, | 1_1829, 1_1821, 1_235, 1_257, 1_1852 | 5 | 8 |
| | NCC5001, NCC5004, | | | |
| | NCC5002 | | | |
| Cluster 320 | NCC5004, NCC5003, | 1_226, 1_1850, 1_206, 1_1882, 1_1879 | 5 | 8 |
| | NCC5001, NCC5002, | | | |
| | NCC5000 | | | |
| Cluster 3358 | NCC5001, NCC5003, | 1_199, 1_1857, 1_1886, 1_219, 1_1889 | 5 | 8 |
| | NCC5000, NCC5004, | | | |
| | NCC5002 | | | |
| Cluster 3512 | NCC5001, NCC5003, | 1_201, 1_1855, 1_221, 1_1884, 1_1887 | 5 | 8 |
| | NCC5004, NCC5000, | | | |
| | NCC5002 | | | |
| Cluster 5109 | NCC5004, NCC5002, | 1_1435, 1_571, 1_653, 1_574, 1_1517 | 5 | 8 |
| | NCC5003, NCC5000, | | | |
| | NCC5001 | | | |
| Cluster 5301 | NCC5003, NCC5004, | 1_1000, 1_1094, 1_973, 1_1065, 1_944 | 5 | 5 |
| | NCC5002, NCC5001, | | | |
| | NCC5000 | | | |
| Cluster 5716 | NCC5004, NCC5001, | 1_1488, 1_1574, 1_518, 1_599, 1_521 | 5 | 8 |
| | NCC5002, NCC5003, | | | |
| | NCC5000 | | | |
| Cluster 6148 | NCC5004, NCC5000, | 1_2050, 1_14, 1_15, 1_2096, 1_14 | 5 | 7 |
| | NCC5003, NCC5001, | | | |
| | NCC5002 | | | |
| Cluster 6168 | NCC5003, NCC5002, | 1_600, 1_519, 1_1573, 1_522, 1_1487 | 5 | 8 |
| | NCC5001, NCC5000, | | | |
| | NCC5004 | | | |
| Cluster 6443 | NCC5003, NCC5001, | 1_1853, 1_203, 1_1882, 1_223, 1_1885 | 5 | 8 |
| | NCC5000, NCC5004, | | | |
| | NCC5002 | | | |
| Cluster 7435 | NCC5002, NCC5004, | 1_1888, 1_220, 1_200, 1_1856, 1_1885 | 5 | 8 |
| | NCC5001, NCC5003, | | | |
| | NCC5000 | | | |
| Cluster 7961 | NCC5004, NCC5002, | 1_1102, 1_968, 1_932, 1_1070, 1_992 | 5 | 5 |
| | NCC5000, NCC5001, | | | |
| | NCC5003 | | | |
| Cluster 8187 | NCC5001, NCC5002, | 1_2095, 1_15, 1_2049, 1_16, 1_15 | 5 | 8 |
| | NCC5004, NCC5003, | | | |
| | NCC5000 | | | |
| Cluster 8251 | NCC5002, NCC5001, | 1_21, 1_2087, 1_51, 1_2013, 1_49 | 5 | 5 |
| | NCC5003, NCC5004, | | | |
| | NCC5000 | | | |
| Cluster 8376 | NCC5000, NCC5004, | 1_619, 1_1389, 1_618, 1_1449, 1_699 | 5 | 8 |
| | NCC5002, NCC5001, | | | |
| | NCC5003 | | | |

| | Predicted | | | |
|---|---|---|---|---|
| | protein | | | |
| gene cluster | name | Gene Ontology terms | EC number | KEGG ko |
| Cluster 10535 | | | | |
| Cluster 10742 | | | | |
| Cluster 10912 | mepA_6 | | | |
| Cluster 12123 | | | | |
| Cluster 12245 | | | | |
| Cluster 12381 | | | 3.2.1.23 | ko: K01190 |
| Cluster 1274 | | | | |
| Cluster 1378 | cotH | | | ko: K06330 |
| Cluster 13922 | | | 2.1.1.72, 3.1.21.3, | ko: K01153, ko: |
| | | | 3.1.21.4 | K01155, ko: K03427, |
| | | | | ko: K09384 |
| Cluster 13960 | | | | |
| Cluster 14054 | atpE | GO: 0003674, GO: 0003824, GO: 0005215, GO: 0005575, | | ko: K02110 |
| | | GO: 0005622, GO: 0005623, GO: 0006139, GO: 0006163, | | |
| | | GO: 0006164, GO: 0006725, GO: 0006753, GO: 0006754, | | |
| | | GO: 0006793, GO: 0006796, GO: 0006807, GO: 0006810, | | |
| | | GO: 0006811, GO: 0006812, GO: 0008150, GO: 0008152, | | |
| | | GO: 0008324, GO: 0009058, GO: 0009117, GO: 0009123, | | |
| | | GO: 0009124, GO: 0009126, GO: 0009127, GO: 0009141, | | |
| | | GO: 0009142, GO: 0009144, GO: 0009145, GO: 0009150, | | |
| | | GO: 0009152, GO: 0009156, GO: 0009161, GO: 0009165, | | |
| | | GO: 0009167, GO: 0009168, GO: 0009199, GO: 0009201, | | |
| | | GO: 0009205, GO: 0009206, GO: 0009259, GO: 0009260, | | |
| | | GO: 0009987, GO: 0015075, GO: 0015077, GO: 0015078, | | |
| | | GO: 0015318, GO: 0015399, GO: 0015405, GO: 0015672, | | |
| | | GO: 0015985, GO: 0015986, GO: 0016020, GO: 0016462, | | |
| | | GO: 0016469, GO: 0016787, GO: 0016817, GO: 0016818, | | |
| | | GO: 0016887, GO: 0017111, GO: 0017144, GO: 0018130, | | |
| | | GO: 0019438, GO: 0019637, GO: 0019693, GO: 0019829, | | |
| | | GO: 0022804, GO: 0022853, GO: 0022857, GO: 0022890, | | |
| | | GO: 0032991, GO: 0033177, GO: 0034220, GO: 0034641, | | |
| | | GO: 0034654, GO: 0042623, GO: 0042625, GO: 0042626, | | |
| | | GO: 0043492, GO: 0044237, GO: 0044238, GO: 0044249, | | |
| | | GO: 0044271, GO: 0044281, GO: 0044424, GO: 0044425, | | |
| | | GO: 0044464, GO: 0044769, GO: 0045259, GO: 0045263, | | |
| | | GO: 0046034, GO: 0046390, GO: 0046483, GO: 0046933, | | |
| | | GO: 0051179, GO: 0051234, GO: 0055085, GO: 0055086, | | |
| | | GO: 0071704, GO: 0072521, GO: 0072522, GO: 0090407, | | |
| | | GO: 0090662, GO: 0098655, GO: 0098660, GO: 0098662, | | |
| | | GO: 0098796, GO: 0099131, GO: 0099132, GO: 1901135, | | |
| | | GO: 1901137, GO: 1901293, GO: 1901360, GO: 1901362, | | |
| | | GO: 1901564, GO: 1901566, GO: 1901576, GO: 1902600 | | |
| Cluster 14471 | | | | |
| Cluster 14959 | mepA | | | ko: K18908 |
| Cluster 1704 | aldH | | 1.2.1.3, 1.2.1.5 | ko: K00128, ko: |
| | | | | K00129 |
| Cluster 2040 | pelG | | | ko: K21012 |
| Cluster 2563 | | | | |
| Cluster 320 | rne | GO: 0006139, GO: 0006364, GO: 0006396, GO: 0006397, | 3.1.26.12 | ko: K08300, ko: |
| | | GO: 0006725, GO: 0006807, GO: 0008150, GO: 0008152, | | K08301 |
| | | GO: 0009987, GO: 0010467, GO: 0016070, GO: 0016071, | | |
| | | GO: 0016072, GO: 0022613, GO: 0034470, GO: 0034641, | | |
| | | GO: 0034660, GO: 0042254, GO: 0043170, GO: 0044085, | | |
| | | GO: 0044237, GO: 0044238, GO: 0046483, GO: 0071704, | | |
| | | GO: 0071840, GO: 0090304, GO: 1901360 | | |
| Cluster 3358 | | | 4.2.1.68 | ko: K18334 |
| Cluster 3512 | fucP | | | ko: K02429 |
| Cluster 5109 | purM | GO: 0003674, GO: 0003824, GO: 0004641, GO: 0005575, | 6.3.3.1, 6.3.4.13 | ko: K01933, ko: |
| | | GO: 0005622, GO: 0005623, GO: 0005737, GO: 0005829, | | K11788 |
| | | GO: 0006139, GO: 0006163, GO: 0006164, GO: 0006725, | | |
| | | GO: 0006753, GO: 0006793, GO: 0006796, GO: 0006807, | | |
| | | GO: 0008150, GO: 0008152, GO: 0009058, GO: 0009117, | | |
| | | GO: 0009150, GO: 0009152, GO: 0009165, GO: 0009259, | | |
| | | GO: 0009260, GO: 0009987, GO: 0016874, GO: 0016879, | | |
| | | GO: 0016882, GO: 0018130, GO: 0019438, GO: 0019637, | | |
| | | GO: 0019693, GO: 0034641, GO: 0034654, GO: 0044237, | | |
| | | GO: 0044238, GO: 0044249, GO: 0044271, GO: 0044281, | | |
| | | GO: 0044424, GO: 0044444, GO: 0044464, GO: 0046390, | | |
| | | GO: 0046483, GO: 0055086, GO: 0071704, GO: 0072521, | | |
| | | GO: 0072522, GO: 0090407, GO: 1901135, GO: 1901137, | | |
| | | GO: 1901293, GO: 1901360, GO: 1901362, GO: 1901564, | | |
| | | GO: 1901566, GO: 1901576 | | |
| Cluster 5301 | | | | |
| Cluster 5716 | | | | ko: K02034, ko: |
| | | | | K15582 |
| Cluster 6148 | | | | |
| Cluster 6168 | oppD | | | ko: K02031 |
| Cluster 6443 | dapA | | 4.1.2.28, 4.3.3.7 | ko: K01714, ko: |
| | | | | K22397 |
| Cluster 7435 | | | | |
| Cluster 7961 | | | 3.4.17.13 | ko: K01297 |
| Cluster 8187 | | | | |
| Cluster 8251 | | | | |
| Cluster 8376 | magIII | | | ko: K07457 |

| gene_cluster | KEGG_Pathway | KEGG_Module | KEGG_Reaction | KEGG_rclass |
|---|---|---|---|---|
| Cluster 10535 | | | | |
| Cluster 10742 | | | | |
| Cluster 10912 | | | | |
| Cluster 12123 | | | | |
| Cluster 12245 | | | | |
| Cluster 12381 | ko00052, ko00511, ko00600, | | R01105, R01678, R03355, | RC00049, RC00452 |
| | ko01100, map00052, map00511, | | R04783, R06114 | |
| | map00600, map01100 | | | |
| Cluster 1274 | | | | |
| Cluster 1378 | | | | |
| Cluster 13922 | | | | |
| Cluster 13960 | | | | |
| Cluster 14054 | ko00190, ko00195, ko01100, | M00157 | | |
| | map00190, map00195, | | | |
| | map01100 | | | |
| Cluster 14471 | | | | |
| Cluster 14959 | | M00705 | | |
| Cluster 1704 | ko00010, ko00053, ko00071, | M00135 | R00264, R00631, R00710, | RC00047, RC00071, |
| | ko00280, ko00310, ko00330, | | R00711, R00904, R01752, | RC00080, RC00186, |
| | ko00340, ko00350, k000360, | | R01986, R02536, R02537, | RC00218, RC00242, |
| | ko00380, ko00410, ko00561, | | R02549, R02678, R02695, | RC00816, RC01500, |
| | ko00620, ko00625, ko00903, | | R02697, R02940, R02957, | RC01735 |
| | ko00980, ko00981, ko00982, | | R03283, R03300, R03302, | |
| | ko01100, ko01110, ko01120, | | R03869, R04065, R04506, | |
| | ko01130, ko05204, map00010, | | R04882, R04883, R04888, | |
| | map00053, map00071, map00280, | | R04889, R04891, R04892, | |
| | map00310, map00330, map00340, | | R04903, R04996, R05050, | |
| | map00350, map00360, map00380, | | R05237, R05238, R05286, | |
| | map00410, map00561, map00620, | | R06366, R07104, R08146, | |
| | map00625, map00903, map00980, | | R08282, R08283, R08307 | |
| | map00981, map00982, map01100, | | | |
| | map01110, map01120, map01130, | | | |
| | map05204 | | | |
| Cluster 2040 | ko02025, map02025 | | | |
| Cluster 2563 | | | | |
| Cluster 320 | ko03018, map03018 | M00394 | | |
| Cluster 3358 | ko00051, ko01120, map00051, | | R03688 | RC00543 |
| | map01120 | | | |
| Cluster 3512 | | | | |
| Cluster 5109 | ko00230, ko01100, ko01110, | M00048 | R04144, R04208 | RC00090, RC00166, |
| | ko01130, map00230, map01100, | | | RC01100 |
| | map01110, map01130 | | | |
| Cluster 5301 | | | | |
| Cluster 5716 | ko01501, ko02010, ko02024, | M00239, M00439 | | |
| | map01501, map02010, map02024 | | | |
| Cluster 6148 | | | | |
| Cluster 6168 | ko02024, map02024 | M00239 | | |
| Cluster 6443 | ko00040, ko00261, ko00300, | M00016, M00525, M00526, M00527 | R01782, R10147 | RC00307, RC00572, |
| | ko01100, ko01110, ko01120, | | | RC03062, RC03063 |
| | ko01130, ko01230, map00040, | | | |
| | map00261, map00300, map01100, | | | |
| | map01110, map01120, map01130, | | | |
| | map01230 | | | |
| Cluster 7435 | | | | |
| Cluster 7961 | | | | |
| Cluster 8187 | | | | |
| Cluster 8251 | | | | |
| Cluster 8376 | | | | |

| gene cluster | BRITE | KEGG TC | CAZy | BiGG Reaction |
|---|---|---|---|---|
| Cluster 10535 | | | | |
| Cluster 10742 | | | | |
| Cluster 10912 | | | | |
| Cluster 12123 | | | | |
| Cluster 12245 | | | | |
| Cluster 12381 | ko00000, ko00001, ko01000 | | | |
| Cluster 1274 | | | | |
| Cluster 1378 | ko00000 | | | |
| Cluster 13922 | ko00000, ko01000, ko02048 | | | |
| Cluster 13960 | | | | |
| Cluster 14054 | ko00000, ko00001, ko00002, | 3.A.2.1 | | |
| | ko00194 | | | |
| Cluster 14471 | | | | |
| Cluster 14959 | ko00000, ko00002, ko01504, | 2.A.66.1.13 | | |
| | ko02000 | | | |
| Cluster 1704 | ko00000, ko00001, ko00002, | | | |
| | ko01000 | | | |
| Cluster 2040 | ko00000, ko00001 | | | |
| Cluster 2563 | | | | |
| Cluster 320 | ko00000, ko00001, ko00002, | | | |
| | ko01000, ko03009, ko03019 | | | |
| Cluster 3358 | ko00000, ko00001, ko01000 | | | |
| Cluster 3512 | ko00000, ko02000 | 2.A.1.7 | | |
| Cluster 5109 | ko00000, ko00001, ko00002, | | | iAF987.Gmet_1844, |
| | ko01000 | | | iECSF_1327.ECSF |
| | | | | 2340 |
| Cluster 5301 | | | | |
| Cluster 5716 | ko00000, ko00001, ko00002, | 3.A.1.5, 3.A.1.5.1, 3.A.1.5.18, | | |
| | ko02000 | 3.A.1.5.19, 3.A.1.5.25 | | |
| Cluster 6148 | | | | |
| Cluster 6168 | ko00000, ko00001, ko00002, | 3.A.1.5 | | |
| | ko02000 | | | |
| Cluster 6443 | ko00000, ko00001, ko00002, | | | |
| | ko01000 | | | |
| Cluster 7435 | | | | |
| Cluster 7961 | ko00000, ko01000, ko01002, | | | |
| | ko01011 | | | |
| Cluster 8187 | | | | |
| Cluster 8251 | | | | |
| Cluster 8376 | ko00000 | | | |

| gene_cluster | tax_scope | COG_Functional_Category | eggNOG_free_text_description |
|---|---|---|---|
| Cluster 10535 | Bacteria | | |
| Cluster 10742 | Bacteria | K | Bacterial regulatory |
| | | | proteins, tetR family |
| Cluster 10912 | Bacteria | V | MatE |
| Cluster 12123 | Bacteria | | |
| Cluster 12245 | | | |
| Cluster 12381 | Bacteria | G | Domain of unknown |
| | | | function (DUF4982) |
| Cluster 1274 | Bacteria | S | Uncharacterised |
| | | | protein conserved in |
| | | | bacteria (DUF2194) |
| Cluster 1378 | Bacteria | M | CotH kinase protein |
| Cluster 13922 | Bacteria | L | N-6 DNA methylase |
| Cluster 13960 | Bacteria | | |
| Cluster 14054 | Bacteria | C | F(1)F(0) ATP |
| | | | synthase produces |
| | | | ATP from ADP in the |
| | | | presence of a proton |
| | | | or sodium gradient. F- |
| | | | type ATPases consist |
| | | | of two structural |
| | | | domains, F(1) |
| | | | containing the |
| | | | extramembraneous |
| | | | catalytic core and F(0) |
| | | | containing the |
| | | | membrane proton |
| | | | channel, linked |
| | | | together by a central |
| | | | stalk and a peripheral |
| | | | stalk. During |
| | | | catalysis, ATP |
| | | | synthesis in the |
| | | | catalytic domain of |
| | | | F(1) is coupled via a |
| | | | rotary mechanism of |
| | | | the central stalk |
| | | | subunits to proton |
| | | | translocation |
| Cluster 14471 | | | |
| Cluster 14959 | Bacteria | V | MatE |
| Cluster 1704 | Bacteria | C | Aldehyde |
| | | | dehydrogenase family |
| Cluster 2040 | Bacteria | S | Putative |
| | | | exopolysaccharide |
| | | | Exporter (EPS-E) |
| Cluster 2563 | Bacteria | T | Forkhead associated |
| | | | domain |
| Cluster 320 | Bacteria | J | Ribonuclease E/G |
| | | | family |
| Cluster 3358 | Bacteria | M | Enolase C-terminal |
| | | | domain-like |
| Cluster 3512 | Bacteria | G | Major Facilitator |
| | | | Superfamily |
| Cluster 5109 | Bacteria | F | Phosphoribosylformyl |
| | | | glycinamidine cyclo- |
| | | | ligase |
| Cluster 5301 | Bacteria | | |
| Cluster 5716 | Bacteria | EP | Binding-protein- |
| | | | dependent transport |
| | | | system inner |
| | | | membrane component |
| Cluster 6148 | | | |
| Cluster 6168 | Bacteria | EP | oligopeptide transport |
| | | | protein of the ABC |
| | | | superfamily, ATP- |
| | | | binding component |
| Cluster 6443 | Bacteria | EM | Dihydrodipicolinate |
| | | | synthetase family |
| Cluster 7435 | Bacteria | IQ | KR domain |
| Cluster 7961 | Bacteria | E | Rard protein |
| Cluster 8187 | | | |
| Cluster 8251 | Bacteria | S | AAA ATPase domain |
| Cluster 8376 | Bacteria | L | endonuclease III |

### Example 6—B. longum Transitional Clade MAGs

SEQ ID NOs 1-4010 set forth B. longum transitional clade MAGs from stool samples from various subject at various times from the Dhaka cohort and used in the Examples herein. Table 3 below summarizes the sequences provided in SEQ ID NOs. 1-4010.

Multiple MAGs were obtained for each MAG ID. The corresponding sequences of the multiple MAGs for each MAG TD is provided in Table 3. Further, each unique MAG is identified in the sequence listing, which is incorporated by reference, by a unique and arbitrary identifier, “MAG No.” This information is provided in the description of each sequence within the sequence listing. For example, SEQ TD NO: 1 has the description of “MAG 02-MAG No. k141_188”.

TABLE 3
Sequence Listing Information

| MAG ID or | SEQ ID | Species | |
|---|---|---|---|
| Deposit ID | NOs | Description | Subspecies or clade |
| R30_18m | 1-55 | B. Longum | Transitional clade |
| R206_24m | 56-165 | B. Longum | Transitional clade |
| R101_18m | 166-311 | B. Longum | Transitional clade |
| R42_10m | 312-393 | B. Longum | Transitional clade |
| R14_15m | 394-587 | B. Longum | Transitional clade |
| R14_18m | 588-746 | B. Longum | Transitional clade |
| R48_24m | 747-897 | B. Longum | Transitional clade |
| R117_24m | 898-1037 | B. Longum | Transitional clade |
| R13_10m | 1038-1106 | B. Longum | Transitional clade |
| R81_24m | 1107-1259 | B. Longum | Transitional clade |
| R157_18m | 1260-1374 | B. Longum | Transitional clade |
| R257_15m | 1375-1535 | B. Longum | Transitional clade |
| R221_24m | 1536-1682 | B. Longum | Transitional clade |
| R125_18m | 1683-1908 | B. Longum | Transitional clade |
| R17_18m | 1909-2082 | B. Longum | Transitional clade |
| R51_24m | 2083-2257 | B. Longum | Transitional clade |
| R119_18m | 2258-2345 | B. Longum | Transitional clade |
| R50_10m | 2346-2576 | B. Longum | Transitional clade |
| R222_15m | 2577-2627 | B. Longum | Transitional clade |
| R36_18m | 2628-2904 | B. Longum | Transitional clade |
| R19_24m | 2905-3089 | B. Longum | Transitional clade |
| R215_18m | 3090-3147 | B. Longum | Transitional clade |
| R53_2m | 3148-3282 | B. Longum | Transitional clade |
| R151_18m | 3283-3459 | B. Longum | Transitional clade |
| R85_18m | 3460-3677 | B. Longum | Transitional clade |
| R150_10m | 3678-3752 | B. Longum | Transitional clade |
| R35_2m | 3753-3865 | B. Longum | Transitional clade |
| R22_24m | 3866-4010 | B. Longum | Transitional clade |

### Example 7—B. longum Transitional Cade Strains

Newly described B. longum strains were isolated from the feces of breast-fed infants using Eugon Tomato Agar (ETA). Obtained isolates were sequenced using PacBio to obtain a fully closed assembled genome for each of the strain. Each strain was deposited in the internal Nestle Culture Collection (NCC, Lausanne, Switzerland) and at the Collection Nationale de Microorganisms (CNCM) at the Pasteur Institute (Paris, France) on May 11, 2021 together with their genome sequence data. The genome of the strains was compared by Average Nucleotide Identity (ANI) using OrthoAni (https://www.ezbiocloud.net/tools/orthoani) to other publicly available genomes representing the overall diversity of the B. longum species (Table 4), and to the Metagenomic Assembled Genomes (MAG) obtained from metagenomic sequences issued from infant feces of the same cohort.

The analysis demonstrates that the newly described strains group together with the MAGs obtained from the same cohort, defining a well delineated clade belonging to the B. longum species. Two previously isolated strains BSM11-5 and 3_mod are found to be grouped within this newly described clade. The clade is genetically different from B. longum subspecies longum (96.40% ANI) subspecies. The clade is related, while still clearly distinct, to B. longum subspecies. suis suillum (98.207%), and to the group of strains (JDM301, CMCC_P0001 and BXY01) previously suggested to be a new B. longum subspecies (O'Callaghan et al. 2015), sharing an identity of 98.260% to this group of strains.

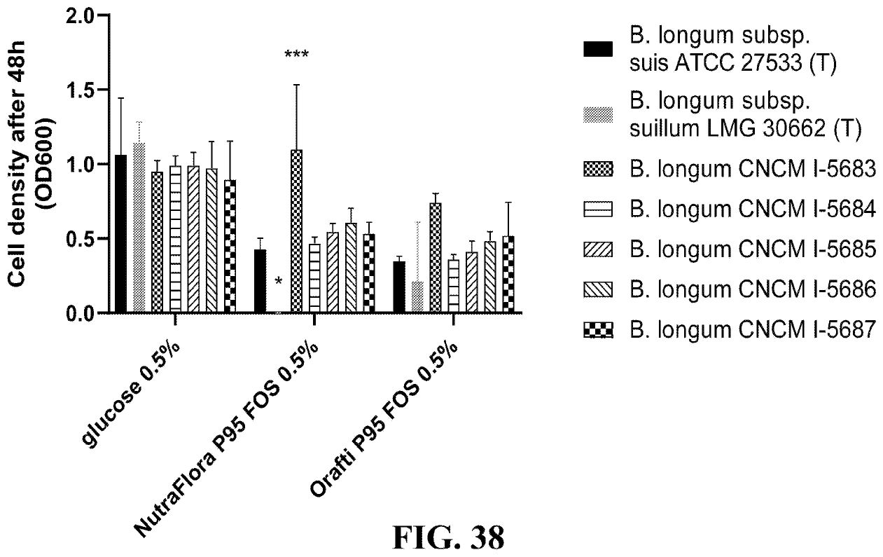

FIG. 38 shows Average Nucleotide Identity (ANI) UPGMA based phylogenetic tree. The scale represents the percentage (%) of identity at each branch point.

TABLE 4
List of genomes used for Average Nucleotide Identity (ANI) analysis
and their publicly available references. (T) stands for typestrain.

| | Strain number | Nestlé Culture | |
|---|---|---|---|
| | (CNCM | Collection | |
| | Accession/Registration | internal | Genome |
| Taxonomy | Number) | number | reference |
| B. longum | CNCM I-5683 | NCC 5000 | Available at the |
| | | | CNCM |
| B. longum | CNCM I-5684 | NCC 5001 | Available at the |
| | | | CNCM |
| B. longum | CNCM I-5685 | NCC 5002 | Available at the |
| | | | CNCM |
| B. longum | CNCM I-5686 | NCC 5003 | Available at the |
| | | | CNCM |
| B. longum | CNCM I-5687 | NCC 5004 | Available at the |
| | | | CNCM |
| B. longum subsp. suis | BSM11-5 | NA | GCF_001870705.1 |
| B. longum subsp. infantis | 3_mod | NA | GCF_902167615.1 |
| B. longum subsp. longum | JDM301 | NA | CP002010 |
| B. longum | BXY01 | NA | GCF_000730205.1 |
| B. longum subsp. longum | CMCC_P0001 | NA | GCF_000410595.1 |
| B. longum subsp suillum | SU-851 | NA | GCF_016882605.1 |
| B. longum subsp suillum | JCM19995 (T) | NCC 3079 | GCF_017132755 |
| B. longum subsp. suis | 2074B | NA | GCF_016759645.1 |
| B. longum subsp. suis | Su859 (T) | NA | GCF_900103055.1 |
| B. longum subsp. suis | DSM-20211 (T) | NA | GCF_000771285.1 |
| B. longum subsp. suis | LMG_21814 (T) | NA | GCF_000741625.1 |
| B. longum subsp. suis | 209B | NA | GCF_016759725.1 |
| B. longum subsp. suis | UMA026 | NA | PHUM01000001 |
| B. longum subsp. suis | AGR2137 | NA | GCF_000421385.1 |
| B. longum subsp. longum | NCC 2075 | NCC 2705 | GCF_000007525.1 |
| B. longum subsp. longum | DJO10A | NA | GCF_000008945.1 |
| B. longum subsp. longum | 157F | NA | GCF_000196575.1 |
| B. longum subsp. longum | DSM_20219 (T) | NA | GCF_900104835.1 |
| B. longum subsp. longum | JCM_1217 | NA | GCF_000196555.1 |
| B. longum subsp infantis | ATCC_15697 (T) | NA | JDTT01000001 |
| B. longum subsp infantis | Bi-26 | NA | GCF_004919065.2 |
| B. longum subsp infantis | JCM_11347 | NA | CP062951 |

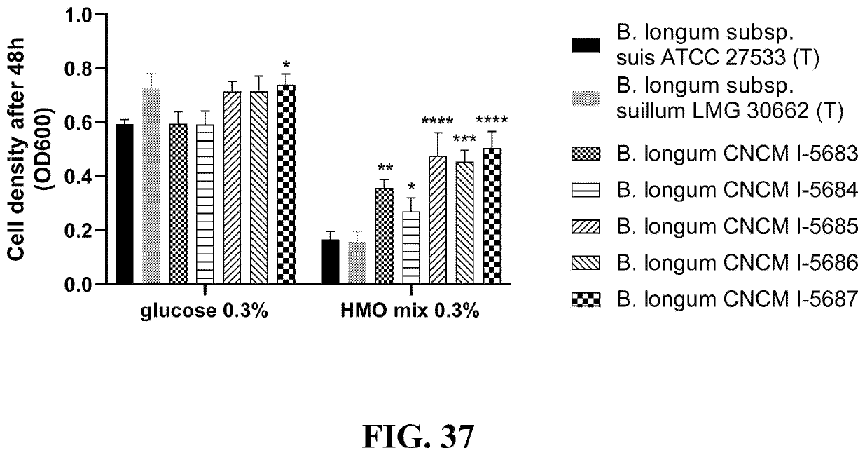

All strains retrieved from the Nestle Culture Collection (Table 5) were reactivated from a freeze-dried stock, using two successive culturing steps (16 h, 37° C., anaerobiosis) in MRS supplemented with 0.05% cysteine (MRSc). Reactivated cultures were then centrifuged, washed, and resuspended in 1 volume of PBS. Washed cells were used to inoculate MRS based medium without a carbon source (MRSc-C) (10 g 1-1 of bacto proteose peptone n° 3, 5 g 1-1 bacto yeast extract, 1 g 1-1 Tween 80, 2 g 1-1 di-ammonium hydrogen citrate, 5 g 1-1 sodium acetate, 0.1 g 1-1 magnesium sulphate, 0.05 g 1-1 manganese sulfate, 2 g 1-1 di-sodium phosphate, 0.5 g 1-1 cysteine) in which different carbohydrates were added (0.3% for FIG. 37 and 0.5% for FIG. 38). Growth was then performed in a 96 well microplate, with a volume of 200 μl per well. Incubation was performed in anaerobiosis for 48 h, and optical density was measured in a spectrophotometer at 600 nm.

TABLE 5
List of the strains (and corresponding
numbers) used for the growth studies

| | Internationally | Nestlé Culture |
|---|---|---|
| | recognized | Collection |
| Taxonomy | deposit number | internal number |
| Bifidobacterium longum | ATCC 27533 (T) | NCC 295 |
| subsp. suis | | |
| Bifidobacterium longum | LMG 30662 (T) | NCC 3079 |
| subsp. suillum | | |
| Bifidobacterium longum | CNCM I-5683 | NCC 5000 |
| Bifidobacterium longum | CNCM I-5684 | NCC 5001 |
| Bifidobacterium longum | CNCM I-5685 | NCC 5002 |
| Bifidobacterium longum | CNCM I-5686 | NCC 5003 |
| Bifidobacterium longum | CNCM I-5687 | NCC 5004 |

Genome sequences for B. longum transitional strains NCC 5000 (CNCM I-5683), NCC 5001 (CNCM I-5684), NCC 5002 (CNCM I-5685), NCC 5003 (CNCM I-5686) and NCC 5004 (CNCM I-5687) are also available via Joint Genome Project (JGI) Study number: Gs0156595 (https://genome.jgi.doe.gov/portal/). Analysis project numbers for each genome are as in Table 6.

TABLE 6
JGI Study ID Nos. for B. longum strains

| | |
|---|---|
| B. longum NCC 5000 (CNCM I-5683) | Ga0527908 |
| B. longum NCC 5001 (CNCM I-5684) | Ga0529016 |
| B. longum NCC 5002 (CNCM I-5685) | Ga0529017 |
| B. longum NCC 5003 (CNCM I-5686) | Ga0529018 |
| B. longum NCC 5004 (CNCM I-5687) | Ga0529019 |

In FIG. 37, strains were grown in a mixture of HMO representing a median of the concentration found in human breast milk between 3 weeks and 6 months after birth (Table 7) (Lefebvre et al 2020; Austin et al. 2016). As control, strains were grown in MRSc-C without any sugar addition (blank) and in 0.3% of glucose. While all tested strains produced a cell density close to 1 in glucose, it was different regarding growth on human milk derived carbohydrates. Results show that the newly described B. longum strains could all grew on the carbohydrates derived from human milk, while strain belonging to the closest genetic relative subspecies (B. longum subspecies suis & B. longum subspecies suillum) did not.

TABLE 7
HMO mixture representing the median concentrations found
in human breast milk between 3 weeks and 6 months after
birth (Lefebvre et al 2020; Austin et al. 2016)

| Human Milk | | |
|---|---|---|
| Oligosaccharides | Abbreviation | Concentration mg/L |
| 2′-O-Fucosyllactose | 2′FL | 1624 |
| Difucosyllactose | DFL | 235 |
| Lacto-N-Tetraose | LNT | 508 |
| 6′-Sialyllactose | 6′SL | 160 |
| 3′-Sialyllactose | 3′SL | 113 |
| 3-Fucosyllactose | 3-FL | 782 |
| Lacto-N-neotetraose | LNnT | 93 |

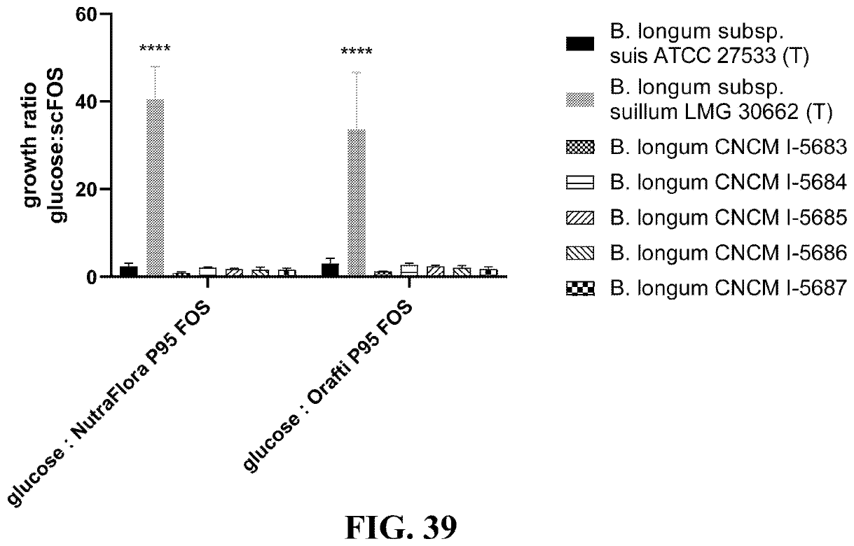

In FIG. 38 strains were grown in commercially available fructo-oligosaccharide (FOS) fibers (NutraFlora FOS [Ingredion Korea Inc, Gyunggi-do, Korea]; Orafti P95 [BENEO GmbH, Mannheim, Germany]), at a final concentration of 0.5%. As control, strains were grown in MRSc-C without any sugar addition and in 0.5% of glucose. All tested strains grew in glucose, producing a final optical density of 1 and above. All the newly described B. longum strains could grow well on the two commercial FOS. Out of the two strains belonging to the closest genetic relative subspecies, B. longum subspecies suis ATCC 27533 (T) grew relatively well on both FOS substrates, while B. longum subspecies suillum LMG 30662 (T) did not, especially on the NutraFlora fiber (FIG. 38). It is interesting to observe that all newly described B. longum strains were growing on FOS at a ratio close to that of glucose, while the B. longum subspecies suillum LMG 30662 (T) did show a preference for glucose (FIG. 39).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A synthetic formulation comprising:
one or more Bifidobacterium longum (B. longum) transitional clade microorganisms which has at least 98.6% Average Nucleotide Identity (ANI) with at least one strain selected in the group consisting of: CNCM I-5683, CNCM I-5684, CNCM I-5685, CNCM I-5686 and CNCM I-5687.

2. A synthetic formulation of claim 1 comprising one or more Bifidobacterium longum (B. longum) transitional clade microorganisms characterized by the presence of all genes from Table 1.

3. A synthetic formulation comprising one or more Bifidobacterium longum (B. longum) transitional clade microorganisms characterized by the presence of all genes from Table 2.

4. The synthetic formulation of any one of claims 1-3, wherein the one or more B. longum transitional clade microorganisms is not of the subspecies B. longum subspecies longum or B. longum subspecies infantis.

5. The synthetic formulation of any one of claims 1-4, wherein the one or more B. longum transitional clade microorganisms are isolated from a human.

6. The synthetic formulation of any one of claims 1-5, further comprising a fat source, a protein source, a carbohydrate source, a dietary fiber source, or a combination thereof.

7. The synthetic formulation of claim 7, wherein the fat source is a milk derived fat source or equivalent thereof, wherein the protein source is a milk derived protein source or equivalent thereof, and/or wherein the carbohydrate source is a milk derived carbohydrate source or equivalent thereof.

8. The synthetic formulation of any one of claims 6-7, wherein the dietary fiber is a prebiotic fiber.

9. The synthetic formulation of claim 8, wherein the one or more B. longum microorganisms are associated with the prebiotic fiber.

10. The synthetic formulation of any one of claims 6-9, wherein the synthetic formulation is a milk fortifier or milk replacement.

11. The synthetic formulation of any one of claims 6-10, wherein the synthetic formulation is adapted for infant and/or young child use.

12. A genetically modified microorganism or population thereof, wherein the genetically modified microorganism is not of a Bifidobacterium longum transitional clade and is genetically modified to comprise modified expression of one or more genes from Table 1.

13. A synthetic formulation comprising the genetically modified microorganism or population thereof of claim 12.

14. A method for, in an infant, promoting/assisting transition from a milk-based diet to solid food and/or promoting gut microbiota adapted to metabolize both milk derived carbohydrates and fibers or any derivative thereof comprising:
administering a synthetic formulation of any one of claims 1-11 or 13 to the infant and/or young child.

15. A method for promoting/assisting transition from a milk-based diet to solid food and/or promoting gut microbiota adapted to metabolize both milk derived carbohydrates and fibers or a derivative thereof in an infant and/or young child comprising:
administering, to the infant and/or young child, one or more Bifidobacterium longum (B. longum) transitional clade microorganism which has at least 98.6 Average Nucleotide Identify (ANI) with at least one strain selected in the group consisting of: CNCM I-5683, CNCM I-5684, CNCM I-5685, CNCM I-5686 and CNCM I-5687.

16. The method of claim 15, wherein the one or more Bifidobacterium longum (B. longum) microorganisms characterized by the presence of all genes from Table 1.

17. The method of any one of claims 15-16, wherein the one or more B. longum microorganisms is not of the subspecies B. longum subsp. longum or B. longum subsp. Infantis.

18. The method of any one of claims 15-17, wherein the one or more B. longum microorganisms are isolated from a human.

19. A method of identifying a gastrointestinal microbiome modulating agent comprising:
administering an amount of a test agent to a subject; and
determining a microbiome structure of a gastrointestinal tract of the subject by at least determining the relative abundance of one or more one or more Bifidobacterium longum (B. longum) microorganisms characterized by at least 98.6% Average Nucleotide identity (ANI) with at least one strain selected in the group consisting of: CNCM I-5683, CNCM I-5684, CNCM I-5685, CNCM I-5686 and CNCM I-5687,
wherein the gastrointestinal microbiome modulating agent is effective to increase or decrease the relative abundance of one or more of the one or more B. longum microorganism characterized by the presence of the B. longum transitional clade genes.

20. The method of claim 19, wherein the one or more B. longum microorganisms is characterized by the presence of all genes from Table 1.

21. The method of any one of claims 19-20, wherein the one or more B. longum microorganisms is not of the subspecies B. longum subspecies longum or B. longum subspecies infantis.

22. The method of any one of claims 19-21, wherein the one or more B. longum microorganisms are isolated from a human.

23. The method of any one of claims 19-22, wherein the test agent is provided in a formulation.

24. A Bifidobacterium longum (B. longum) microorganism characterized by at least 98.6% Average Nucleotide identity (ANI) with at least one strain selected in the group consisting of: CNCM I-5683, CNCM I-5684, CNCM I-5685, CNCM I-5686 and CNCM I-5687, and combinations thereof.

25. A Bifidobacterium longum (B. longum) microorganism as in claim 25, wherein the B. longum microorganism is characterized by the presence of all genes from Table 1.

26. A Bifidobacterium longum (B. longum) microorganism of claims 24-25, wherein the B. longum microorganism is not of the subspecies B. longum subspecies longum or B. longum subspecies infantis.

27. A Bifidobacterium longum (B. longum) microorganism of any one of claims 24-26, which is isolated from human.

28. A combination of one or more microorganisms as described in any one of claims 24-27, milk derived carbohydrates, and dietary fibers.

29. A synthetic composition comprising a microorganism as described in any one of claims 24-27 or a combination according to claim 28.

30. A synthetic composition according to claim 29 for use in promoting/assisting transition from a milk-based diet to solid food in infants (as of the age of 4 months) and/or in a young child and/or promoting in infants (as of the age of 4 months) and/or in a young child a gut microbiota adapted to metabolize both milk derived carbohydrates and fibers or any derivative thereof.

31. Use of a microorganism as described in any one of claims 24-28 or a combination of claim 29 for promoting/assisting transition from a milk-based diet to solid food in infants (as of the age of 4 months) and/or in a young child and/or in promoting in infants (as of the age of 4 months) and/or in a young child a gut microbiota adapted to metabolize both milk derived carbohydrates and fibers or any derivative thereof.

32. A method for promoting/assisting transition from a milk based diet to solid food in infants (as of the age of 4 months) and/or in a young child and/or in promoting in infants (as of the age of 4 months) and/or in a young child a gut microbiota adapted to metabolize both milk derived carbohydrates and fibers or any derivative thereof by administering a microorganism as described in any one of claims 24-28 or a combination of claim 29 to such infant and/or young child.
